(19) United States
(12) Patent Application Publication
(10) Pub. No.: US 2023/0380903 A1
(43) Pub. Date: Nov. 30, 2023

(54) Systems and Methods for Treating Cardiovascular Tissue

(71) Applicant: Amplitude Vascular Systems, Inc., Boston, MA (US)

(72) Inventors: Robert Chisena, Boston, MA (US); Hitinder Gurm, Boston, MA (US); Louis Cannon, Boston, MA (US)

(21) Appl. No.: 18/201,965

(22) Filed: May 25, 2023

Related U.S. Application Data
(60) Provisional application No. 63/346,703, filed on May 27, 2022.

(51) Int. Cl. A61B 18/26; A61B 17/22; A61M 25/10

(52) U.S. Cl. CPC A61B 18/26; A61B 17/22022; A61M 25/1002; A61B 2017/22081; A61B 2018/00369

(57) ABSTRACT

Systems for imparting pulsatile energy to cardiovascular tissue are provided. Aspects of the systems include a console assembly comprising a potential source, a manifold assembly operably connected to an output of the console assembly, wherein the manifold assembly comprises an oscillator configured to generate pulse energy from energy transmitted from the potential source and a catheter assembly operably connected to an output of the manifold assembly. Catheter assemblies of the present invention include a connector operably connecting the catheter assembly to the manifold assembly and configured to transduce a first pulse energy generated by the manifold assembly to a second pulse energy, a catheter comprising a fluidic passage operably connected to the output of the connector and configured to transmit the second pulse energy and a heart-tissue-conforming element configured to receive the second pulse energy transmitted through the fluidic passage of the catheter to apply pulsatile energy to cardiovascular tissue. Also provided are methods for imparting pulsatile energy to cardiovascular tissue, e.g., deploying a system so that a heart-tissue-conforming element of the system is adjacent to cardiovascular tissue and engaging the system in a manner that the heart-tissue-conforming element imparts energy to the cardiovascular tissue. In addition, standalone catheter assemblies as well as kits comprising components of the systems described herein are provided. The systems, assemblies, methods and kits find use in a variety of different applications, including balloon angioplasty applications or other catheter-based therapies or treatments.

### CROSS-REFERENCE TO RELATED APPLICATION

Pursuant to 35 U.S.C. § 119(e), this application claims priority to the filing date of U.S. provisional patent application Ser. No. 63/346,703 filed May 27, 2022, the disclosures of which application is incorporated herein by reference in its entirety.

### INTRODUCTION

Cardiovascular tissue, including heart valve tissue, is susceptible to atherosclerotic plaque build-up through a mechanism called atherosclerosis, which is the accumulation of fatty and calcified materials that cause stenosis, the narrowing of an arterial lumen, or the failure of heart valves to function properly. Calcific aortic valve stenosis is a frequent cause of aortic valve disease, which leads to syncope (fainting), dyspnea (shortness of breath), heart failure, and, ultimately, death. The prevalence of aortic stenosis increases sharply with age. It has been cited that up to one third of elderly patients have some form of evidence of calcific aortic valve stenosis with 25% prevalence in people over the age of 65 and almost 50% in those over the age of 85. Under normal circumstances, the aortic valve is composed of three leaflets, each of which is a thin (e.g., less than 1 mm), smooth, flexible, and mobile structure. In aortic stenosis, these leaflets become thickened, fibrosed, and calcified, resulting in reduced leaflet mobility and progressive valvular obstruction. Calcium deposition leads to increased stress in the leaflets, which leads to more injury of the vessel, which thereby leads to increased deposition of calcium.

Existing treatment options include catheter-based valvuloplasty. Typical catheter balloon shapes, however, tend to obstruct the flow of blood through the heart while inflated, limiting, for example, treatment time. In addition, overinflation of balloon aortic valvuloplasty can lead to aortic annulus rupture. Aortic valve area in diseased patients can be between 0.1 and 1.5 cm 2 and balloon aortic valvuloplasty delivers an averaging effect of the balloon pressure over such tissue area. That is, it does not deliver a focal pressure to, for example, the commissures of the valve but, instead, relies on squeezing of the valve leaflets between the annulus and the balloon.

Balloon aortic valvuloplasty is currently known as a bridge to transcatheter aortic valve replacement (TAVR) or surgical aortic valve replacement (SAVR) or as a means of delivering palliative care for those who are not indicated for TAVR or SAVR or as part of TAVR to facilitate delivery of the transcatheter valve. Isolated balloon aortic valvuloplasty is associated with a high rate of morbidity and has a high rate of restenosis. To the inventors' knowledge, existing perfusion catheters for use in such procedures lack stability as well as the radial force required to modify calcific valves and to separate valve commissures. In addition, to the inventors' knowledge, current treatments do not offer solutions for balloon aortic valvuloplasty providing localized or focal stress points to heart valve features, such as the commissures or valve leaflets, while also enabling perfusion and moreover do not offer mechanisms for locating features of cardiovascular tissue, such as the valve commissures, during treatment.

### SUMMARY

Therefore, there remains a need for improved systems and methods for successfully imparting pulsatile energy to cardiovascular tissue, e.g., heart valves, e.g., aortic valves, including, while providing perfusion past the cardiovascular tissue. For example, a more consistent balloon aortic valvuloplasty procedure that requires minimal rapid pacing and longer treatment periods could improve the preparation for transcatheter aortic valve replacement (TAVR) or for potentially longer lasting treatment of calcific aortic stenosis. Improved systems and methods for successfully imparting pulsatile energy to cardiovascular tissue could also result in more durable valvuloplasty and negate, or significantly delay, the need for TAVR or SAVR.

As described herein, the invention relates to systems and methods for imparting pulsatile energy to cardiovascular tissue, including in the context of balloon valvuloplasty treatments. Specifically, systems and methods of the present invention facilitate providing localized or focal stress points to cardiovascular tissue, such as aspects of cardiovascular tissue, e.g., a heart valve, e.g., the commissures or valve leaflets, while also providing perfusion to distal vessels such that the focal forces on, e.g., the commissures and leaflets are maintained while allowing blood to continue to perfuse to distal tissues. Such embodiments allow longer treatment durations facilitating the occurrence of calcium fatigue fracture in cardiovascular tissue, e.g., the valve commissures and leaflets.

Embodiments of systems of the present invention provide mechanisms for locating the valve commissures during treatment by, for example, providing outer balloons configured to vibrate prior to full valvuloplasty balloon expansion such that the balloons locate the path of least resistance (i.e., the valve commissures). The location and appropriate alignment of a pulsatile apparatus (i.e., a heart-tissue-conforming element) of an embodiment of a system of the invention may be confirmed using, for example, radiopaque markings on such pulsatile apparatus (e.g., on a balloon) or other identifying markers which, in some cases, may also be capable of verifying commissural alignment. Once located appropriately with respect to the cardiovascular tissue, further pulsation can be generated within the valve commissures to effect calcium fracture and separation of tissue (e.g., heart valve leaflets).

Systems for imparting pulsatile energy to cardiovascular tissue are provided. Aspects of the systems include a console assembly comprising a potential source, a manifold assembly operably connected to an output of the console assembly, wherein the manifold assembly comprises an oscillator configured to generate pulse energy from energy transmitted from the potential source and a catheter assembly operably connected to an output of the manifold assembly. Catheter assemblies of the present invention include a connector operably connecting the catheter assembly to the manifold assembly and configured to transduce a first pulse energy generated by the manifold assembly to a second pulse energy, a catheter comprising a fluidic passage operably connected to the output of the connector and configured to transmit the second pulse energy and a heart-tissue-conforming element configured to receive the second pulse energy transmitted through the fluidic passage of the catheter to apply pulsatile energy to cardiovascular tissue. Also provided are methods for imparting pulsatile energy to cardiovascular tissue, e.g., deploying a system so that a heart-tissue-conforming element of the system is adjacent to cardiovascular tissue and engaging the system in a manner that the heart-tissue-conforming element imparts energy to the cardiovascular tissue. In addition, standalone catheter assemblies as well as kits comprising components of the systems described herein are provided. The systems, assemblies, methods and kits find use in a variety of different applications, including balloon angioplasty applications or other catheter-based therapies or treatments.

### BRIEF DESCRIPTION OF THE FIGURES

FIG. 1 provides a schematic diagram of an embodiment of a system according to the present invention for imparting pulsatile energy to cardiovascular tissue.

FIGS. 2A and 2B provide schematic views of a console assembly of a system according to an embodiment of the invention.

FIGS. 3A and 3B depict views of an exemplary embodiment of a console assembly of a system according to an embodiment of the invention.

FIGS. 4A and 4B provide schematic views of a manifold assembly of a system according to an embodiment of the invention.

FIG. 5 provides a perspective view of manifold assembly of a system according to an embodiment of the invention.

FIGS. 6A to 6E depict multiple views of an embodiment of a connector of a system configured to deliver a low volume, high-frequency, and high-pressure pulse in accordance with embodiments of the invention.

FIG. 7 provides a schematic of an alternative connector configured to deliver a high-volume, low-frequency, and low-pressure pulse in accordance with embodiments of the invention.

FIGS. 8A to 8C depict multiple views of a heart-tissue-conforming element of a system according to an embodiment of the invention.

FIG. 9 depicts an embodiment of a heart-tissue-conforming element of a system according to an embodiment of the invention.

FIG. 10 depicts balloon pressurization measurements and corresponding electrocardiogram (ECG) readings.

FIGS. 11A to 11D depict multiple views of an embodiment of a heart-tissue-conforming element of a system according to an embodiment of the invention.

FIGS. 12A and 12B depict cross sectional views of heart valves.

FIGS. 13A to 13C depict cross section views of certain embodiments of heart-tissue-conforming elements for use with bicuspid valves, such as mitral valves.

FIGS. 14A and 14B depict an embodiment of a catheter assembly configured to enable perfusion past the heart-tissue-conforming element while the catheter assembly is engaged with cardiovascular tissue.

FIG. 15 depicts a schematic of control loop for controlling a system according to an embodiment of the invention.

FIG. 16 depicts a schematic of a robotic method for delivering therapy energy to a diseased valve from a control room.

FIG. 17 depicts pressure-volume curves of cardiovascular tissue before and after treatment with a system according to an embodiment of the invention.

FIGS. 18A and 18B provide an example of measurements of changes in tissue compliance obtained during treatment using a system according to the present invention.

FIG. 19 shows a graphical user interface (GUI) that an operator of a system of the present invention may interface with during a treatment procedure comprising imparting pulsatile energy to a heart valve.

FIG. 20 provides a photograph of an aspect of a catheter assembly according to an embodiment of the invention.

FIG. 21 provides various photographic views of the connector of the catheter assembly of FIG. 20.

FIG. 22 provides two examples of the experimentally measured pressure in a single balloon attached to the catheter assembly of FIG. 20 and force output from the single balloon.

### DETAILED DESCRIPTION

Systems for imparting pulsatile energy to cardiovascular tissue are provided. Aspects of the systems include a console assembly comprising a potential source, a manifold assembly operably connected to an output of the console assembly, wherein the manifold assembly comprises an oscillator configured to generate pulse energy from energy transmitted from the potential source and a catheter assembly operably connected to an output of the manifold assembly. Catheter assemblies of the present invention include a connector operably connecting the catheter assembly to the manifold assembly and configured to transduce a first pulse energy generated by the manifold assembly to a second pulse energy, a catheter comprising a fluidic passage operably connected to the output of the connector and configured to transmit the second pulse energy and a heart-tissue-conforming element configured to receive the second pulse energy transmitted through the fluidic passage of the catheter to apply pulsatile energy to cardiovascular tissue. Also provided are methods for imparting pulsatile energy to cardiovascular tissue, e.g., deploying a system so that a heart-tissue-conforming element of the system is adjacent to cardiovascular tissue and engaging the system in a manner that the heart-tissue-conforming element imparts energy to the cardiovascular tissue. In addition, standalone catheter assemblies as well as kits comprising components of the systems described herein are provided. The systems, assemblies, methods and kits find use in a variety of different applications, including angioplasty applications or other catheter-based therapies or treatments, e.g., treatments of heart valve calcifications.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

In further describing various aspects of the invention, the systems and components thereof are described first in greater detail, followed by a review of methods of using the systems as well as kits for practicing the subject methods.

### Systems for Treating Cardiovascular Tissue

As summarized above, systems for imparting pulsatile energy to cardiovascular tissue are provided. Systems of embodiments of the invention are configured to generate and transmit pulsatile energy to a heart-tissue-conforming element for applying pulsatile energy to cardiovascular tissue. In some cases, the heart-tissue-conforming element is used to apply pulsatile energy to heart valves or features thereof or tissue supporting heart valves. In embodiments, the heart-tissue-conforming element may be configured to focus pulsatile energy on specified regions or features or aspects of cardiovascular tissue. For example, a heart-tissue-conforming element may be configured to deliver focused pulsatile energy to heart valve commissures or heart valve leaflets.

The catheter assembly and the heart-tissue-conforming element may be configured so that fluid can perfuse past the heart-tissue-conforming element while the heart-tissue-conforming element is applying pulsatile energy to cardiovascular tissue. In embodiments, the catheter assembly including the heart-tissue-conforming element is configured so that, when deployed adjacent to cardiovascular tissue, blood can perfuse past the heart-tissue-conforming element via an active or passive perfusion mechanism that is part of the catheter assembly and the heart-tissue-conforming element. Such configurations facilitate extending treatment time, increasing the amount of pulsatile energy that can be applied to cardiovascular tissue than would otherwise be available.

Systems of the invention find use in a variety of applications, including angioplasty applications or other catheter-based therapies or treatments, e.g., treatments of heart valve calcifications. In some instances, the systems find use in fracturing hardened materials, e.g., calcium deposits, embedded within cardiovascular tissue, e.g., a heart valve leaflet or heart valve commissure or other cardiovascular tissue or tissue surrounding the heart, such as pericardial calcification. Specifically, systems of the invention find use in treating cardiovascular tissue including, for example, (1) utilizing features of the heart-tissue-conforming element to seat the heart-tissue-conforming element at a desired location adjacent to cardiovascular tissue, e.g., a heart valve; (2) utilizing features of the heart-tissue-conforming element to apply pulsatile energy to cardiovascular tissue in a focused manner, i.e., where pulsatile energy is directed to specific aspects of cardiovascular tissue (in contrast with, e.g., a monolithic balloon applying substantially equal amounts of pulsatile energy to all aspects of cardiovascular tissue); and (3) utilizing features of the catheter assembly and heart-tissue-conforming element to enable perfusion past the heart-tissue-conforming element. The present disclosure describes applications of embodiments related to treating cardiovascular tissue related to, e.g., calcifications and/or depositions within cardiovascular tissue, such as heart valves or features thereof or tissue supporting heart valves or tissue surrounding the heart, such as pericardial calcification. However, the present system and teachings are not solely limited to imparting pulsatile energy to cardiovascular tissue in connection with cardiovascular tissue calcifications and may be generally applied to other applications as determined by those skilled in the art.

The systems may be used for imparting pulsatile energy to cardiovascular tissue of any number of different subjects. In some instances, the subjects are “mammals” or “mammalian,” where these terms are used broadly to describe organisms which are within the class mammalia, including the orders carnivore (e.g., dogs and cats), rodentia (e.g., mice, guinea pigs and rats), and primates (e.g., humans, chimpanzees and monkeys). In some instances, the subjects are humans.

Aspects of the systems include a console assembly comprising a potential source, a manifold assembly operably connected to an output of the console assembly, wherein the manifold assembly comprises an oscillator configured to generate pulse energy from energy transmitted from the potential source and a catheter assembly operably connected to an output of the manifold assembly. Catheter assemblies of the present invention include a connector operably connecting the catheter assembly to the manifold assembly and configured to transduce a first pulse energy generated by the manifold assembly to a second pulse energy, a catheter comprising a fluidic passage operably connected to the output of the connector and configured to transmit the second pulse energy and a heart-tissue-conforming element configured to receive the second pulse energy transmitted through the fluidic passage of the catheter to apply pulsatile energy to cardiovascular tissue. Additionally, in certain embodiments, the heart-tissue-conforming element is configured to engage heart valve tissue. In other embodiments, the heart-tissue-conforming element comprises a plurality of distal balloons arranged circumferentially around a rigid distal region of the catheter. In some embodiments the catheter assembly is configured to allow fluid to perfuse past a distal region of the catheter. These components and configurations thereof are now described further in greater detail below.

### Console Assembly:

Systems of the present invention include a console assembly. The console assembly, also referred to as console unit or console subsystem, is used in embodiments of systems according to the present invention to generate the required power and control for treatment of cardiovascular tissue using the system.

Embodiments of console assemblies of systems according to the present invention include a potential source. Potential sources of embodiments of the invention are configured to provide energy, which may be regulated as desired by a regulator. Any convenient potential source may be employed, where examples of potential sources include voltage sources, pressure sources, electromagnetic sources, electric field sources, chemical sources, and the like. In some embodiments, the potential source is a pressure source, where examples of suitable pressure sources include, but are not limited to: compressed gas cylinders, compressors and the like. Where desired, the potential source may be operably coupled to a regulator, which serves to modulate energy from the potential source to a suitable form so that it may be further acted upon, e.g., by an oscillator of a manifold assembly. For example, where the potential source is a high-pressure gas source, the regulator may serve to regulate the pressure of the gas to a suitable value that can be input to an oscillator. In addition to positive potential sources (e.g., high-pressure gas), potential sources of interest may also include a negative potential compared with a reference or standard potential, e.g., a potential source configured to provide a vacuum potential compared to standard atmospheric conditions.

In some embodiments, the console assembly comprises more than one potential source. In embodiments that comprise more than one potential source, the potentials supplied by each potential source may all be of the same type or may be a combination of different potential types. For example, each potential source may be a pressure source (at the same or different potential levels), or, alternatively, one potential source may be a pressure source and another potential source may be a voltage source.

In embodiments, console assemblies may further comprise one or more regulators (i.e., power regulators), an output port and a controller. With respect to power regulators, as described above, in embodiments, the potential of the potential source may be regulated from a first, input potential, to a second potential, e.g., a potential that is suitable for transmitting to an oscillator of the manifold assembly and ultimately for treatment of cardiovascular tissue. The potential of the potential source may be regulated to a pre-determined value, a user-set value or may be adjusted according to a variety of feedback inputs that occur during treatment. In some cases, the potential of the potential source may be dynamically regulated based at least in part on conditions related to treatment involving imparting pulsatile energy to cardiovascular tissue, e.g., based on changes in tissue compliance during treatment, as described below. In some cases, the potential of the potential source may be regulated in real time or substantially in real time. In certain embodiments, the potential of the potential source may be adjusted to an optimal value for a certain treatment. For example, the potential of the potential source may be adjusted to an optimal value for treatment of a diseased heart valve, such as a heart valve with calcifications on or around a valve commissure. In some cases, one or more inputs from one or more of the console assembly, the manifold assembly, the catheter assembly or from a source external to the system (e.g., other measurements regarding a subject, such as imaging of the subject) may be used to determine an optimal treatment condition, e.g., output potential of the potential source appropriate for the desired treatment, and then to adjust to that condition.

In embodiments that comprise a regulator (i.e., a power regulator or potential regulator) configured to regulate the potential of the potential source, such a regulator may be a passive (i.e., preset or user-adjusted regulator) or an active regulator (i.e., a regulator that is controlled with, for example, an electrical impulse or other dynamic signal from, e.g., a controller). Regulators of interest may comprise regulators typically used for fluidic regulation such as a directional or diaphragm valve, electrical regulation such as a voltage regulator, optical power regulation or the like. In embodiments that comprise more than one potential source, the potentials of the various potential sources may be regulated together or separately.

In embodiments, the regulated and/or unregulated potential (i.e., potential energy) from the potential source is output through an output port operably coupled to the manifold assembly of the system. Any convenient output port, such as commercially available connectors, such as pneumatic, hydraulic, electrical or optical connectors, may be employed in embodiments. In certain instances, the unregulated or regulated potential energy may be converted to another energy form prior to or, in some cases, after the energy is passed or otherwise transmitted to the manifold assembly.

In some cases, the console assembly may include more than one physically separate or connected units, i.e., each, a console unit, that may be operably interconnected (e.g., electrically, fluidically, using radio frequency (RF) or the like). That is, the console assembly may comprise a unitary assembly or two or more distinct, operably connected units.

In some instances, at least some of the console assembly components are present in a unit that is configured to be hand-held or manipulated, e.g., moved, by hand. While the form factor of such a unit may vary as desired, in some instances, such units may be configured substantially as a rectangular box having height ranging from to 100 cm, such 20 cm to 30 cm, width from 5 to 100 cm, such as 10 to 20 cm and depth ranging from 10 to 100 cm, such as 20 to 30 cm, and a mass ranging from 1 to kg, such as 5 to 8 kg.

In an embodiment, a console assembly may include a first console component that houses a potential source and regulator and actuator for the pressure source, e.g., a manipulatable button. The console assembly may include an electrical connector for providing electrical connection to various other components of the system, as desired. For example, an electrical connector may be used to receive data regarding a position and/or configuration of the heart-tissue-conforming element, such as a location or orientation vis-à-vis a heart valve undergoing treatment or pressure or volume measurements, and to provide power to sensors configured to collect such data regarding treatment using the system.

In some instances, at least some of the console assembly components are present in a mountable unit that is configured to be positioned or fixed on or proximal to an operating table near a subject, i.e., a patient, so that an operator, e.g., a physician, does not need to physically interact with the console assembly (for example, the operator does not need to be physically present in an operating room and can communicate with the system via a remote control at a distance) to treat the subject. In such instances, the mountable unit is designed to be easily clamped, fixed, or independently stable on, or proximal to, the operating table and can be operated by a distal control unit (as described below in connection with the robotic control unit depicted in FIG. 16). In such instances, the mountable unit may include a communicator that provides for communication between the console assembly and the distal control unit, which may be implemented by any desired hardware and/or software configuration and may be configured to communicate using wired or wireless protocols.

Console assemblies and/or potential sources thereof that are employed in systems of the invention may be configured to be reusable or single use, as desired. Console assemblies employed in systems of the invention may be configured to receive a sterile sleeve such that the console assembly may be used while not contaminating a sterile field of the operating room. Further details regarding aspects of console units, potential sources, regulators, etc., that may be employed in embodiments of the present invention are provided in United States Published Patent Application Publication No. 20200046949 as well as pending PCT Application Serial No. PCT/US2020/055458 as well as U.S. Application No. 63/274,832; the disclosures of which are herein incorporated by reference.

### Controller:

Embodiments of the console assembly of systems according to the present invention include a control subsystem also referred to as a controller or control assembly. Embodiments of systems may utilize a controller to control the amount and duration of energy transmitted to tissue, i.e., cardiovascular tissue such as a heart valve. In some instances, embodiments of systems may utilize a controller to measure the effect of treatment on cardiovascular tissue, such as a degree of disruption of calcified tissue, e.g., cardiovascular tissue compliance, as described in detail below. In still other instances, embodiments of systems may utilize a controller to measure or control a perfusion mechanism, e.g., to control perfusion of fluid, e.g., blood, past a heart-tissue-conforming element present in a subject.

In embodiments, the control subsystem may be connected to, receive information from, and/or adjust (i.e., control) aspects of one or more of the console assembly (such as a pressure source or a regulator), the manifold subsystem (such as an oscillator) or the catheter assembly (such as a heart-tissue-conforming element). The control subsystem may also be configured to receive information from, and/or control, external systems such as an electrocardiogram (ECG), an intravascular or external pressure monitor, a blood volume sensor, patient vitals sensors or an imaging subsystem such as an imaging subsystem utilizing light, fluoroscopy, intravascular ultrasound (IVUS) or optical coherence tomography (OCT) or other available imaging techniques. In certain embodiments, such imaging subsystem (e.g., an imaging subsystem utilizing light, fluoroscopy, intravascular ultrasound (IVUS) or optical coherence tomography (OCT) or other available imaging techniques), or aspects thereof, may be integrated in the catheter assembly, e.g., integrated into the catheter of the catheter assembly, and the control subsystem may be configured to receive information from such a catheter-integrated imaging system. Further, the control subsystem may comprise multiple control units interconnected such that one or more of the units synchronize and communicate with each other.

In some cases, the control subsystem (or control units that comprise the control subsystem) may be configured to communicate with components of the system such that energy transmitted via the catheter assembly, including via the heart-tissue-conforming element, is appropriate, i.e., appropriate for a particular treatment involving applying pulsatile energy to cardiovascular tissue. In other embodiments, the control subsystem (or control units that comprise the control subsystem) may receive information, e.g., data signals, from sensors regarding the status of a cardiovascular tissue treatment, such as, for example, a heart valve delivery treatment where sensors provide information regarding location (e.g., location of a replacement heart valve), a deployment percentage (e.g., deployment of a replacement heart valve), valve opening amount, nominal characteristics (e.g., features of replacement heart valve), eccentricity of the valve, paravalvular leakage, surrounding pressure on the valve, proximal and/or distal pressure, position of the valve in the annulus and the like.

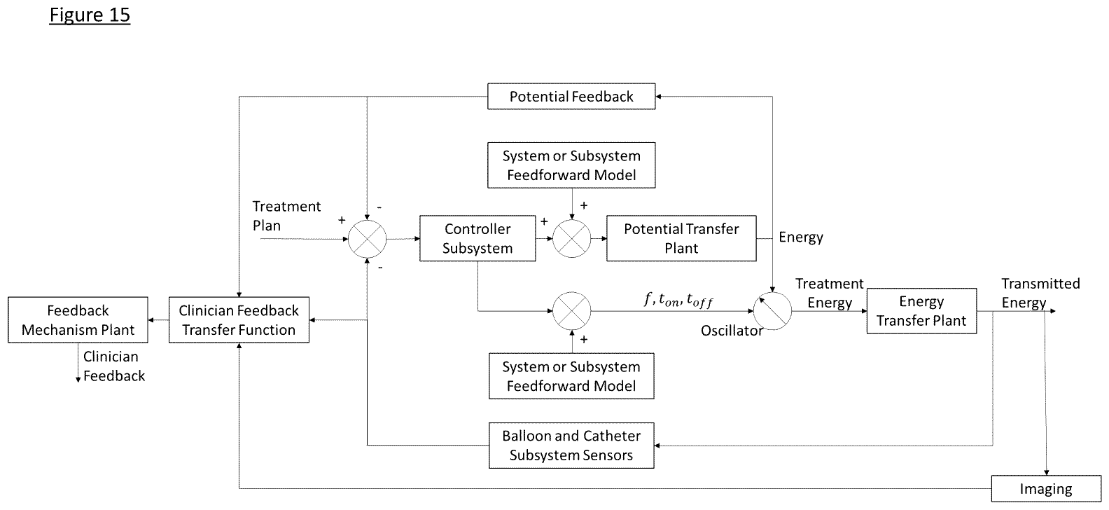

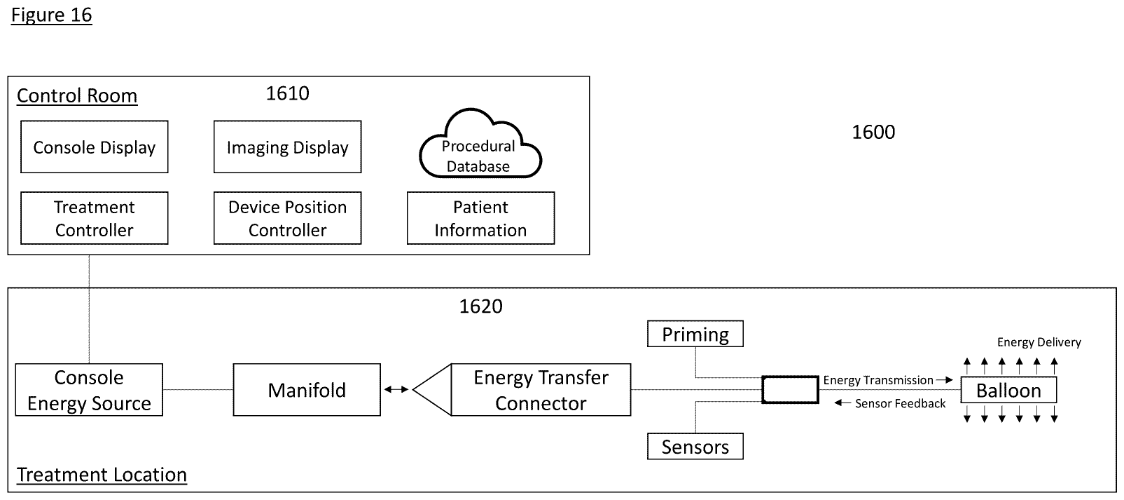

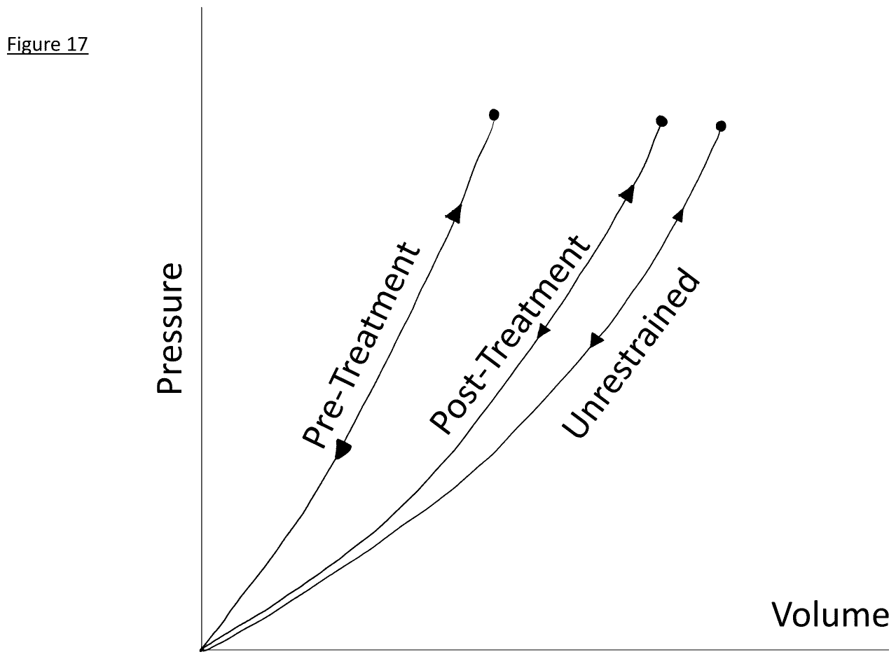

In an embodiment, the controller is configured to receive a treatment plan, i.e., control instructions related to a specific treatment for a specific treatment of a subject. A treatment plan may include, for example, a specified potential amount, a frequency or duty cycle of the oscillator. In addition, a treatment plan may include information about the type of heart-tissue-conforming element to be employed, such as a size or orientation. Additional details regarding aspects of a controller for implementing a treatment plan are set forth below in connection with exemplary control loop depicted in FIG. 15. Further details regarding treatment plans, control systems and updating the behavior of catheter-based procedures based on data collected about the procedure are described in U.S. Application Ser. No. 63/346,704 titled “Systems and Methods Related to Catheter-Based Procedures” and filed on event date herewith (Attorney Docket No. AVSI-005PRV); the disclosure of which is incorporated herein by reference. In an embodiment, the heart-tissue-conforming element is configured to expand upon the diastole phase of the cardiovascular tissue. That is, when the cardiovascular tissue, e.g., the cardiovascular tissue of a subject, on which the heart-tissue-conforming element is applied, enters a diastole phase of the cardiac cycle, the heart-tissue conforming element is expanded. In other embodiments, the heart-tissue-conforming element is configured to expand upon the systole phase of the cardiovascular tissue. That is, when the cardiovascular tissue, e.g., the cardiovascular tissue of the subject, on which the heart-tissue-conforming element is applied, enters a systole phase of the cardiac cycle, the heart-tissue conforming element is expanded. By expanded, it is meant that energy is transmitted to the heart-tissue-conforming element. In some cases, expanding the heart-tissue-conforming element comprises expanding an aspect of the heart-tissue conforming element, such as, for example, expanding one or more mid-radius balloons of the heart-tissue conforming element. For example, in some cases, expanding the heart-tissue-conforming element comprises applying pressure to, or injecting fluid into, a balloon of the heart-tissue-conforming element, increasing its volume, including, for example, expanding an external diameter of the heart-tissue-conforming element. By systole or systole phase or systole part, it is meant the part of a cardiac cycle during which the cardiovascular tissue contracts (e.g., in some cases, when heart chambers contract after refilling with blood). By diastole or diastole phase or diastole part, it is meant the part of a cardiac cycle during which the cardiovascular tissue relaxes (in contrast with contracting) (e.g., in some cases, when heart chambers relax and refill with blood after emptying during the systole phase).

In other embodiments, the heart-tissue-conforming element is further configured to relax upon the systole phase of the cardiovascular tissue. That is, when the cardiovascular tissue, e.g., the cardiovascular tissue of a subject, on which the heart-tissue-conforming element is applied, enters a systole phase of the cardiac cycle, the heart-tissue conforming element is relaxed. In other embodiments, the heart-tissue-conforming element is further configured to relax upon the diastole phase of the cardiovascular tissue. That is, when the cardiovascular tissue of a subject, on which the heart-tissue-conforming element is applied, enters a diastole phase of the cardiac cycle, the heart-tissue conforming element is relaxed. By relaxed, it is meant that energy is not transmitted to the heart-tissue-conforming element. In some cases, relaxing the heart-tissue-conforming element comprises collapsing an aspect of the heart-tissue conforming element, such as, for example, relaxing or collapsing one or more mid-radius balloons of the heart-tissue conforming element. For example, in some cases, relaxing the heart-tissue-conforming element comprises depressurizing fluid in or removing fluid from or allowing fluid to exit from a balloon of the heart-tissue-conforming element, decreasing its volume, including decreasing an external diameter of the heart-tissue-conforming element. In some embodiments, upon relaxing or collapsing or contracting the heart-tissue-conforming element, such as described above, radial force applied by the balloon on a heart valve may be reduced.

In embodiments, the system is configured so the heart-tissue-conforming element applies pulsatile energy to the cardiovascular tissue when the heart-tissue-conforming element is expanded. That is, in embodiments, the heart-tissue-conforming element may cycle between an expanded and relaxed state and may be further configured such that within such cycle, the heart-tissue-conforming element applies pulsatile energy to cardiovascular tissue only when expanded and not when relaxed or collapsed. In some embodiments, the heart-tissue-conforming element may be expanded and relaxed by periodically applying pressure to one or more mid-radius balloons of the heart-tissue-conforming element, and further configured so that winged balloons (i.e., balloons layered onto the external surface of the one or more mid-radius balloons) apply pulsatile energy to the cardiovascular tissue when the one or more mid-radius balloons are inflated, i.e., expanded, and do not apply pulsatile energy to the cardiovascular tissue when the one or more mid-radius balloons are deflated or depressurized.

In embodiments, the heart-tissue-conforming element may be configured to expand and relax (i.e., contract) in a manner that is synchronized with the diastole and systole phases of a cardiac cycle. Such expansion and relaxation of the heart-tissue-conforming element may be configured to facilitate stable positioning of the heart-tissue-conforming element with respect to cardiovascular tissue. That is, the expansion and relaxation of the heart-tissue-conforming element may facilitate positioning the heart-tissue-conforming element in the desired location relative to the cardiovascular tissue and further may facilitate causing the heart-tissue-conforming element to remain in a relatively fixed, i.e., stable, location and orientation, relative to the cardiovascular tissue. In some cases, the heart-tissue-conforming element is configured to expand and contract in a manner that emulates the behavior of a heart valve.

In embodiments, the expansion and/or relaxation of the heart-tissue-conforming element in conjunction with the systole and/or diastole phases of the cardiovascular tissue, may be determined, i.e., controlled, based on pressure signals, i.e., the results of pressure sensors, such as, for example, an intravascular pressure monitor or pressure sensors measuring pressure across the heart-tissue-conforming element (i.e., a pressure gradient between a proximal and distal location relative to the heart-tissue-conforming element), or may be determined based on electrocardiogram (ECG)-, i.e., the results of performing an electrocardiogram on the cardiovascular tissue. In other embodiments, the expansion and/or relaxation of the heart-tissue-conforming element in the systole or diastole phases of the cardiovascular tissue may be determined, i.e., controlled, based on results of a blood volume monitor or the results of an imaging system. In embodiments, systems further comprise a controller, such as described above, configured to cause the heart-tissue-conforming element to expand or contract in systole and/or diastole phases of the cardiovascular tissue. In such embodiments, the controller may be configured to expand or relax the heart-tissue-conforming element based on input from at least one of: results of an electrocardiogram, an intravascular pressure monitor, a blood volume monitor or an imaging system. In other cases, the controller may be configured to expand or relax the heart-tissue-conforming element based on a pre-determined configuration, such as, for example, at a specific frequency, such as a frequency corresponding to an induced cardiac cycle. That is, the controller may be configured to expand or relax the heart-tissue conforming element according to certain pre-determined pattern, as, for example, may be programmed in advance for operating the controller. In some embodiments, the controller may be configured to expand the heart-tissue-conforming element between 40 beats per minute to 150 beats per minute over a period of time.

In embodiments, a controller may be configured to provide feedback to an operator of a system of the present invention in any convenient manner. In some cases, the controller is configured to provide tactile feedback to an operator by, for example, vibrating. For example, the controller may be configured to cause a handle or other interface with an operator of a system to vibrate upon and relevant change or determination, such as measurement of a sensor, for example, changes in compliance of the cardiovascular tissue. Such tactile feedback may be used in connection with indicating to an operator of an embodiment of a system to change a configuration of the system.

### Manifold Assembly:

Systems of the present invention include a manifold assembly. The manifold assembly, also referred to as manifold unit or manifold subsystem, is used in embodiments of systems according to the present invention to receive energy transmitted from the potential source of the console assembly and to transmit such energy to the catheter assembly. In embodiments, the manifold assembly comprises an oscillator configured to generate pulse energy from energy transmitted from the potential source. In such instances, the oscillator is used to modulate a magnitude and timing of the potential energy from the potential energy source in order to provide for the desired energy for use in applying pulsatile energy to cardiovascular tissue via the heart-tissue-conforming element.

In embodiments, the manifold subsystem includes an input connection to the console assembly, one or more oscillators and an output connection to the catheter assembly. In some embodiments where the console assembly comprises one or more console units, as described above, the input connection comprises an input connection to one or more of the console units of the console assembly. As described above, in embodiments, the manifold subsystem is configured to receive energy from the console assembly and output energy to the catheter assembly. The manifold subsystem may be configured to receive various forms of potential energy from the one or more console units (e.g., a voltage potential, an electromagnetic potential, a pressure potential or the like) and to distribute that energy to one or more oscillators in the manifold assembly. Any convenient input and output connections, such as commercially available connectors, such as pneumatic, hydraulic, electrical or optical connectors, may be employed in embodiments.

In certain instances, the manifold assembly may receive potential energy such as from one or more console units of the console assembly and distribute that potential energy to one or more oscillators in the manifold assembly. In some cases, there is a one-to-one correspondence between console units and oscillators in the manifold subsystem. In other cases, a single console unit may deliver energy to one or more oscillators. In still other cases, one or more console units may deliver energy to a single oscillator, e.g., for example, such that the potential energies of the one or more console units are combined in a single oscillator.

In embodiments, energy transmitted to the manifold system oscillator comprises a regulated or unregulated fluid under pressure. The oscillator may be actuated to output a pulsatile and/or a static pressure output. In certain embodiments where the potential energy transmitted by the console assembly is a regulated or unregulated fluid under pressure, the oscillator may comprise a solenoid valve. Such solenoid valve may comprise, for example, a two-position, three-way, normally closed solenoid valve. In such instances, the solenoid valve is configured to receive the high-pressure regulated or unregulated fluid. Such a solenoid valve may be configured to have two modes, an “on” mode and an “off” mode. Such a solenoid valve may be configured to have three ports: a port operably connected to the high-pressure regulated or unregulated fluid (i.e., an input port), a port operably connected to, ultimately, a catheter assembly (i.e., a first output port), and an exhaust port (i.e., a second output port). The solenoid valve may be configured such that when turned on (i.e., in an “on” mode), the valve allows the high-pressure regulated or unregulated fluid to be transmitted, i.e., transmitted downstream in the system, such as transmitted to the catheter assembly of the system. The valve may be further configured such that when turned off (i.e., in an “off” mode), the solenoid changes, i.e., reverses, the connected ports such that the distal side of the valve is exhausted (e.g., exhausted to atmosphere or vacuum). That is, in the “off” mode, the first output port may be connected to the second output port, thereby exhausting high pressure fluid present on the distal side of the solenoid valve.

In certain embodiments, a frequency and/or duty cycle of the oscillator may be adjusted to generate the appropriate output for treatment and for the catheter assembly, including the heart-tissue-conforming element thereof. In various embodiments, the one or more oscillators of the manifold assembly may be configured to oscillate at one or more frequencies and/or duty cycles. In certain instances, an oscillator configured to deliver, for example, pulsatile intravascular lithotripsy to cardiovascular tissue may be configured to oscillate at a frequency between 0 and 50 Hz, such as 1-10 Hz or 10-Hz or 21-30 Hz or 31-40 Hz or 41-50 Hz, and a duty cycle between 10% and 90%, such as 10% or 20% or 30% or 40% or 50% or 60% or 70% or 80% or 90%. In instances in which an oscillator is configured to use fluid pressure to deliver pulsatile pressure pulses for a treatment involving enabling vessel perfusion of cardiovascular tissue, the oscillator may be oscillated at a frequency between 0.25 Hz and 5 Hz, such as 1 Hz or 2 Hz or 3 Hz or 4 Hz or 5 Hz, and a duty cycle between 10 and 90%, such as 10% or 20% or 30% or 40% or 50% or 60% or 70% or 80% or 90%. In instances in which an oscillator is configured to deliver pulse energy comprising an optical or high voltage source, the oscillator may be oscillated at a frequency of 0.1 Hz to 1 GHz such as 1 Hz or 2 Hz or 3 Hz or 4 Hz or 5 Hz or more and a duty cycle between 0.0001% and 90% such as 0.001% or 0.01% or 0.1% or 1% or 10% or 20% or 30% or 40% or 50% or 60% or 70% or 80% or 90%.

Further details regarding aspects of manifold assemblies and oscillators, etc., and components thereof, that may be employed in embodiments of the present invention are provided in United States Published Patent Application Publication No. 20200046949 as well as pending PCT Application Serial No. PCT/US2020/055458 as well as U.S. Application No. 63/274,832; the disclosures of which are herein incorporated by reference.

In certain embodiments, output from the oscillator of the manifold assembly, or, in embodiments with more than one oscillator, outputs from the various oscillators, can be transmitted to one or more locations. In other embodiments that comprise more than one oscillator, the oscillators can be synchronized with each other, e.g., such that pulsatile energy transmitted from each oscillator is synchronized as desired, e.g., in terms of magnitude, frequency, phase, duty cycle, etc. In other embodiments with one or more oscillator, the oscillators can be synchronized with external factors or systems or sensors such as, for example, the results of an electrocardiogram (ECG), or can be adjusted based on feedback from the controller or other subsystems (e.g., such as volume or pressure measurements that originate from the catheter assembly, such as volume or pressure measurements detected by sensors present on the heart-tissue-conforming element or the catheter). In other embodiments, the one or more oscillators may be controlled based on signals from a heart valve delivery system (e.g., a transcatheter aortic valve replacement (TAVR) delivery system) such as data regarding the location, valve deployment percentage, valve opening amount, nominal characteristics, eccentricity of the valve, paravalvular leakage, surrounding pressure and/or stress on the valve, proximal and/or distal pressure, position of the valve in the annulus and the like. That is, a controller may be configured to adjust the behavior of an oscillator based at least in part on such data.

In other embodiments, the manifold assembly may further comprise multiple inlet sources (e.g., connections to console units), a manifold encasement (e.g., a housing for the manifold assembly), a multitude of oscillators, oscillator connection points (e.g., for transmitting energy from the oscillatory to the catheter assembly), controller connection points (e.g., for transmitting input from sensors within and/or external to the system) and a user feedback and/or control area (e.g., for a user to adjust the operation of the system).

In the various embodiments described, the manifold assembly, like the console assembly, may be configured to be disposable or reusable. In cases where the manifold assembly (or console assembly) is reusable and could contact a patient area, such assembly can be configured to be covered in a disposable, sterile sleeve or bag. In certain embodiments, the manifold assembly can be configured as a part of the console assembly (i.e., such that components of the console assembly and manifold assembly are held within a single common housing). In other embodiments, the manifold assembly can be configured in the form of a handle such that an operator of the system may hold the manifold assembly during use or therapy.

In some embodiments, components of the control subsystem or controller, as described above, can be located within the manifold assembly housing and/or within the console assembly housing. In certain cases, the manifold assembly, and/or the console assembly, includes a user interface configured such that an operator of the system has access to the manifold assembly or the console assembly to start or stop treatment, adjust treatment intensities, adjust treatment modes or adjust other relevant aspects or configurations of the system.

### Catheter Assembly:

Systems of the present invention include a catheter assembly. The catheter assembly, also referred to as catheter subsystem, is used in embodiments of systems according to the present invention to receive energy transmitted from the manifold assembly and apply pulsatile energy to cardiovascular tissue. Embodiments of the catheter assembly comprise a connector operably connecting the catheter assembly to the manifold assembly and configured to transduce a first pulse energy generated by the manifold assembly to a second pulse energy; a catheter comprising a fluidic passage operably connected to the output of the connector and configured to transmit the second pulse energy; and a heart-tissue-conforming element configured to receive the second pulse energy transmitted through the fluidic passage of the catheter to apply pulsatile energy to cardiovascular tissue.

### Connectors:

As described above, in systems according to the present invention, the catheter assembly includes a connector, also referred to as proximal connector. Embodiments of connectors operably connect the manifold assembly to the catheter assembly and are configured to transduce a first pulse energy generated by the manifold assembly to a second pulse energy. In certain embodiments, one or more connectors are used to connect the manifold assembly to the catheter assembly and are configured to receive and transmit potential energy transmitted by one or more oscillators of the manifold assembly. Embodiments of connectors have a proximal connection point such that, in some instances, connectors may be releasably coupled with or fixed to the manifold assembly. Embodiments of connectors also have a distal transmission point. The connector may be configured such that the distal transmission point delivers energy transmitted from an oscillator to, e.g., a fluidic passage of a catheter and, ultimately, to a heart-tissue-conforming element for applying such energy to cardiovascular tissue. In embodiments, a connector is a component of the system located proximally in the assembled system, e.g., near the proximal end, e.g., within 1 m or closer to the proximal end.

In certain instances, energy transmitted via a connector may be in the form of a hydraulic pulse, including, for example, such as is useful in pulsatile intravascular lithotripsy, or for pulsing a heart-tissue-conforming element (i.e., a balloon thereof) to allow perfusion past the heart-tissue-conforming element to distal vessels. In other instances, energy transmitted via a connector may be in the form of an electrical impulse such that a cavitation bubble, ultrasound wave, plasma bubble or other high-pressure impulse is generated. In still other instances, energy transmitted via a connector may be in the form of an optical or laser pulse such that a cavitation bubble, ultrasound wave, plasma bubble or other high-pressure impulse is generated. In still other instances, such energy transmitted via a connector may be in the form of heat energy or energy, for example, for cryotherapy, i.e., applying heat or cold to cardiovascular tissue.

In embodiments, the connector comprises a proximal chamber and a distal chamber separated by a membrane. The volume of each of the proximal and distal chambers may vary, ranging in some instances from 0.1 mL to 100 mL, such as 1 mL to 4 mL, where in some instances the proximal and/or the distal chamber is occupied by a liquid. In each case, the proximal chamber is, ultimately, operably connected to the potential source (i.e., via an oscillator), such as, for example, a pressure source, and configured to transduce energy (i.e., pressure) to the distal chamber, via the membrane. While the form of the connector in embodiments may vary, in some instances the proximal chamber is defined by a proximal flange and the distal chamber is defined by a distal flange, where the proximal and distal flanges are positioned on either side of the membrane to define the proximal and distal chambers, which may be hermetically sealed from each other by the separating membrane.

The membrane is configured to move in response to pressure applied to the proximal chamber and, based on such movement, produce pressure in the distal chamber of the connector. The dimensions of the membrane may vary, where in some instances the membrane has an area ranging from 100 mm 2 to 5,000 mm², such as 500 mm 2 to 2,000 mm². The membrane may be fabricated from any convenient elastic (e.g., pliant) material, where in some instances the material has a hardness ranging from Shore 10 A to Shore 90 A, such as Shore 50 A, and a thickness between 0.5 mm to mm, such as 1.0 mm to 2.5 mm. Examples of suitable membrane materials include, but are not limited to: silicone, rubber and the like and in some cases may be strengthened by adding a reinforcing component, such as a braid. Where desired, a biasing component, such as a spring, may be provided to provide for a default or baseline membrane position. For example, a spring may be provided on the distal chamber side of the membrane which urges the membrane back to an initial position when force is removed from the proximal chamber side of the membrane.

In embodiments, the proximal chamber of the connector comprises a port operably connecting the proximal chamber with, ultimately, a potential source, e.g., a pressure source (i.e., via an oscillator). Similarly, in embodiments, the distal chamber of the connector comprises a port operably connecting the distal chamber with, ultimately, a fluidic passage of the catheter and, ultimately, the heart-tissue-conforming element.

Where desired, the connector may include one or more sensors, e.g., configured to provide data regarding one or more components of the system. Any convenient type of sensor may be included in the proximal connector, where sensors of interest include, but are not limited to: pressure sensors, positional sensors, displacement sensors, proximity sensors, flow sensors, temperature sensors and the like. In some instances, the connector includes a pressure sensor operably coupled to the distal chamber. In such instances, the pressure sensor may detect pressure and changes thereof in the fluid, such as liquid, in the distal chamber. When included, any convenient type of pressure sensor may be present, where examples of pressure sensors that may be present include, but are not limited to: resistive, capacitive, piezoelectric, optical, and MEMS-based pressure sensors, and the like. In some instances, the proximal connector includes a membrane positional sensor configured to provide spatial data regarding the position of the membrane at a given time, e.g., during use of the system. When present, any convenient membrane position sensor may be employed. In some instances, the membrane positional sensor is a Hall sensor, e.g., which may be employed in conjunction with one or more magnets (e.g., one or more permanent magnets or electromagnets or combinations thereof) present at one or more fixed locations relative to the membrane, such as a fixed location of the proximal connector, etc., such that one or more fixed magnets are positioned to modulate voltage of the Hall Sensor upon membrane movement. For example, in embodiments, magnets may be present on both sides of the Hall Sensor with poles facing each other. That is, in some cases, magnets may be oriented such that their poles face each other across the Hall Sensor. In some cases, one or more magnets are utilized in position sensors of embodiments of the present invention so as to generate a linear voltage or a current output, in each case, as such voltage or current output may be related to the position of the membrane. In other instances, the membrane positional sensor may be an optical sensor, electric field potential sensor, resistive sensor, magnetic sensor, angle sensor, or acceleration sensor. Further, any combination of these sensors may be used to gather positional data of the membrane or diaphragm. In cases in which a combination of membrane positional sensors is employed, e.g., to ensure sensors provide correct data across a variety of conditions, such as frequencies, sensor data may be combined through “sensor fusion” techniques, such as those known in the art. Fabrication methods of the membrane sensor may include, but are not limited to: adhesives, direct printing, welding, embedding and the like.

In instances where a connector is configured to deliver a hydraulic pulse, including, for example, such as is useful in pulsatile intravascular lithotripsy, a connector configured to deliver a low volume, high-frequency and high-pressure pulse may be used. As described above, embodiments of connectors include an entry port, which receives pulsatile and/or static energy from the manifold assembly and outputs pulsatile and/or static energy to a distal transmission point of the connector. Further details regarding embodiments of connectors which may be employed in connection with the systems described herein are provided in U.S. Application No. 63/145,641 and U.S. Application No. 63/274,832, the disclosures of which are incorporated herein by reference.

In instances where a connector is configured to pulse aspects of a heart-tissue-conforming element, for example, to allow perfusion to distal vessels (i.e., to allow perfusion past the heart-tissue-conforming element), a connector that can deliver a high-volume, low-frequency, and low-pressure pulse may be used. In certain embodiments, such connectors may be substantially in the form of a barrel syringe to deliver large volume changes. Barrel syringe connectors of interest may include a pneumatic input port, a fluid output port, a plunger, a pneumatic chamber and a fluid chamber. In certain instances, the plunger (i.e., piston) of the barrel syringe may be connected to a biasing spring, which would enable the quick return of the piston to its original state. In this condition, some or all the fluid volume would be removed from a fluidly coupled heart-tissue-conforming element, i.e., a balloon thereof. In other embodiments, a barrel syringe-type connector is selectively connected to a vacuum (i.e., the proximal side of the connector), which would facilitate evacuation of the proximal side of the barrel syringe, such that the piston of the barrel syringe-type connector would return to an original position, i.e., an equilibrium position. In embodiments of such connectors, a fluidly coupled heart-tissue-conforming element positioned at a distal region of the catheter, i.e., one or more balloons of the heart-tissue-conforming element, could be inflated or deflated rapidly. Embodiments of such connectors could have a variety of sensors monitoring the state of the heart-tissue-conforming element, such as pressure sensors, volume sensors or the like.

In other instances, a connector does not include a pneumatic or fluid connection. An embodiment of a connector that does not include a pneumatic or fluid connection may be configured to deliver pulsatile energy, including, for example, such as is useful in intravascular lithotripsy, using any convenient technique, as such techniques are known to those skilled in the art, including, for example, by generating cavitation bubbles. In some cases, such embodiments of connectors are configured to utilize cavitation-, plasma-, voltage-, light-, laser-, ultrasound- or other sonic-based technique to deliver pulsatile energy, including in connection with performing intravascular lithotripsy. Such an embodiment of a connector may include one or more electrical or optical cables that are present over the length of the system, laterally, configured to enable cavitation, ultrasonic or laser-based therapies to treat cardiovascular tissue, such as, for example, heart valves. Embodiments of a connector may transmit light or high voltage or sonic energy from the oscillator in the manifold subsystem via a proximal connection port of the connector to a distal transmission point of the connector. In such instances, the distal transmission point of the connector may include a series of electrodes or one or more laser targets configured to generate cavitation bubbles or plasma bubbles.

In instances, an embodiment of a connector may receive power from the oscillator mechanism to heat or cool fluid in the distal chamber of the connector or at the distal connection of the connector such that the temperature change induces a treatment effect on cardiovascular tissue or, for example, dissolves an active agent present on the system, e.g., coating the heart-tissue-conforming element, such as a drug or the like.

In other instances, the connector may be configured to collect and/or transmit information to the manifold subsystem from the catheter and/or the heart-tissue-conforming element. Such information may include, for example, pressure, temperature, volume, pre-loaded data (e.g., characteristics of the cardiovascular tissue prior to applying pulsatile energy), a status of a heart-tissue-conforming element, balloon status, lesion compliance, lesion opening, intravascular imaging data, drug delivery quantity, transmitted blood volume during perfusion and the like.

### Input Signal Versus Output Signal in a Mechanical System:

The described embodiments are dynamic physical systems, in which the output of the system (e.g., actual frequency, duty cycle, and amplitude) is governed by the system input (e.g., desired frequency, duty cycle, and amplitude) and the system characteristics (e.g., catheter length, friction, and flow channel lumen diameter). Embodiments of the systems are configured for generating controlled mechanical pulses, e.g., lithotripsy pulses, in a heart-tissue-conforming element (e.g., via one or more balloons thereof), such that the system output tracks, in some instances with minimal attenuation, the commanded, or desired, input signal. Signal attenuation is the reduction in amplitude in the system output versus the input because of the characteristics of the physical system. For successful treatment, minimal attenuation, in that the output pulsatile energy remains substantially similar, e.g., in terms of frequency, duty cycle and/or amplitude, to the input pulsatile energy as it propagates from the system input (e.g., potential source, oscillator and/or connector) to the system output (e.g., the heart-tissue-conforming element, or one or more balloons thereof), is required. As such, in some instances any change in frequency, if present at all, between the system input and heart-tissue-conforming element (or one or more balloons thereof) would be 30% or less, such as 20% or less or 10% or less or 5% or less. In some instances, any change in amplitude, if present at all, of the pulsatile energy between the system input and heart-tissue-conforming element (or one or more balloons thereof) would be 30% or less, such as 20% or less or 10% or less or 5% or less. In some instances, any change in duty cycle, if present at all, of the pulsatile energy between the proximal connector and distal balloon would be 30% or less, such as 20% or less or 10% or less or 5% or less.

Prior art (e.g., as described in WO 2017/168145 A1; US 2019/0000491 A1; U.S. Pat. No. 6,348,048 B1; WO 2001/010491 A2 and U.S. Pat. No. 8,574,248 B2) in this field has described methods of generating pressure pulses in an angioplasty balloon. However, because of system characteristics, the prior art either is required to proceed at low frequencies to achieve full pressure pulses or the prior art suffers from severe attenuation of the signals. In the case of low frequency pressure pulses, the balloon does not generate sufficient pulses in the vessel to achieve any improved treatment outcome. In the case of high frequency pressure pulses, the system output (i.e., the balloon pulse) does not track system inputs and/or the system output is so severely attenuated by the system characteristics that the treatment is ineffective. In other cases, the systems are designed such that the system input pressure itself is generated with such high frictional losses (e.g., a piston pump system) that high frequency input pressure pulses are attenuated prior to being transmitted to a distal region of the system.

### Connector-to-Catheter Transition Hub:

In embodiments, including, for example, embodiments comprising a plurality of connectors, the various connectors converge to one or more connector-to-catheter hub regions, in which connector outputs are coupled to transition to a flexible, elongated tube such as a catheter or fluidic channel thereof. The catheter may include one or more internal and/or external tubes (i.e., fluidic channels), into which the outputs of connectors may connect and transmit energy. In embodiments, the connectors may connect to transmit energy to such tubes or fluidic channels of the catheter via an inflation lumen. In embodiments, such tubes or fluidic channels may traverse the entire lateral length of the catheter assembly or may only traverse part of the length of the catheter assembly.

The connector-to-catheter hub may be made of any convenient material, such as polyvinyl chloride (PVC) or polycarbonate (PC) or the like, and may comprise strain relief elements, such as, for example, flexible tubing, providing flexibility in the positioning of the hub with respect to the catheter and with respect to the one or more connectors.

In embodiments, a guidewire channel may be integrated into the connector-to-catheter transition hub to enable a standard over-the-wire approach for translating the catheter assembly or the heart-tissue-conforming element thereof into a position proximal to a desired region of cardiovascular tissue. Embodiments of the system may be configured vis-à-vis a guidewire and guidewire channel such that it is an over-the-wire, rapid exchange, monorail, or the like, catheter system.

### Catheter:

As described above, in systems according to the present invention, the catheter assembly includes a catheter, also referred to as elongated catheter. Embodiments of catheters comprise a fluidic passage operably connected to the output of a connector and are configured to transmit pulse energy to the heart-tissue-conforming element (i.e., second pulse energy).

In embodiments, the catheter assembly may comprise a semi-rigid, flexible, elongated catheter that is intended to traverse from a location remote from cardiovascular tissue, e.g., from a location of the femoral or radial artery, to the site of treatment, e.g., an aortic valve or other site proximal to cardiovascular tissue. While the dimensions of the catheter may vary, in some instances, catheters of the present invention may be configured such that they are sized to fit through a 4 to 26 Fr introducer sheath, such as a 4 Fr introducer sheath, a 10 Fr introducer sheath, a 15 Fr introducer sheath, a 20 Fr introducer sheath or a 25 Fr introducer sheath or larger, as desired, for example, depending on a size of a subject's relevant arteries, a size of the heart-tissue-conforming element or other factors. In embodiments, the catheter may have an outer diameter ranging from between 1 mm to 8 mm, such as 1 mm or 1.3 mm or 2 mm or 3 mm or 4 mm or 5 mm or 6 mm or 7 mm or 8 mm. In some cases, it is desirable for the outer diameter to fall within a range of 6 Fr to 12 Fr. In some cases, it is desirable for the outer diameter to be 20 Fr or less, such as 20 Fr, 19 Fr, 18 Fr, 17 Fr, 16 Fr, 15 Fr, 14 Fr, 13 Fr, 12 Fr, 11 Fr, 10 Fr, 9 Fr, 8 Fr, 7 Fr, 6 Fr, 5 Fr, 4 Fr, 3 Fr, 2 Fr or 1 Fr or less. In other cases, it is desirable for the outer diameter to fall within a range of 2 mm to 4 mm. In embodiments, the outer diameter of the catheter may vary as needed depending upon, among other things, characteristics of the cardiovascular tissue, including a diameter of proximal luminal tissue, such as an artery or vein or other vessel. In other instances, the outer diameter of the catheter may vary depending on characteristics of the cardiovascular tissue, such as a size, or configuration, or degree or extent of calcification, of a heart valve, such as heart valve leaflets or heart valve commissures.

The outer diameter of the catheter may vary across different regions of the catheter. In some instances, the catheter may be tapered. In such instances, the catheter may comprise a taper ranging from 0.01° to 5°, such as 1° or 2° or 3° or 4°, over a region of the catheter. In some instances, different regions of the catheter may comprise different amounts of tapering, including in some instances, no tapering. The amount of tapering may vary depending on, among other things, different applications of the system, including the shape or other characteristics of the heart-tissue-conforming element, and different characteristics of the cardiovascular tissue to which the system is to be applied.

In embodiments, the length of the catheter may vary. For example, the catheter may have a length of between 50 and 300 cm, such as 50 cm or 100 cm or 145 cm or 200 cm or 250 cm or 300 cm in length.

The catheter may be fabricated from any convenient, and suitably physiologically acceptable, material, including but not limited to rubber, silicone, polyurethane, a polyimide, such as a polyimide braid, or a polyimide-type material or the like. Embodiments of the catheter may further comprise a thermoplastic jacket, for example, a polyimide. In some instances, the catheter may comprise an external coating selected based on the application of the system. For example, in some cases, embodiments of the catheter may further comprise a lubricated exterior coating. Any convenient coating that reduces friction related to the interaction of the catheter and internal luminal tissue through which the catheter is passed may be used. In some cases, such an exterior coating may comprise polytetrafluoroethylene (i.e., PTFE). In embodiments, the catheter, or aspects thereof (e.g., a distal region of the catheter), may utilize (e.g., be formed from, in whole or in part) a stainless steel or a nitinol or a hypotube, such as a laser-cut hypotube.

As described above, embodiments of catheters may have one or more channels, e.g., fluidic channels or passages, with various functions as desired. For example an embodiment of a catheter may include one or more channels for, among other things: (1) passing at least one guidewire for use navigating the catheter assembly and/or the heart-tissue-conforming element to a treatment site, i.e., proximal to specified cardiovascular tissue; (2) inflating aspects of the heart-tissue-conforming element, e.g., one or more balloons of the heart-tissue-conforming element, by, for example, propagating pressure along the fluidic passage; (3) delivering intravascular lithotripsy pulses; (3) allowing blood to perfuse (e.g., from the left ventricle to the aorta); and/or (4) making a variety of measurements such as pressure measurements, e.g., aortic or ventricular pressure, a pressure of the heart-tissue-conforming element, e.g., a pressure of a balloon of the heart-tissue-conforming element, expansion of the heart-tissue-conforming element, e.g., an expansion of a balloon of the heart-tissue-conforming element, the presence of, or a degree of, calcium cracking (i.e., disruption of calcifications), valve tissue impedance, valve compliance, commissure opening amount, replacement heart valve opening and the like.

In embodiments, a fluidic passage of the catheter configured to propagate energy (i.e., the second pulse energy), such as fluidic pressure, extends longitudinally along the catheter, for example, from a proximal region of the catheter to a distal region of the catheter. In some embodiments, a fluidic passage of a catheter is configured to receive energy transduced from one or more connectors. For example, a fluidic passage of a catheter may be configured to receive energy from each of a barrel syringe connector as well as a proximal connector comprising a proximal chamber and a distal chamber separated by a membrane, as described above, as well as in U.S. Application No. 63/274,832, incorporated herein by reference. In such an embodiment, the barrel syringe and proximal connector may be configured to work synchronously to prime the fluidic passage and generate pulse energy. In such embodiment, the barrel syringe may be used to prime the system, i.e., apply a baseline pressure to fluid present in the fluidic passage, and the proximal connector is subsequently used to provide pulsatile energy to the fluid present in the fluidic passage at the baseline pressure. In embodiments, a fluidic passage may be connected to one or more connectors, such as one, two, three, four, five, six, seven, eight, nine, ten, 20, 50 or 100 or more connectors. Such connectors, each connected to a single fluidic passage, may be of the same type and/or configuration or may differ in any relevant respect. Multiple connectors attached to a single fluidic passage may be synchronized or otherwise configured to transduce energy to the fluidic passage in any convenient manner. In other cases, the output of a single connector may be operably connected to a plurality of fluidic passages. For example, in an embodiment, the output of a single barrel syringe may be operably connected to multiple fluidic passages, such that the barrel syringe may be used to prime fluid present in each of the plurality of fluidic passages. That is, the barrel syringe may be configured to apply a baseline pressure to fluid present in each of the plurality of fluidic passages, and because one barrel syringe is operably connected to multiple fluidic passages, each of the fluidic passages is primed simultaneously. In embodiments, a connector may be operably connected to multiple fluidic passages of a single catheter or more than one catheter.

The cross-sectional flow area of an internal channel (i.e., a fluidic passage) of the catheter may vary. In some instances, the cross-sectional flow area of a fluidic passage of the catheter ranges from 0.1 mm 2 to 50 mm², such as 1 mm 2 to 5 mm². In embodiments, the cross-sectional flow area may take any convenient geometric shape, such as, for example, substantially circular or substantially elliptical or substantially triangular or substantially rectangular or substantially polygonal or combinations thereof. In some instances, a fluidic passage of the catheter extends from a proximal region of the catheter to the heart-tissue-conforming element (e.g., a balloon of the heart-tissue-conforming element). In such instances, the fluidic passage is configured to propagate energy, e.g., pressure, along the fluid passage from a proximal region of the catheter (i.e., from an output of a connector or a connector-to-catheter hub) to a distal region of the catheter, such as to the heart-tissue-conforming element operably connected to the fluidic passage. In instances, the fluidic passage may be configured to comprise a fluid. In such instances, applying pressure to the fluid enables propagation of pressure along the fluidic passage. Any convenient fluid may be used, for example, a saline solution with or without a contrast fluid.

At a distal region of the catheter assembly, one or more channels of the catheter (e.g., fluidic passages) that extend the length of the catheter may be split or manifolded or otherwise separated such that the one or more channels are directed to, i.e., routed to, their respective destinations, e.g., balloons, electrodes, targets, sensors, ports, etc.

In certain embodiments, the catheter is configured to pass an over-the-wire guidewire such that the catheter assembly, and heart-tissue-conforming element thereof, can be guided to the correct anatomical location. Guidewires of interest may comprise any convenient, commercially available guidewire, such as a standard 0.014″ or 0.035″ or other sized guidewire, as desired. Embodiments may comprise a guidewire lumen within the catheter that is configured to receive such a guidewire. The catheter assembly may be configured such that the guidewire lumen may be located axial to the catheter or may be located on one side of the catheter. In embodiments, the guidewire lumen is configured so that the guidewire present in the guidewire lumen of the catheter can traverse longitudinally relative to the catheter, thereby guiding the catheter, and heart-tissue-conforming element, to a treatment site. In some cases, the guidewire is configured to extend beyond the distal end of the catheter, i.e., the catheter may be configured to include both guidewire entrance and exit ports.

In embodiments, the catheter assembly, including in some cases, the catheter of the catheter assembly, comprises an integrated imaging subsystem. In some embodiments, the imaging subsystem is a light-, fluoroscopy-, ultrasound-, or OCT-based imaging subsystem or an imaging subsystem based on other available imaging techniques. Imaging subsystems of interest may utilize one or more such imaging modalities. Such an integrated imaging subsystem may be used to confirm that an embodiment of a system of the present invention is applied to a desired anatomical region or feature of the subject. Such an integrated imaging subsystem may be configured to generate images of, for example, luminal tissue of a subject. Such an integrated imaging subsystem may enable visualization of the location of aspects of the system, such as the heart-tissue-conforming element, relative to aspects of a subject's anatomy. In embodiments, the integrated imaging subsystem may be further integrated with the control subsystem of the device, as described above. For example, the control subsystem of the device may receive imaging data from the imaging subsystem and/or the control subsystem may control aspects of the imaging subsystem such as instructing the imaging subsystem to initiate imaging or toggle the imaging subsystem between different imaging modalities, e.g., ultrasound versus OCT, or direct the imaging subsystem towards different fields of view. In embodiments, imaging subsystems may comprise one or more ultrasound probes, transducers and receivers, as well as associated electronics. In embodiments, imaging subsystems may comprise radiopaque labels present at designated locations on the catheter assembly, e.g., the catheter or the heart-tissue-conforming element. In embodiments, imaging subsystems may comprise lighting elements and light detectors or cameras and associated electronics.

### Perfusion Mechanisms:

In some embodiments, the catheter assembly may be configured to allow fluid, e.g., blood, to perfuse past the catheter assembly, i.e., past the heart-tissue-conforming element, using an active and/or a passive perfusion mechanism. That is, the catheter assembly may be configured so that upon positioning the catheter assembly, including the catheter and the heart-tissue-conforming element proximal to cardiovascular tissue, e.g., for applying treatment to cardiovascular tissue, such as in an artery of a subject, the catheter assembly is configured to allow blood to perfuse past the catheter assembly, including, in particular, past the heart-tissue-conforming element of the catheter assembly, i.e., that might otherwise block or impede the flow of blood to distal vessels. For example, the catheter assembly may be configured to allow blood to perfuse from the left ventricle into the proximal aorta of a subject when the heart-tissue-conforming element is present near the aortic valve.

A region of the catheter, past which a perfusion mechanism enables blood to flow, is referred to as a distal catheter perfusion section. The remaining part of the catheter that is not part of the distal catheter perfusion section, i.e., that part of the catheter that does not include a perfusion mechanism, is referred to as a proximal catheter section. In embodiments, the distal catheter perfusion section may have any convenient length, such as, for example, a length between 5 mm to 150 mm, such as mm or 10 mm or 40 mm or 100 mm or 150 mm. The length of the distal catheter perfusion section may vary depending, for example, on the length or other features of the heart-tissue-conforming element. In embodiments, the distal catheter perfusion section and the proximal catheter section may comprise two separate components that are attached together to form the catheter assembly. Such a configuration may ease changing the heart-tissue-conforming element, attached to the distal catheter perfusion section, from application to application. In embodiments, the catheter assembly may be configured such that the distal catheter perfusion section connects to the proximal catheter section with an atraumatic tapered section or a step.

Active perfusion refers to an active mechanism for pulling or pushing or otherwise displacing fluid, e.g., blood, from one region to an another, i.e., past the distal catheter perfusion section and past a heart-tissue-conforming element. Passive perfusion refers to a mechanism configured to enable fluid, e.g., blood, perfusion based on, for example, pressure gradients such as existing pressure gradients created by, for example, pressure applied by cardiac contractions across different tissue regions, e.g., internal to and external to a heart chamber. That is, passive pressure gradients may be established by creating a fluid path between an existing high-pressure region and an existing lower pressure region and allowing fluid flow based on such pressure differential. For example, fluid flow may be generated via a passive mechanism by leveraging an existing pressure gradient in cardiovascular tissue such as a pressure gradient generated by the left ventricle into the aorta. Active-passive perfusion refers to a combination of an active mechanism and a passive mechanism such that a portion of the energy urging perfusion, i.e., flow of fluid past the heart-tissue-conforming element, is generated by an active mechanism and a portion is generated by passive pressure gradients.

In embodiments, a distal region of the catheter, at or near the distal end of the catheter, i.e., the distal catheter perfusion section, is configured such that a portion of its internal cross-sectional area is dedicated to allowing blood to perfuse from a relatively distal location of the catheter assembly to a relatively proximal location of the catheter assembly, e.g., from the left ventricle to the aorta, past the heart-tissue-conforming element. In such an embodiment, the distal catheter's cross-sectional area that is configured to enable blood to flow (i.e., the distal catheter perfusion section), in some cases, may be between 1 mm 2 and 50 mm², such as 1 mm 2 or 5 mm 2 or 12 mm² or mm 2 or 20 mm 2 or 30 mm 2 or 40 mm 2 or 50 mm².

In some cases, a distal region of the catheter may comprise a tip attached to the distal region of the catheter that is configured to provide an opening (i.e., a distal catheter perfusion entrance port) for blood to enter the distal catheter perfusion section and, also, is shaped or otherwise configured to reduce any trauma to surrounding tissue as the catheter tip comprising the distal catheter perfusion entrance port, as well as the catheter and heart-tissue-conforming element, traverses through, for example, blood vessels, and across, for example, a heart valve, to reach a location proximal to cardiovascular tissue that is the subject of treatment.

In embodiments, the catheter assembly may comprise one or more ports (i.e., porting holes) configured to allow blood to enter and exit, i.e., perfuse to, for example, the aorta from a distal region of the catheter assembly. For example, such ports may be present on one or both of the catheter and the heart-tissue-conforming element. Such ports may be arranged in perfusion entrance and exit zones. Ports of the perfusion entrance and exit zones may be configured uniformly over a region of the catheter assembly or clustered near important branches, i.e., anatomical branches expected to be proximal to the catheter assembly when the catheter assembly is positioned to apply treatment to cardiovascular tissue, such as, for example, the coronary ostia. Embodiments may comprise any convenient number of ports in each of the perfusion entrance and exit zones and such may vary. For example, embodiments may comprise between 1 and 5,000 or more ports in each of the perfusion entrance and exit zones, such as one, two, three, four, five, six, seven, eight, nine, ten, 11, 12, 13, 14, 16, 17, 18, 19, 20, 100, 200, 500, 1,000, 2,000, 3,000, 4,000 or 5,000 or more ports. In embodiments, the number of, and configuration of, ports comprising the perfusion entrance and exit zones may differ. In embodiments, the perfusion entrance and exit zones may comprise a number and/or configuration of ports such that the ports do not introduce structural weaknesses into the catheter assembly. Ports may be configured to have any convenient diameter, cross-sectional shape and arrangement on the catheter or heart-tissue-conforming element, as the case may be, and such may vary. For example, embodiments may comprise ports with substantially circular cross sections with diameters ranging from 0.01 mm to 3 mm such as 0.05 mm or 0.5 mm or 1 mm or 2 mm or 3 mm. In some cases, it is desirable for the ports to have a diameter of 1 mm or greater in order to permit sufficient perfusion of fluid through the perfusion entrance and/or exit zones. In embodiments, the ports of the perfusion entrance and exit zones may be configured to reduce or minimize the amount of internal resistance encountered by fluid perfusing from the perfusion entrance zone to the perfusion exit zone. In some cases, the one or more ports of the perfusion exit zone are configured so that the total flow area (i.e., cross sectional area available for fluid to perfuse) matches or exceeds that of the perfusion entrance zone. The arrangement of the ports on the catheter and/or heart-tissue-conforming element may assume any convenient pattern. The number of ports, their diameters, and their arrangement on the catheter assembly may be selected to ensure that the catheter retains its desired structural characteristics, notwithstanding the presence of ports. In some embodiments, specifically, the number of ports, their diameters and their arrangement on the catheter assembly may be selected to ensure that the catheter maintains sufficient stiffness for applying pulsatile energy to cardiovascular tissue (i.e., via the heart-tissue-conforming element) as well as enabling sufficient blood to perfuse past the heart-tissue-conforming element to adequately perfuse distal vessels. In embodiments, ports may be formed using any convenient technique capable of forming ports with the desired characteristics. For example, in some cases, ports may be formed using laser, i.e., embodiments may be laser ported. In other cases, ports may be skived, i.e., by using a skiving technique on an embodiment in order to generate the desired ports. In still other cases, ports may be formed using a braiding configuration, i.e., embodiments may be formed in part using a braiding technique that forms the desired ports.

As described above, in embodiments, the catheter assembly is configured such that a portion of the catheter assembly is configured to retrieve blood (e.g., from a relatively distal region of the catheter) and another portion of the catheter may be configured to deliver blood (e.g., to a relatively proximal region of the catheter). In an embodiment, a continuous reciprocating motion of a barrel-like syringe connector may be configured to pull vacuum (or otherwise create a low pressure region) on an inlet (i.e., blood retrieval) port and push fluid (i.e., blood perfusion) to ports in the catheter assembly to maintain blood flow, i.e., to maintain blood perfusion to distal vessels notwithstanding the presence of the catheter assembly. For example, in an embodiment, two one-way valves may be configured to ensure that one full stroke of reciprocating motion of the connector, e.g., a barrel-like syringe connector, pulls blood from one area of, e.g., a vessel, and pushes it to another area. In some instances, active perfusion may be performed multiple times to simulate the pumping motion of the heart, or, in other instances, it may be performed once to initiate a pressure gradient, e.g., a pressure gradient across a heart valve.

In some cases, the perfusion mechanism, in particular, a cross-sectional area of the perfusion mechanism, as described above, comprises a valve configured such that fluid, e.g., blood, is not able to regurgitate from a proximal location (e.g., the perfusion entrance zone) to a distal location (e.g., the perfusion exit zone). Any convenient valve may be applied and may be located at any convenient region of the perfusion mechanism, such as the perfusion entrance or exit zone or within fluidic passages connecting the perfusion entrance and exit zones. In some instances, such valve may be a passive one-way valve. Such a one-way valve may be configured, for example, to open when pressure on the distal side of the valve (i.e., in a relatively distal region of the catheter assembly) becomes higher than pressure on the proximal side of the valve (i.e., in a relatively proximal region of the catheter assembly) and closes when the pressure differential reverses (i.e., when blood flow reverses). In other configurations, an oscillating balloon may be configured within the fluidic passages, or blood flow path, of the perfusion mechanism of the catheter assembly (i.e., within the perfusion cross-sectional area of the perfusion mechanism) and may be used to open and close one or more ports to allow and/or prevent blood flow through such one or more ports.

In some configurations, a surface of the internal lumen of the perfusion cross-sectional area of the catheter assembly (i.e., an internal surface of the perfusion mechanism) may be coated such that it is lubricious or prevents coagulation of blood passing through such area. In embodiments, the internal surface of the catheter may be coated with a hydrophilic or hydrophobic coating. In other instances, the internal surface of the catheter may be coated with an active agent, such as, for example, a heparinized coating.

In other configurations, the internal lumen of the perfusion cross-sectional area of the catheter may be fluidly coupled to an injection port such that the system is configured to inject a fluid such as, for example, a contrast, saline or an active agent, such as a drug, into the blood stream.

### Distal Region of Catheter:

In instances, a distal region of the catheter is configured to form a rigid structural tube that provides foundational, radial support to the heart-tissue-conforming element or components thereof, e.g., balloons or other objects that are affixed to and/or surround such distal region of the catheter. As described above, the catheter may be fabricated from any suitable physiologically acceptable material that provides desired structural rigidity, including but not limited to a polyimide, such as a polyimide braid, or a polyimide-type material and the like. For example, in some cases, the heart-tissue-conforming element may comprise one or more, e.g., four or more, balloons (referred to as mid-radius balloons, including, mid-radius balloons in a circumferentially located configuration) that surround such distal region of the catheter. When such mid-radius balloons are inflated, the outer diameter of the catheter shaft is configured to support and maintain contact with the opposing faces of the surrounding inflated mid-radius balloons without substantially deforming the outer diameter or shape of the catheter. When such mid-radius balloons are inflated to a high-pressure, and, for example, if they are non-compliant balloons, these mid-radius balloons may provide a high radial force at a larger diameter than that of the distal region of the catheter itself. Such a configuration forms a stacked structure (i.e., where mid-radius balloons are layered, or “stacked,” around the outer circumference of the catheter) as well as a collapsible structure (i.e., upon deflation of the mid-radius balloons, the mid-radius balloons collapse to a diameter substantially similar to the outer diameter of the catheter) that can be passed through narrow lumens, e.g., arteries. Additionally, such multi-layered structure (i.e., where mid-radius balloons are layered around the outer circumference of the catheter) generates a rigid mid-radius structure (i.e., a rigid structure around the outermost diameter of the structure formed from the inflated mid-radius balloons) that provides substantial radial support to elements, such as other components of a heart-tissue-conforming element, e.g., balloons, locating nubs, or other objects, e.g., heart valves, including other elements configured for performing intravascular lithotripsy, as such are known in the art, that are present on or surround such mid-radius balloons In embodiments, the rigid mid-radius structure, comprising a distal region of the catheter and one or more mid-radius balloons, is configured to provide enough structural stability and support for such elements, e.g., additional balloons, e.g., winged balloons, as described herein, to apply pulsatile energy, e.g., apply pulsatile force, to cardiovascular tissue, such as heart valve leaflets or heart valve commissures.

In some embodiments, to ensure both rigidity and flexibility of such a distal region of the catheter, the cross-sectional shape of such distal region of the catheter is configured to take the form of a specific geometric shape. The catheter may be configured such that its cross section has any convenient geometric shape, and such may vary depending, for example, on the features of the heart-tissue-conforming element as well as the structural requirements of the catheter. For example, the catheter may be configured such that its cross section takes the form of a circle, lobed-circle, ellipse, triangle, star or the like.

### Heart-Tissue-Conforming Element:

As described above, in systems according to the present invention, the catheter assembly includes a heart-tissue-conforming element. Embodiments of heart-tissue-conforming elements are configured to receive pulse energy (i.e., the second pulse energy) transmitted through the fluidic passage of the catheter to apply pulsatile energy to cardiovascular tissue.

In embodiments, the heart-tissue-conforming element may be configured to conform to any aspect of cardiovascular tissue relevant to treatment involving applying pulsatile energy thereto. In some embodiments, the heart-tissue-conforming element is configured to engage heart valve tissue, including, for example, tissue of a mitral valve, a tricuspid valve, a bicuspid valve, an aortic valve or a pulmonary valve. In certain embodiments, the heart-tissue-conforming element is configured to engage a heart valve leaflet or heart valve commissure or heart valve leaflet nodule or a heart valve annulus. In embodiments, the heart-tissue-conforming element comprises a shape that engages a heart valve leaflet or heart valve commissure or heart valve leaflet nodule or a heart valve annulus. In embodiments, the heart-tissue-conforming element comprises a shape that engages a heart valve leaflet or heart valve commissure or heart valve leaflet nodule or a heart valve annulus upon inflation or pressurization of one or more balloon elements of the heart tissue conforming element. In some cases, the heart-tissue-conforming element is configured to engage tissue supporting a heart valve. In particular, in some embodiments, the heart-tissue-conforming element is configured to engage an aortic valve, including, for example, an aortic valve leaflet or an aortic valve commissure or an aortic annulus. In other embodiments, the heart-tissue-conforming element is configured to engage an interatrial septum or an interventricular septum. In other embodiments, the heart-tissue-conforming element is configured to engage mitral valves as well as bicuspid aortic valves. In embodiments, the heart-tissue-conforming element comprises a shape that engages tissue supporting a heart valve or an aortic valve, including, for example, an aortic valve leaflet or an aortic valve commissure or an aortic annulus or an interatrial septum or an interventricular septum or a mitral valve or a bicuspid aortic valve. In embodiments, the heart-tissue-conforming element comprises a shape that engages tissue supporting a heart valve or an aortic valve, including, for example, an aortic valve leaflet or an aortic valve commissure or an aortic annulus or an interatrial septum or an interventricular septum or a mitral valve or a bicuspid aortic valve upon inflation or pressurization of one or more balloon elements of the heart tissue conforming element.

In embodiments, the heart-tissue-conforming element is located at a distal region of the catheter, such as, for example, within 20 cm or less of the distal end of the catheter, such as 1 cm or less or 2 cm or less or 3 cm or less or 4 cm or less or 5 cm or less or 6 cm or less or 7 cm or less or 8 cm or less or 9 cm or less or 10 cm or less or 11 cm or less or 12 cm or less or 13 cm or less or 14 cm or less or 15 cm or less or 16 cm or less or 17 cm or less or 18 cm or less or 19 cm or less or 20 cm or less from the distal end of the catheter. The heart-tissue-conforming element, or aspects thereof, may be bonded or otherwise affixed to the catheter using any convenient and physiologically suitable technique, such as with an epoxy, cyanoacrylate, plastic cement, solvent bonding technique, thermoplastic reflow or combination thereof, or any other suitable glue or adhesive or bonding technique.

### Mid-Radius Balloons:

In some embodiments, the heart-tissue-conforming element comprises one or more distal balloons present on a rigid distal region of the catheter. Such balloon may be referred to as a mid-radius balloon and may be configured to contact and apply pulsatile energy to cardiovascular tissue directly or indirectly via additional elements affixed to the mid-radius balloon, i.e., an exterior surface thereof, such as additional balloons, such as winged balloons, or nubs or the like, as described below, or combinations thereof; i.e., a mid-radius balloon may both contact and apply pulsatile energy directly to cardiovascular tissue and may also indirectly apply pulsatile energy to cardiovascular tissue via, for example, winged balloons attached to a rigid surface thereof. Such balloons are referred to as “mid-radius” in part because the balloons expand to a radius that extends radially beyond the outer circumference of the catheter but does not extend to such an extent that it fully engages the cardiovascular tissue, as described below; i.e., it occupies a middle radius position. In some embodiments, one or more mid-radius balloons are configured primarily to hold radial force so that the outer elements (i.e., elements attached to the outer circumference of the one or more mid-radius balloons), such as, for example, winged balloons, can deliver pulses, i.e., pulsatile energy, at a distance from the longitudinal axis of the catheter. That is, in such embodiments, the one or more mid-radius balloons may be configured to comprise a substrate on which additional elements, such as, for example, winged balloons, can deliver pulsatile energy to cardiovascular tissue. Such an approach enables the additional elements, such as winged balloons, to apply pulsatile energy at specific, targeted locations around the exterior surface of the one or more mid-radius balloons (i.e., such that pulsatile energy may not be applied to every aspect of the cardiovascular tissue to the same extent), such as, for example, targeted at heart valve commissures or leaflets.

Embodiments may comprise one to ten or more mid-radius balloons, such as one mid-radius balloon, two mid-radius balloons, three mid-radius balloons, four mid-radius balloons, five mid-radius balloons, six mid-radius balloons, seven mid-radius balloons, eight mid-radius balloons, nine mid-radius balloons or ten or more mid-radius balloons. In some cases, the heart-tissue-conforming element comprises one or more mid-radius balloons fixed to the catheter both proximally and distally (with respect to the balloons) and configured to surround, or substantially surround, the outer circumference of the catheter at a distal region, including in some cases, the distal end, of the catheter. In some cases, a single mid-radius balloon envelopes a distal region of the catheter, e.g., such that the mid-radius balloon and the catheter share a longitudinal axis.

Embodiments of the present invention may be configured to enable the heart-tissue-conforming element, or the one or more mid-radius balloons thereof, to translate into and out of a lumen of the catheter assembly, e.g., a central lumen of the catheter, e.g., present at a distal region of the catheter. That is, the heart-tissue-conforming element, or the one or more mid-radius balloons thereof, including any attachments thereto, such as winged balloons, as described below, or other features attached to or present on the one or more mid-radius balloons, e.g., the surface of such balloons, may be moved, e.g., by the control of an operator using any convenient technique, into and out of, e.g., a central lumen of the catheter. Embodiments of the present invention may be configured to enable the heart-tissue-conforming element, or the one or more mid-radius balloons thereof, to translate into and out of a lumen of the catheter assembly using any convenient technique, such as any convenient mechanical connection between the heart-tissue-conforming element and a proximal apparatus for controlling the translation of the heart-tissue-conforming element or the like. The one or more mid-radius balloons may be present within the catheter when the catheter is moved to a desired location within a lumen, such as a desired anatomical region of a subject, and the one or more mid-radius balloons may be moved out of such catheter lumen prior to inflating the one or more mid-radius balloons, i.e., prior to utilizing the heart-tissue-conforming element by inflating one or more mid-radius balloons, or other features thereof, to, for example, engage with anatomical tissue, such as a heart valve, or disrupt calcium deposits on anatomical tissue, such as a heart valve. Embodiments configured to enable the one or more mid-radius balloons to translate into and out of a lumen of the catheter assembly may provide certain advantages, including, for example: easier rewrapping of the one or more mid-radius balloons and any attachments thereto, easier folding of one or more mid-radius balloons and any attachments thereto, easier tracking of the heart-tissue-conforming element or the one or more mid-radius balloons thereof, such as tracking the location thereof, or, use of a narrower introducer to guide the heart-tissue-conforming element to the desired anatomical location.

During use of an embodiment of a heart-tissue-conforming element, initially, a mid-radius balloon of a heart-tissue-conforming element may be tightly wrapped and/or folded around the exterior surface of the catheter in order to allow passage of the catheter assembly, or a distal region thereof, through, for example, an introducer sheath and into the appropriate, desired anatomical location, e.g., near cardiovascular tissue. Following delivery to and positioning proximal to (i.e., near) the desired anatomical location, a mid-radius balloon of the heart-tissue-conforming element may be inflated with fluid via a fluidic coupling between the interior of the balloon and a fluidic passage of the catheter. Such a fluidic coupling between the interior of the balloon and a fluidic passage of the catheter may comprise one or more porting holes configured so that the interior of the balloon is in fluidic communication with the fluidic passage. Any convenient number, size and configuration of porting holes may be applied to operably connect the fluidic passage of a catheter to a mid-radius balloon. Porting holes in embodiments may be radial holes connecting, for example, an inner wall of a fluidic passage of the catheter (i.e., the surface closest to the fluidic passage's longitudinal axis) with the external wall of the fluidic passage (i.e., the surface furthest from the longitudinal axis, for example, the surface to which the mid-radius balloon is attached). Porting holes may be configured to allow the passage of fluid between the fluidic passage and a mid-radius balloon. In embodiments, a mid-radius balloon may be inflated using fluid transferred into the interior of the balloon from the fluidic passage of the catheter via the porting holes of the fluidic passage. That is, in some embodiments, pressure applied to the proximal end of the fluidic passage, propagated along the fluidic passage of the catheter, may further propagate to the mid-radius balloon via the porting holes.

The number of porting holes may vary in embodiments of the invention. Porting holes may be any convenient diameter and their diameter may vary. The arrangement of the porting holes on the fluidic passage may assume any convenient pattern. The number of porting holes, their diameters, and their arrangement on the fluidic passage of the catheter may be selected so as to ensure that the catheter retains its desired structural characteristics, notwithstanding the presence of porting holes. In some embodiments, specifically, the number of porting holes, their diameters and their arrangement on the catheter may be selected to ensure that the catheter maintains sufficient radial stiffness for supporting the expansion of the mid-radius balloon and the application of pulsatile energy to cardiovascular tissue via elements attached to the mid-radius balloon, such as, for example, winged balloons. For example, some embodiments of catheters according to the present invention may comprise between 1 and 5,000 porting holes (such as 100 or 300 or 1,000 or 3,000), with diameters between 0.1 mm and 0.5 mm (such as 0.1 mm or 0.25 mm or 0.4 mm), spaced at least 0.1 mm to 5 mm apart from one another (such as 0.5 mm or 1 mm or 5 mm).

In embodiments, systems may be configured such that the one or more mid-radius balloons of the heart-tissue-conforming element are periodically inflated and deflated. In some cases, systems are configured such that the one or more mid-radius balloons are inflated and deflated such that they mimic a cardiac cycle, such as, for example, periodically inflating and deflating the one or more mid-radius balloons once per second, i.e., at 1 Hz or at another frequency that is substantially synchronized with a cardiac cycle. In embodiments, aspects of the cardiac cycle may be determined by using sensors to measure a pressure gradient across the heart-tissue-conforming element (i.e., a gradient across a proximal and distal location of the heart-tissue-conforming element). As described further below, in embodiments, during the time in which the one or more mid-radius balloons are inflated (i.e., expanded such that they form a rigid substrate for additional elements, such as, for example, winged balloons to apply pulsatile energy to cardiovascular tissue), the additional elements, such as for example, outer radius balloons or winged balloons, are pulsed a plurality of times, such as, for example, up to 15 to 20 times, i.e., at a higher frequency than that of the one or more mid-radius balloons, such as at a frequency of, for example, 15 to 20 Hz. That is, in such embodiments, treatment (i.e., pulsatile energy) is applied when the one or more mid-radius balloons are inflated and not when the one or more mid-radius balloons are relaxed or collapsed. Such a configuration can help to prevent the occurrence of ischemia (because, for example, in embodiments, periodically deflating or relaxing the one or more mid-radius balloons promotes perfusion of blood past the heart-tissue-conforming element) and further helps to ensure that the appropriate treatment energy is being delivered to the cardiovascular tissue, such as, for example, a heart valve. In embodiments comprising more than one mid-radius balloon, the mid-radius balloons may be synchronously pressurized and depressurized, i.e., at the same time, or may be pressurized and depressurized independently and at different times; e.g., the mid-radius balloons may be inflated in a sequential manner such that the intermediate shape of the heart-tissue-conforming element as the mid-radius balloons inflate conforms to a desired shape or feature of the cardiovascular tissue that, for example, promotes alignment of the heart-tissue conforming element with the cardiovascular tissue.

Further, in embodiments, characteristics of pulsatile energy applied by one or more mid-radius balloons can change over the course of using mid-radius balloons in connection with applying pulsatile energy to cardiovascular tissue. Changes to characteristics of pulsatile energy applied by mid-radius balloons may be defined in advance, i.e., pre-programmed, using, for example, a controller, such as a pre-programmed controller, or the like, or can be changed dynamically, i.e., on the fly, based on any convenient parameter, such as, for example, sensor inputs, such as a pressure or volume measurement (e.g., a pressure or volume of a balloon) or based on other feedback signals or a user input or the like. For example, pulsatile energy applied to one or more mid-radius balloons can continuously increase or continuously decrease (i.e., the maximum or minimum or average energy applied to one or more mid-radius balloons can continuously increase or continuously decrease) or pause (i.e., the maximum or minimum energy applied to one or more mid-radius balloons can remain constant, including, for example, by applying no pulsatile energy, e.g., pressure, to the balloons for a period of time, i.e., a relaxation period). In addition, in embodiments, pulsatile energy applied to mid-radius balloons can slow down or speed up (i.e., pulsatile energy applied to mid-radius balloons can be applied at higher or lower frequencies, or in other cases, the rate of change of a maximum or minimum or average energy applied to mid-radius balloons can be increased or decreased).

In an inflated state, a mid-radius balloon of the heart-tissue-conforming element may be configured to take on any desired shape and as such may vary, depending, for example, on the intended treatment or anatomical features of the cardiovascular tissue or structural requirements of the catheter assembly. For example, a mid-radius balloon of the heart-tissue-conforming element may take on various shapes, including, for example, a sphere, Yo-Yo, oval, cylinder, peace-sign or the like. In some embodiments, a cross-sectional shape of the heart-tissue-conforming element, or one or more mid-radius balloons thereof, is substantially an oval shape. Such an oval shape may be of interest for use with bicuspid valves. In some cases, such an oval shape may be of interest for use in addressing mitral calcification. In other cases, such an oval shape may be of interest for use with both mitral valves as well as bicuspid aortic valves. In embodiments wherein the heart-tissue conforming element comprises more than one mid-radius balloon, each mid-radius balloon may take on the same shape or different shapes. By yo-yo shape, it is meant that one or more mid-radius balloons, upon inflation, form lobes or hemispheres, analogous to the two hemispherical sections of a yo-yo, e.g., the two pieces of a yo-yo joined by the axle of the yo-yo, such that they abut one another in a manner that allows cardiovascular tissue to be present between the two lobes or hemispheres. Such a yo-yo configuration may be employed to apply pulsatile energy to cardiovascular tissue present between the two hemispheres of the mid-radius balloons that form the yo-yo configuration. That is, a yo-yo configuration may be configured to allow cardiovascular tissue, such as, for example, heart valve leaflets to be sandwiched between the two hemispheres of the yo-yo configuration of mid-radius balloons, such that applying pulsatile energy to the cardiovascular tissue comprises squeezing the sandwich together, compressing the cardiovascular tissue, in a periodic manner.

In embodiments, a mid-radius balloon of the heart-tissue-conforming element may be configured so that increasing pressure applied to such balloon (e.g., via increasing pressure applied to fluid present in the balloon) increases the force applied by such balloon to cardiovascular tissue, i.e., increasing the force applied to a heart valve or heart valve leaflet or heart valve commissure. That is, applying more pressure to a mid-radius balloon of the heart-tissue-conforming element may cause such balloon to apply a greater magnitude force to nearby cardiovascular tissue. In embodiments, a mid-radius balloon of the heart-tissue-conforming element may be configured so that increasing pressure applied to such balloon (e.g., via increasing pressure applied to fluid present in the balloon) expands the volume of the balloon, including, in some cases, expanding the diameter of the mid-radius balloon. For example, increasing pressure applied to a mid-radius balloon may cause such balloon to protrude a greater radial distance from the longitudinal axis of the catheter.

In certain instances, the diameter of a mid-radius balloon, described above, of the heart-tissue-conforming element, may correspond to the amount of pressure and/or volume of fluid injected into the balloon, i.e., such balloon may be compliant or semi-compliant. In other instances, a diameter of a mid-radius balloon of the heart-tissue-conforming element may remain substantially fixed (i.e., constant) regardless of the amount of the internal pressure and/or volume of fluid injected into the balloon, e.g., additional pressure beyond a threshold amount.

Any convenient balloon may be employed as a mid-radius balloon. Suitable balloons include, but are not limited to, standard angioplasty balloons, such as compliant and non-compliant angioplasty balloons. In one embodiment, a mid-radius balloon is a composite balloon that includes two distinct layers, which layers include a non-compliant layer and compliant layer, e.g., as further described in U.S. Application No. 63/274,832, the disclosure of which is herein incorporated by reference. In one embodiment of the mid-radius balloon comprising a composite angioplasty balloon, a non-compliant balloon is covered with a compliant sleeve to achieve an “arrowed” pressure-stretch, e.g., as further described in pending PCT application serial no. PCT/US2020/055458, the disclosure of which is herein incorporated by reference. The compliant layer may be a rubber, silicone, polyurethane, or nitinol material or another material that can stretch up to 100-500% before failure, can withstand thousands of cycles (i.e., expansion/relaxation cycles or applications of pulsatile energy) before failure, and encounters minimal, if any, plastic deformation during expansion.

In embodiments, a mid-radius balloon of, or each of a plurality of mid-radius balloons of, the heart-tissue-conforming element may have any convenient length and inflated diameter, and such may vary as desired, depending, for example, on the intended treatment or anatomical features of the cardiovascular tissue or structural requirements of the catheter. In some embodiments, a mid-radius balloon of the heart-tissue-conforming element may have a length between approximately 1.5 cm and 4 cm or any variation thereof, such as 1.5 cm or 2 cm or 3 cm or 4 cm, and, upon inflation, a diameter (i.e., an unconstrained diameter) that ranges between approximately 1.5 cm and 4 cm or any variation, such as, for example, 1.5 cm or 2 cm or 3 cm or 4 cm in diameter, for the above, including, for example, a balloon with a 1.5 cm diameter tip or tips configured to enable passage (e.g., through cardiovascular tissue, e.g., luminal tissue) and a distal tip of smaller diameter (e.g., 1.5 cm or less) configured to decrease the risk of left ventricular outflow tract dilatation or dissection. In such embodiments, such balloon may extend radially from the exterior surface of the catheter by varied dimensions as well as to secure the balloon in place prior to energy administration (e.g., application of pulsatile energy). In general, in embodiments, balloons are between 3 cm and 6 cm in length, such as, for example, 3 cm or 4 cm or 5 cm or 6 cm in length.

In embodiments, a distal and/or a proximal region of a mid-radius balloon of the heart-tissue-conforming element may be configured to expand to a greater or to a lesser degree than a middle region of the balloon. That is, embodiments of such a balloon may take a substantially hourglass-like shape or oval-like shape or may be configured such that a plurality of mid-radius balloons, together, take such a shape. In general, in embodiments, a mid-radius balloon may not be symmetrical, e.g., symmetrical via all planes through its longitudinal axis or planes perpendicular to its longitudinal axis.

In other cases, a distal and/or a proximal region of a mid-radius balloon of the heart-tissue-conforming element is compliant or semi-compliant or non-compliant such that it engages with cardiovascular tissue, e.g., a heart valve leaflet or heart valve commissure, to seat itself in a desired anatomical location during treatment. In still other cases, a middle region of a mid-radius balloon of the heart-tissue-conforming element is compliant or semi-compliant or non-compliant such that it engages with cardiovascular tissue, e.g., a heart valve leaflet or heart valve commissure, to seat itself in a desired anatomical location during treatment. For example, in embodiments in which a mid-radius balloon is hourglass-shaped, proximal and distal regions of such mid-radius balloon would be compliant such that the diameters of such regions of the balloon correspond with an inflation pressure and/or volume of fluid injected into the balloon, and a middle region of the mid-radius balloon would be non-compliant.

In some embodiments, a distal and/or a proximal region of a mid-radius balloon of the heart-tissue-conforming element comprises passive features that improve the ability of the heart-tissue-conforming element to locate and hold a fixed position relative to the cardiovascular tissue. For example, embodiments comprise components such as nubs or locating elements configured to maintain the position of the heart-tissue-conforming element and/or the catheter assembly relative to a desired anatomical feature (such as relative to a heart valve leaflet or commissure) during treatment using a system of the present invention. That is, in embodiments, components such as nubs or locating elements may be located on an external surface of a mid-radius balloon and configured to conform to relevant cardiovascular tissue so as to engage with aspects of such cardiovascular tissue and substantially locate the heart-tissue-conforming element and hold it in a fixed position relative to the cardiovascular tissue. In some cases, such components may be configured so that the heart-tissue-conforming element is self-centering. For example, in some cases, outer balloons located on the mid-radius balloon may be inflated first such that they expand prior to inflation of the one or more mid-radius balloons, which, upon expansion, is guided by the outer balloons, which seat themselves in desired features of cardiovascular tissue, e.g., valve commissures, to align the heart-tissue-conforming element as desired.

In other examples, an exterior surface of a mid-radius balloon of the heart-tissue-conforming element, i.e., a surface that interfaces with cardiovascular tissue, may be rough, or otherwise comprise features to diminish the smoothness and/or slipperiness of the surface, in order to facilitate having the surface of the heart-tissue-conforming element maintain a fixed position, rather than sliding into different positions or orientations, relative to the cardiovascular tissue.

In embodiments, the heart-tissue-conforming element may comprise a series of mid-radius balloons located circumferentially around the catheter, i.e., one or more mid-radius balloons, as described above, may comprise such circumferentially located configuration of mid-radius balloons. In such a circumferentially located configuration of mid-radius balloons, the mid-radius balloons may be fused to each other as well as to a distal region of the catheter at their respective attachment points, e.g., at a point on the circumference of each balloon. Such circumferentially located mid-radius balloons may be fused to each other and/or to the catheter using any convenient and physiologically suitable bonding technique, such as with an epoxy, cyanoacrylate, plastic cement, solvent bonding technique, thermoplastic reflow or combination thereof, or any other suitable glue or adhesive or bonding technique.

In some instances, mid-radius balloons comprising a series of circumferentially located balloons of the heart-tissue-conforming element are non-compliant, such that, when inflated, the diameter of such mid-radius balloons is fixed, known, and structurally stable. Additionally, in embodiments, when such mid-radius balloons are inflated, the heart-tissue-conforming element may be configured so that such balloons do not occupy the full volume between the overall outer diameter of the heart-tissue-conforming element and the catheter. That is, for example, in a cross section of the heart-tissue-conforming element, such a collection of circumferentially located mid-radius balloons do not fully consume the cross-sectional area between the exterior surface of the heart-tissue-conforming element and the exterior surface of the catheter. Such a configuration of a heart-tissue-conforming element allows additional perfusion area (i.e., cross sectional area) for blood to flow past the heart-tissue-conforming element, e.g., proximally to distally or vice versa.

In embodiments where the heart-tissue-conforming element comprises a series of circumferentially located mid-radius balloons, such mid-radius balloons may have a diameter of between 1 mm to 20 mm, such as 1 mm or 5 mm or 6 mm or 10 mm or 20 mm. In embodiments where the heart-tissue-conforming element comprises a series of circumferentially located mid-radius balloons, there may be between one to 30 or more balloons, such as one balloon or two balloons or three balloons or four balloons or five balloons or six balloons or seven balloons or eight balloons or nine balloons or ten balloons or 20 balloons or 30 balloons or more. Such balloons may all be the same diameter or may have different diameters. Additionally, such balloons may have varying diameters across their lengths, for example, such that proximal and/or distal regions of a balloon has a larger or smaller diameter than at the mid-length of the balloon.

### Balloons of Heart-Tissue-Conforming Element:

In embodiments, a balloon of the heart-tissue-conforming element, such as a mid-radius balloon or circumferentially located configuration of mid-radius balloons, as described above, may be made of any convenient biocompatible material, as such are known in the art. For example, such balloons may be made of a compliant or semi-compliant or non-compliant material, such as, in the case of a non-compliant material, a polyamide, such that the diameter of the balloon is substantially fixed (i.e., remains substantially constant) regardless of the inflation pressure applied to the balloon, or a polyimide or a combination of a thermoplastic or thermoset with a metallic or fiber braid.

Any convenient balloon may be employed as part of the heart-tissue-conforming element. Suitable balloons include, but are not limited to, standard angioplasty balloons, such as compliant and non-compliant angioplasty balloons, or, in other cases, a composite balloon that includes two distinct layers, which layers include a non-compliant layer and compliant layer. In one embodiment of a composite angioplasty balloon, a non-compliant balloon is covered with a compliant sleeve to achieve an “arrowed” pressure-stretch, e.g., as further described in pending PCT application serial no. PCT/US2020/055458; the disclosure of which is herein incorporated by reference. The compliant layer may be a rubber, silicone, polyurethane, or nitinol material or another material that can stretch up to 100-500% before failure, can withstand thousands of inflation cycles before failure, and encounters minimal, if any, plastic deformation during expansion.

In certain instances, the heart-tissue-conforming element comprises a thin sheath of material placed over its external surface, e.g., over all or a subset of balloons comprising the heart-tissue-conforming element, in order to define an exterior surface of the heart-tissue-conforming element. Such thin sheath of material may be made of any convenient biocompatible material, as such are known in the art. For example, such sheath may be made of a compliant or non-compliant polymeric material, such as polyamide or polyimide or a combination of a thermoplastic or thermoset with a metallic or fiber braid.

In embodiments, balloons, e.g., a mid-radius balloon, of the heart-tissue-conforming element may be configured such that they are each fused to each other, to a distal region of the catheter as well as to a thin material sheath such that the overall structure of the heart-tissue-conforming element is rigid and may provide a substantial radial force (e.g., to cardiovascular tissue) without collapsing inwards (e.g., collapsing axially). In such an embodiment, one or more internal areas between balloons of the heart-tissue-conforming element may also serve to allow blood to perfuse past the heart-tissue-conforming element, even while such balloons are inflated and expanded. In an exemplary case where the heart-tissue-conforming element comprises six, 6 mm diameter, non-compliant balloons positioned circumferentially around a 6 mm diameter distal region of a catheter, the space between such balloons provides approximately 10-mm² of cross-sectional area for blood to flow past the heart-tissue-conforming element, e.g., proximally to distally.

In embodiments, a surface of the balloons, e.g., mid-radius balloons, or thin sheath covering of the heart-tissue-conforming element may comprise an active agent, e.g., an external surface may be coated with an active agent, such as, for example, a drug or other medication or substance that can have a physiological effect, such as an anticoagulant or otherwise, or may comprise a hydrophilic or hydrophobic surface.

### Winged Balloons:

In embodiments configured to fracture and break-up calcium or hardened deposits on cardiovascular tissue, such as a heart valve, techniques such as, for example, pulsatile intravascular lithotripsy, may be used to generate high intensity stress waves in the calcium structures. Facilitating such treatments, in some embodiments, the heart-tissue-conforming element further comprises one or more winged balloons (or outer balloons) located on an external surface of the one or more mid-radius balloons or the outer surface of a circumferentially located configuration of mid-radius balloons. For example, an embodiment may comprise three separate winged balloons equally spaced from each other around the circumference of the one or more mid-radius balloons or the outer circumference of a circumferentially located configuration of mid-radius balloons of the heart-tissue-conforming element. Such embodiments may be configured such that during expansion of one or more mid-radius balloon, the winged balloons may be activated, for example, by using standard pulsatile intravascular lithotripsy techniques. That is, the system may be configured so that such winged balloons may be separately and independently pressurized from the one or more mid-radius balloons. In embodiments, winged balloons may be affixed at distal and proximal locations (e.g., distal and proximal to the one or more mid-radius balloon) on the catheter, or other elements of the heart-tissue-conforming element, and may be fluidically coupled such that fluid may be fed to them by small tubes that traverse internal or external to the catheter (i.e., dedicated fluidic passages of the catheter). In embodiments, the small tubes for feeding winged balloons may be formed from any physiologically suitable material and may have any convenient diameter and such may vary, for example, the diameter of such small tubes may range between 0.1 mm and 4 mm, such as 0.1 mm, 0.5 mm, 1 mm, 1.5 mm, 2 mm, 3 mm or 4 mm.

In embodiments, the heart-tissue-conforming element may be configured such that each of the winged balloons is fixed in a single location along the one or more mid-radius balloons or circumferentially located configuration of mid-radius balloons or configured such that the winged balloons are allowed to translate, e.g., circumferentially around the one or more mid-radius balloons, to find a path of least resistance to a desired region of cardiovascular tissue (e.g., to a valve commissure, where the winged balloon is configured to engage with a heart valve commissure) during expansion of the one or more mid-radius balloons and pulsation of the winged balloons. In embodiments, winged balloons may be configured and arranged on the heart-tissue-conforming element so that, upon applying pulsatile energy to the winged balloons, the winged balloons serve to orient and/or locate the heart-tissue-conforming element relative to, e.g., a heart valve by engaging, e.g., heart valve commissures. That is, upon applying pulsatile energy to the winged balloons, the winged balloons may be configured to seat themselves over, e.g., the heart valve commissures. Upon orienting the heart-tissue-conforming element as such, different pulsatile energies may be applied to heart valve commissures in contrast with heart valve leaflets, i.e., pulsatile energy transmitted by winged balloons is transferred to heart valve commissures whereas pulsatile energy transmitted to one or more mid-radius balloons or a circumferentially located configuration of mid-radius balloons is applied to heart valve leaflets. That is, a heart-tissue-conforming element may be configured such that different aspects of cardiovascular tissue can receive different forms of specific, targeted and focused pulsatile energy.

In embodiments, winged balloons are configured to pulse at any convenient rate called for by the applicable treatment and such may vary, for example, at rates between 2-50 Hz, such as 2 Hz or 5 Hz or 10 Hz or 20 Hz or 30 Hz or 40 Hz or 50 Hz, such that the pulsing of the winged balloons causes the heart-tissue-conforming element to apply a cyclical loading to cardiovascular tissue, such as a heart valve commissure. Additionally, in embodiments, the system may be configured so that a duty cycle (i.e., an on and off period) of the winged balloons may be adjusted and any convenient duty cycle may be applied. Further, in embodiments, the pressure and/or force profile of the winged balloons may be adjusted such that the pressure in a balloon oscillates between, for example, 0 to 10 atm on the trough of the cycle, and 3 to 50 atm on the peak of the cycle, such as between 0 atm on the trough of the cycle and 50 atm on the peak of the cycle or 0 atm on the trough of the cycle and 3 atm at the peak of the cycle or 3 atm at the trough side of the cycle and 50 atm at the peak of the cycle or 3 atm at the trough of the cycle and just over 3 atm at the peak of the cycle. In embodiments comprising more than one winged balloon, such balloons may be configured so that all the winged balloons are pulsed in a synchronized manner or are pulsed independently of each other, i.e., the winged balloons may be synchronously pressurized and depressurized, i.e., at the same time, or may be pressurized and depressurized independently and at different times; e.g., the winged balloons may be inflated in a sequential manner such that an intermediate shape of the heart-tissue-conforming element as the winged balloons inflate conforms to a desired shape or feature of the cardiovascular tissue that, for example, promotes alignment of the heart-tissue conforming element with the cardiovascular tissue. Other embodiments with winged balloons may be configured so that different energies may be supplied to different areas of the winged balloons balloon. For example, in some instances, it may be desirable for a greater amplitude or frequency of pulsatile energy to be applied in a central region of one or more winged balloons (e.g., a central region between proximal and distal ends of winged balloons), relative to the tips of the winged balloon (e.g., proximal or distal regions of winged balloons); e.g., pressurizing the tips of the winged balloons may be paused in order to allow for more energy to be applied to a stenotic valve as compared with a left ventricular region or aortic region. In embodiments with both winged balloons and mid-radius balloons, such balloons may be configured so that the mid-radius balloons and winged balloons are pulsed in a synchronized manner or are pulsed independently of each other, i.e., the mid-radius balloons and winged balloons may be synchronously pressurized and depressurized, i.e., at the same time, or may be pressurized and depressurized independently and at different times.

Further, in embodiments, characteristics of pulsatile energy applied by winged balloons can change over the course of using winged balloons to apply pulsatile energy to cardiovascular tissue. Changes to characteristics of pulsatile energy applied by winged balloons may be defined in advance, i.e., pre-programmed, using, for example, a controller, such as a pre-programmed controller, or the like, or can be changed dynamically, i.e., on the fly, based on any convenient parameter, such as, for example, sensor inputs, such as a pressure or volume measurement (e.g., a pressure or volume of a balloon) or based on other feedback signals or a user input or the like. For example, pulsatile energy applied by winged balloons can continuously increase or continuously decrease (i.e., the maximum or minimum or average energy applied to winged balloons can continuously increase or continuously decrease) or pause (i.e., the maximum or minimum energy applied to the balloons can remain constant, including, for example, by applying no pulsatile energy, e.g., pressure, to the balloons for a period of time, i.e., a relaxation period). In addition, in embodiments, pulsatile energy applied by winged balloons can slow down or speed up (i.e., pulsatile energy applied by winged balloons can be apply at higher or lower frequencies, or in other cases, the rate of change of a maximum or minimum or average energy applied by winged balloons can be increased or decreased).

In embodiments, the winged balloons may have any convenient shape, and such may vary based on, for example, features of the cardiovascular tissue or calcium deposits of the cardiovascular tissue. For example, in embodiments, the winged balloons may be cylindrical in shape or may be triangular in cross section such that a relatively wider side of the triangle is seated against one or more mid-radius balloons. Embodiments may be configured to facilitate dispersing pulsatile force applied to the winged balloons to a larger area of cardiovascular tissue around the one or more mid-radius balloons. In such embodiments in which a winged balloon is triangular in cross section, the pointed side of the triangular balloon may be oriented toward a valve commissure such that pulsatile energy of the heart-tissue-conforming element is particularly directed to such valve commissure. In embodiments, the winged balloons may be made of any convenient biocompatible non-compliant or semi-compliant material, i.e., the same or similar material as a mid-radius balloon.

Any convenient balloon may be employed as a winged balloon or outer balloon affixed to one or more mid-radius balloons or the exterior surface of a circumferentially located configuration of mid-radius balloons. Suitable balloons include, but are not limited to, standard angioplasty balloons, such as compliant and non-compliant angioplasty balloons. In one embodiment, a winged balloon is a composite balloon that includes two distinct layers, which layers include a non-compliant layer and compliant layer, e.g., as further described in U.S. Application No. 63/274,832, the disclosure of which is herein incorporated by reference. In one embodiment of a winged balloon comprising a composite angioplasty balloon, a non-compliant balloon is covered with a compliant sleeve to achieve an “arrowed” pressure-stretch, e.g., as further described in pending PCT application serial no. PCT/US2020/055458, the disclosure of which is herein incorporated by reference. The compliant layer may be a rubber, silicone, polyurethane, or nitinol material or another material that can stretch up to 100-500% before failure, can withstand thousands of cycles (i.e., expansion/relaxation cycles or applications of pulsatile energy) before failure, and encounters minimal, if any, plastic deformation during expansion.

In certain instances, a winged balloon may comprise a cutting or scoring element on its external surface such that the expansion and pulsation of the balloon enables cutting through or scoring or otherwise further stressing calcified or stenotic tissue. In embodiments, as described above, a distal region of the catheter where the winged balloons as well as one or more mid-radius balloons are located may be configured to provide structural support for the winged balloons to deliver a substantial radial force, i.e., force applied to the cardiovascular tissue, such that calcified cardiovascular tissue may be compressed, bent, tensioned or otherwise stressed in order to fracture internal and/or external calcium of the cardiovascular tissue. In some instances, the pulsatile stress delivered to the calcified cardiovascular tissue may generate low stress fatigue fracture of calcium in the cardiovascular tissue. In embodiments, to generate fatigue fracture, in some cases, hundreds or thousands or more pulsatile cycles may be required before low stress fracture of the calcium occurs. In such cases, perfusion mechanisms, e.g., for perfusing blood to distal vessels as described herein, may be employed to extend the treatment time before hypotension occurs or other adverse effects result from lack of perfusion past the heart-tissue-conforming element.

In other embodiments, the heart-tissue-conforming element may comprise pulsatile balloons such as winged balloons comprising elements configured to create cavitation or plasma bubbles or other forms of pulsatile or vibratory energy that cause fracturing of calcium in cardiovascular tissue. In embodiments, such elements may comprise, for example, one or more wires coupled to electrodes (e.g., one wire coupled to two electrodes) or one or more optical fibers.

In other embodiments, the heart-tissue-conforming element may comprise pulsatile balloons such as winged balloons configured to deliver an active agent, such as, for example, a drug, to a treatment site, i.e., cardiovascular tissue or tissue proximal thereto, to, for example, prevent refusion of valve commissures. In certain instances, the active agent may be coated onto the outside of a winged balloon of the heart-tissue-conforming element or other external surface of the heart-tissue-conforming element or may be delivered through fissures or pockets in a winged balloon of the heart-tissue-conforming element or other external surface of the heart-tissue-conforming element, such as, for example, a balloon wall (e.g., of a winged balloon or a mid-radius balloon or a sheath covering a plurality of mid-radius balloons). In some cases, the heart-tissue-conforming element is configured so that the active agent will migrate into cardiovascular tissue in a slow fashion over the course of treatment, or in other cases, the heart-tissue-conforming element is configured so that the active agent is dissolved after a certain number of pulses are reached during treatment.

In some embodiments, the winged balloons and/or one or more mid-radius balloons may be configured to deliver pulsatile lithotripsy to cardiovascular tissue to ensure optimal fitment of a heart valve, such as a replacement heart valve, or to reseat an already placed replacement heart valve. In certain embodiments, the winged balloons may be configured such that they can be deflated after successful treatment of cardiovascular tissue, such as a heart valve, such that the one or more mid-radius balloons may be further inflated to perform a final expansion of the cardiovascular tissue (i.e., via expansion of the one or more mid-radius balloons), such as a heart valve and/or surrounding annulus.

### Pulsatile Energy Applied to the Heart-Tissue-Conforming Element:

In embodiments in which the heart-tissue-conforming element comprises one or more balloons, e.g., a mid-radius balloon, winged balloon, etc., such balloon may be inflated and deflated in connection with applying pulsatile energy to cardiovascular tissue. In such embodiments, a balloon of a heart-tissue-conforming element may be inflated or deflated at any convenient rate, and such may vary. In general, inflation of a balloon is repeated in time, i.e., one inflation after one deflation. A configuration of inflation and deflation cycles may be chosen to facilitate blood flow, such as, for example, from a distal region of cardiovascular tissue, e.g., a distal region of a heart valve, to a proximal region of cardiovascular tissue, e.g., a proximal region of a heart valve. In such embodiments, the one or more balloons of the heart-tissue-conforming element may be inflated and deflated at any desired frequency or rate, and such may vary. In embodiments, a rate of inflation and deflation may correspond to a cardiac cycle of the cardiovascular tissue to which the heart-tissue-conforming element is applied. For example, the rate of inflation and deflation of the one or more balloons of the heart-tissue-conforming element may be once every heartbeat, or the rate may be synchronized with a clinician-adjusted heart rate such as during rapid ventricular pacing. In some instances, the catheter assembly of the system comprises at least one port of a perfusion entrance or exit zone, such as described above (i.e., configured to facilitate perfusion past the heart-tissue-conforming element), and such port may be pulsed in synchrony with the heart cycle of the cardiovascular tissue such that flow through the port is obstructed during a first segment of the heart cycle and is opened during another segment of the heart cycle. For example, when an embodiment of the system is used in connection with treating cardiovascular tissue comprising an aortic valve, at least one port of a perfusion entrance or exit zone may be obstructed during the P-S waves measured by electrocardiogram (ECG), which allows for atrial systole, isovolumic contraction of the ventricle and prevention of reverse flow from the aorta, and opened during the ST segment measured by electrocardiogram (ECG), which allows for ventricular diastole.

In some embodiments, a more complex inflation process is applied. In such embodiments, one or more balloons of the heart-tissue-conforming element are inflated and pulsate simultaneously in diastole and then collapse in systole. That is, during diastole, a first set of one or more balloons of the heart-tissue-conforming element are inflated and, simultaneously, a second set of one or more balloons of the heart-tissue-conforming element are inflated and pulsate, followed by, in systole, both the first and second sets of balloons of the heart-tissue-conforming element are collapsed. For example, the one or more mid-radius balloons may be inflated in diastole as, simultaneously, the one or more winged balloons attached to the one or more mid-radius balloons may be inflated and pulsated also in diastole, and then the one or more mid-radius balloons as well as the one or more winged balloons may be collapsed in systole. Some embodiments that utilize such an inflation process comprise more than one potential source, i.e., more than one console assemblies, at least one associated with inflating and deflating the one or more mid-radius balloons and at least one more associated with inflating, applying pulsatile energy to, and deflating the one or more winged balloons.

Heart-Tissue-Conforming Element with Radial Members:

In embodiments, the heart-tissue-conforming element comprises a plurality of radial members attached to the catheter. Such radial members may be configured and arranged on the catheter so that they can engage cardiovascular tissue by bending or flexing or folding or tensioning cardiovascular tissue to disrupt calcifications. For example, such radial members may be arranged on the catheter so that they can engage a single heart valve leaflet between the radial members so as to flex the leaflet between the radial members.

Such radial members may be positioned so that they are staggered from each other along a distal region of the catheter and may comprise eccentric, triangular or rectangular plates and/or may comprise balloons. In embodiments, the radial members are configured so that they extend radially, i.e., away from the longitudinal axis of the catheter, such that the radial members protrude outwards, radially. In embodiments, a radial member may have any convenient length, width and depth configured to sufficiently engage with cardiovascular tissue, such as a heart valve. Radial members may extend radially at a length that is greater than their cross-sectional width, meaning that when viewed in profile they take a substantially rectangular shape. For example, embodiments of radial members may have a length ranging from approximately 2 mm to 150 mm, such as, for example, 20 mm to 60 mm or 2 mm or 10 mm or 20 mm or 30 mm or 40 mm or 50 mm or 60 mm or 70 mm or 80 mm or 90 mm or 100 mm or 110 mm or 120 mm or 130 mm or 140 mm or 150 mm; a width ranging from 2 mm to 20 mm, such as, for example, 2 mm or 5 mm or 10 mm or 15 mm or 20 mm; and a radius ranging from 2 mm to 50 mm, such as, for example, 25 mm to 35 mm or 2 mm or 10 mm or 20 mm or 30 mm or 40 mm or 50 mm. In embodiments, a radial member may have any desired angle of revolution around the longitudinal axis of the catheter to which it is attached, i.e., between 0° and 360°, such as an angle of revolution between and 90°, including between 30° and 45°. By “angle of revolution,” it is meant the angle between the two radii of the sector occupied by the radial member when viewed in cross-section, along the longitudinal axis of the catheter. In embodiments where a radial member has a 360° angle of revolution, such radial member is equivalent to a lobe or a hemisphere of a yo-yo configuration of the heart-tissue-conforming element, as described herein.

In embodiments, radial members comprise radial balloons. In such embodiments, the shape of the radial balloons may be substantially cylindrical and may be conformed to a final shape during inflation by applying a constraining element to the balloon, such as, for example, a braid made from, for example, nitinol. In some cases, the shape of a radial balloon may be configured, i.e., formed into the final shape, during the balloon molding process such that, during inflation, the balloon takes the appropriate, desired shape. Radial balloons of interest may be made of any suitable physiologically compatible material such as a non-compliant material or a combination of non-compliant and compliant materials such that, during inflation, the radial balloon extends only toward specific cardiovascular tissue, e.g., toward a calcified leaflet, causing a force to be applied, e.g., bending force, to such specified cardiovascular tissue, e.g., a calcified leaflet, to fragment or otherwise disrupt embedded calcium. Any convenient balloon may be employed as a balloon of a radial member. Suitable balloons include, but are not limited to, standard angioplasty balloons, such as compliant and non-compliant angioplasty balloons. In one embodiment, a balloon of a radial member is a composite balloon that includes two distinct layers, which layers include a non-compliant layer and compliant layer, e.g., as further described in U.S. Application No. 63/274,832, the disclosure of which is herein incorporated by reference. In one embodiment of a balloon of a radial member comprising a composite angioplasty balloon, a non-compliant balloon is covered with a compliant sleeve to achieve an “arrowed” pressure-stretch, e.g., as further described in pending PCT application serial no. PCT/US2020/055458, the disclosure of which is herein incorporated by reference. The compliant layer may be a rubber, silicone, polyurethane, or nitinol material or another material that can stretch up to 100-500% before failure, can withstand thousands of cycles (i.e., expansion/relaxation cycles or applications of pulsatile energy) before failure, and encounters minimal, if any, plastic deformation during expansion.

In embodiments, balloons of radial members of the heart-tissue-conforming element may be inflated with fluid via a fluidic coupling between the interior of the balloon and a fluidic passage of the catheter. Such a fluidic coupling between the interior of the balloon and a fluidic passage of the catheter may comprise one or more porting holes configured so that the interior of the balloon is in fluidic communication with the fluidic passage. Any convenient number, size and configuration of porting holes may be applied to operably connect the fluidic passage of a catheter to a balloon of a radial member. Porting holes in embodiments may be radial holes connecting, for example, an inner wall of a fluidic passage of the catheter (i.e., the surface closest to the fluidic passage's longitudinal axis) with the external wall of the fluidic passage (i.e., the surface furthest from the longitudinal axis, for example, the surface to which the radial member is attached). Porting holes may be configured to allow the passage of fluid between the fluidic passage and a balloon of the radial member. In embodiments, a balloon of a radial member may be inflated using fluid transferred into the interior of the balloon from the fluidic passage of the catheter via the porting holes of the fluidic passage. That is, in some embodiments, pressure applied to the proximal end of the fluidic passage, propagated along the fluidic passage of the catheter, may further propagate to the balloon of the radial member via the porting holes.

The number of porting holes may vary in embodiments of the invention. Porting holes may be any convenient diameter and their diameter may vary. The arrangement of the porting holes on the fluidic passage may assume any convenient pattern. The number of porting holes, their diameters, and their arrangement on the fluidic passage of the catheter may be selected so as to ensure that the catheter retains its desired structural characteristics, notwithstanding the presence of porting holes. In some embodiments, specifically, the number of porting holes, their diameters and their arrangement on the catheter may be selected to ensure that the catheter maintains sufficient radial stiffness for supporting the action of the radial member, including expansion of the balloon of the radial member, and the application of pulsatile energy to cardiovascular tissue via the radial member. For example, some embodiments of catheters according to the present invention may comprise between 1 and 5,000 porting holes (such as 100 or 300 or 1,000 or 3,000), with diameters between 0.1 mm and 0.5 mm (such as 0.1 mm or 0.25 mm or 0.4 mm), spaced at least 0.1 mm to 5 mm apart from one another (such as 0.5 mm or 1 mm or 5 mm).

In some cases, the radial members are arranged to form a three-point flexure configuration. Radial members may comprise balloons configured so that upon pressurization of the balloon, the balloon expands so that the radial members bend or flex or fold the cardiovascular tissue. In some cases, radial members comprise balloons configured to expand and contract substantially along proximal or distal directions (i.e., as opposed to radially) so that cardiovascular tissue, such as a heart valve leaflet, present between radial members is bent or flexed or folded upon expansion of balloons. Embodiments may have one or more radial members configured so that, when pulsatile energy is received by such radial members or when such members are expanded, e.g., via inflation of fluid, such members engulf an aspect of cardiovascular tissue, e.g., a valve leaflet, to compress, bend, or tension internal calcifications of the cardiovascular tissue such that the calcifications fracture. In instances where the heart-tissue-conforming element comprises radial members, the catheter assembly may be configured to force a first radial member to translate laterally relative to a second radial member, i.e., enabling the radial members to better engage with cardiovascular tissue and to cause cardiovascular tissue engaged by the radial members to be flexed, bent folded or otherwise tensioned between radial members. In embodiments, a heart-tissue-conforming element comprising radial members may be configured to engage a single valve leaflet at a time. In instances, the catheter assembly may be configured to enable the heart-tissue-conforming element to turn and/or re-orient itself relative to the cardiovascular tissue so that the radial members are able to treat, for example, each leaflet of a heart valve sequentially.

In embodiments, radial members comprise a surface of radial members that is configured to engage or contact or apply force to (i.e., crush) cardiovascular tissue, e.g., a heart valve leaflet. Such surface of a radial member that interfaces with cardiovascular tissue may comprise the material of a radial balloon itself or may be configured to have any number of one or more elements attached thereto to facilitate disrupting calcium deposits in cardiovascular tissue, such as a nitinol cutting or scoring element, protruding features (such as a diamond-shaped or hemisphere-shaped, or the like, object on the surface to enhance stress concentrations) or a serrated scoring pattern.

### Sensors:

In certain embodiments, the catheter assembly, including, for example, the heart-tissue-conforming element and/or the catheter, may comprise one or more sensors (e.g., pressure, temperature, volume sensor or the like) configured to acquire data from one or more locations throughout the catheter assembly, i.e., as described above in connection with a controller. Some embodiments may comprise one or more sensors at one or more locations throughout the system; for example, embodiments may comprise one or more sensors in the connector of the catheter assembly, in the catheter or in the heart-tissue-conforming element, e.g., one or more balloons of the heart-tissue conforming element. In some cases, sensors are present proximal and/or distal to the heart-tissue-conforming element and are configured to measure, for example, pressure being generated by the heart (i.e., contraction of the cardiovascular tissue) or distal to the heart or a pressure gradient across the heart-tissue-conforming element.

In such embodiments, the sensors are configured to transmit to and/or receive information from a controller, such as described above, for example a controller of the console assembly. In such instances, data collected from sensors may be fused in order to evaluate behavior of the system or cardiovascular tissue that occurs at different frequencies or amplitudes of pressure applied to aspects of the heart-tissue-conforming element. The controller may be configured to read data from sensors and adjust the treatment applied to cardiovascular tissue in the form of a feedback and/or a feedforward loop. The controller may also be configured to determine a more optimal treatment profile or treatment plan (i.e., system configurations, such as, pressure, frequency and/or duty cycle configurations, for implementation by the system to address calcifications present on cardiovascular tissue). The controller may also be configured to adjust the treatment profile such that the treatment is performed according to formerly determined, optimal treatment profile. Further details regarding configuring and adjusting treatment plans are described in U.S. Application Ser. No. 63/346,704 titled “Systems and Methods Related to Catheter-Based Procedures” and filed on event date herewith (Attorney Docket No. AVSI-005PRV); the disclosure of which is incorporated herein by reference. Any convenient commercially available sensors (e.g., pressure, temperature, volume sensor or the like) may be utilized and integrated into aspects of the system as needed based on the sensor, for example, pressure and/or volume sensors may be integrated into an embodiment of a connector of the catheter assembly.

### Robotic Aspects of the System:

Another aspect of embodiments of systems relates to robotic treatment of diseased cardiovascular tissue. In one configuration, an embodiment of a system according to the present invention, including a console assembly, manifold assembly and catheter assembly are located at a treatment location, i.e., a location where the system is used to treat a subject, such as a procedure room or operating room. Connected to the treatment location is a control room comprising system controls and/or system results, e.g., data collected by sensors of the system. The control room may be operably connected to the components of the system present at the treatment location via any convenient connection, such as a wired or wireless data connection. The control room may include various displays for displaying information related to a procedure using an embodiment of a system of the invention such as for imaging or device sensors. Additionally, the control room may include a treatment controller such that an operator may be able to control various aspects of the treatment such as treatment initiation and device positioning, i.e., position of catheter assembly, including heart-tissue-conforming element with respect to cardiovascular tissue, i.e., cardiovascular tissue of a subject present in the treatment room. Other aspects may include information about a subject as well as a connection into a procedure database, i.e., configured to generate, store and/or transmit treatment plan information related to controlling aspects of the system when applying the system to cardiovascular tissue of a subject. As described above, a treatment profile or treatment plan may comprise system configurations, such as, pressure, frequency and/or duty cycle configurations, for implementation by the system to address calcifications present on cardiovascular tissue.

In some cases, the control room may be a few feet from the operating table such as behind a lead curtain or other form of shielding or may be located at a distinct geographic location, such as in another building or another city or country. One or more operators may be able to interact in the control room and may be able to communicate with the subject or other operators at the treatment location.

### Visualization:

Embodiments of the system may further comprise marker bands present on aspects of the catheter assembly. In embodiments, marker bands may be affixed to different components of the system, such as, for example, to various locations on the catheter, the heart-tissue-conforming element, one or more balloons of the heart-tissue-conforming element, entrance or exit zones of a perfusion mechanism or a guidewire threaded through the catheter. Marker bands may be used to visualize the position of the system, or components thereof, such as the heart-tissue-conforming element, when applied to a subject. Marker bands may further be used to confirm the appropriate location and alignment (e.g., commissural alignment) of aspects of the system, such as, for example, the heart-tissue-conforming element, in connection with providing treatment.

Marker bands used in embodiments may be any convenient, readily available, off the shelf marker band capable of being affixed, for example, swaged or crimped or heat bonded or welded or bonded, to components of the system. Marker bands of interest may be polymer bands laden with gold or platinum or tungsten or another material that facilitates visualization, such as visualization via fluoroscopy. Marker bands may be visualized via fluoroscopic or radioscopic visualization techniques.

Various aspects of systems of the invention being generally described above, elements of systems of the invention are now further reviewed in the context of specific embodiments.

### SPECIFIC EMBODIMENTS

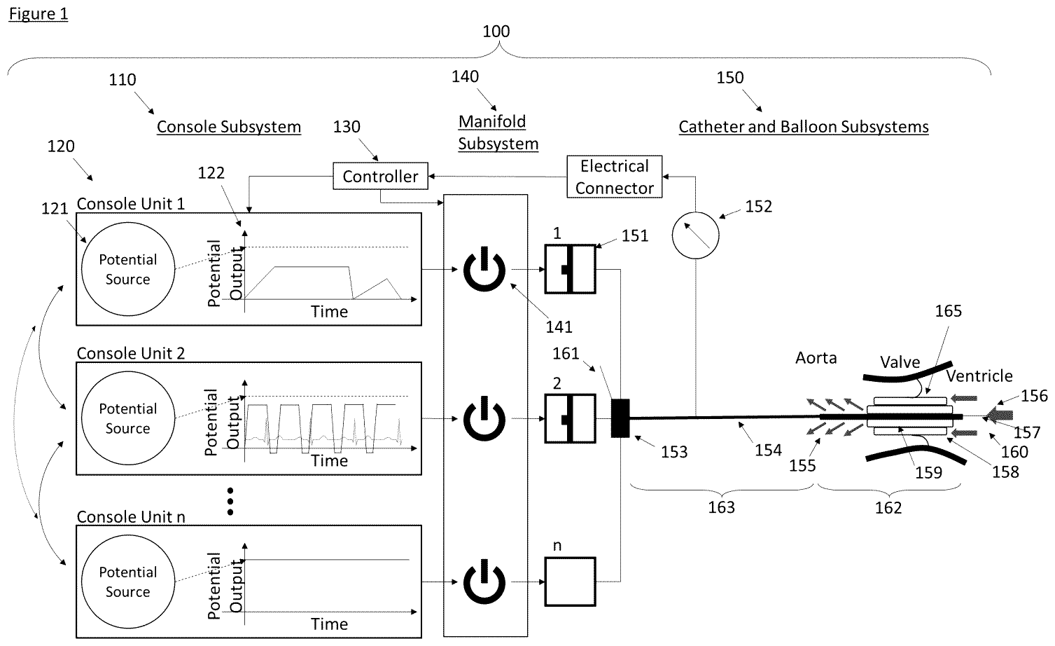

Aspects of the claimed invention are described in connection with embodiments of the system depicted in FIGS. 1 to 16 for ease of illustration only and without limiting the present invention to such embodiments.

A system in accordance with an embodiment of the invention for imparting pulsatile energy to cardiovascular tissue is schematically illustrated in FIG. 1. In some instances, the system can include a console assembly which has a potential energy source, such as a high voltage or pressure source, a regulator, e.g., for regulating the output of a pressure source, etc., and a controller, e.g., for controlling the aspects of the system. The system can also include a manifold assembly operably connected to an output of the console assembly comprising an oscillator for converting the output of the potential source into pulse energy. The system can also include a catheter assembly operably connected to an output of the manifold assembly for converting the output of the manifold assembly (i.e., first pulsatile energy), into, e.g., hydraulic oscillations or other forms of oscillatory potential energy (i.e., second pulsatile energy) and transmitting the second pulse energy via a catheter (i.e., a fluidic passage of a catheter) to a heart-tissue-conforming element. The heart-tissue-conforming element can be configured to receive oscillatory potential energy and apply pulsatile energy to cardiovascular tissue. In embodiments, the oscillatory potential energy acts to drive aspects of the heart-tissue-conforming element, such as, for example, balloon angioplasty oscillations or movement of radial members to bend or flex cardiovascular tissue. A controller can control the frequency, duty cycle and/or amplitude of the outputted energy from the potential source of the console assembly as well as the oscillator of the manifold assembly. A connector of the catheter assembly can convert energy outputted by the manifold assembly into, e.g., hydraulic oscillations, that thereby generate oscillations in aspects of a heart-tissue-conforming element. Variations of this system are provided in each of the embodiments below. The following embodiments are not meant to be an exhaustive list but are meant to provide examples of various configurations of the overall system.

FIG. 1 depicts a schematic view of an exemplary embodiment of a system 100 for imparting pulsatile energy to cardiovascular tissue according to the present invention. In some embodiments as illustrated schematically in FIG. 1, the system 100 includes three assemblies: a console assembly (i.e., console subsystem) 110, which may comprise one or more console units 120; a manifold assembly (i.e., manifold subsystem) 140, which may comprise one or more subunits including an oscillator 141; and catheter assembly (i.e., catheter and balloon subsystems) 150, which may comprise one or more connectors 151, as well as catheter 154 with a fluidic passage (not shown) and heart-tissue-conforming element 165. Each connector 151 of catheter assembly 150 is connected to an oscillator 141 of manifold assembly 140 and one or more connector-to-catheter transition hubs 153 for transmitting pulsatile energy (i.e., second pulse energy) to heart-tissue-conforming element 165 via catheter 154.

Console assembly 110 may comprise one or more console units 120. When the console assembly comprises a plurality of console units, such console units may be combined into a single physical component (i.e., housing) or separated into a plurality of housings, e.g., one housing per each console unit. In each case, console units are configured to operated independently of each other, i.e., are capable of being controlled independently, whether the console units are present in a single housing or multiple housings. Console unit 120 includes a potential source 121 for generating energy for transmission to manifold assembly 140, a potential regulator (not shown) and controller 130. The output from potential source 121 may include regulated 122 or un-regulated potential energy, such as that from high-pressure fluid or voltage. A potential regulator may be used to modify potential energy output from potential source 121 into a form that can be transmitted and further manipulated by manifold assembly 140, i.e., such that oscillator 141 can generate pulse energy from energy transmitted from potential source 121. Multiple console units 120 (i.e., console units numbered 1 through n) can be included in console assembly 110 and can operate substantially in parallel (i.e., independently) to generate multiple potential outputs 122 for transmission to multiple oscillators 141. In some instances, multiple console units 120 can be configured to generate multiple potential outputs 122 in cases where treatment of cardiovascular tissue comprises applying different configurations of pulsatile energy to cardiovascular tissue, for example, where the heart-tissue-conforming element comprises a plurality of balloons configured to be inflated independently potentially at different frequencies, duty cycles and/or amplitudes. In such instances, different potential outputs 122 may separately be applied to cardiovascular tissue, e.g., via different balloons or other aspects of the heart-tissue-conforming element or via different times (i.e., where one potential output is operably connected to a balloon at a first time and subsequently another potential output is operably connected to the balloon at a second time). In other instances, potential output 122 from multiple console units 120 may be combined. In still other instances, multiple console units 120 can be configured to generate multiple potential outputs 122 including different forms of potential energy (e.g., high pressure or voltage) in cases where treatment of cardiovascular tissue requires application of different forms of energy.

Console assembly 110 further comprises controller 130 configured to receive input from at least one of console assembly 110, manifold assembly 140 and catheter assembly 150. In FIG. 1, controller 130 is shown receiving input from catheter assembly 150, i.e., sensor 152 of catheter assembly 150. Sensor 152 can comprise any sensor configured to sense any relevant characteristic of catheter assembly 150 capable of detection. For example, sensor 152 may comprise a pressure transducer configured to measure a pressure within catheter assembly 150, such as, for example, a pressure of a fluid channel of catheter 154 or a pressure of an aspect of heart-tissue-conforming element 165 such as an angioplasty balloon; alternatively, sensor 152 may comprise a volume sensor, configured to measure a volume of fluid present in, for example, a balloon. In instances, controller 130 can be configured to receive input from a plurality of sensors, including sensors configured to measure any relevant aspect of system 100 or the environment in which system 100 is applied (e.g., pressure sensor, temperature sensor, volume sensor or the like) and may be configured to capture data from any location throughout system 100, including, for example, one or more locations of system 100, e.g., locations on heart-tissue-conforming element 165, catheter 154, connector-to-catheter transition hubs 153, connector 151, oscillator 141 or console unit 120. In general, in embodiments, sensors can be configured at any desired location of the system to gather any desired information regarding use of the system, e.g., in connection with a treatment procedure. In other instances, controller 130 can be configured to receive input from user inputs such as buttons or switches for specifying treatment options (e.g., system pressures, frequencies, duty cycles, etc.). In FIG. 1, controller 130 receives input from pressure transducer 152 of catheter assembly 150 and, based at least in part on such input, generates a control signal for controlling aspects of console assembly 110, such as, for example, a magnitude of potential output 122, i.e., via an active regulator (not shown) used to adjust a magnitude of potential output 122 (e.g., an output pressure).

In embodiments as illustrated schematically in FIG. 1, an output of console assembly 110 is operably connected to manifold assembly 140, such that energy transmitted (i.e., regulated potential output 122) from potential source 121 of console unit 120 is transmitted to oscillator 141 of manifold assembly 140. Oscillator 141 is configured to generate pulsatile or static energy from energy transmitted from potential source 121 (i.e., regulated potential output 122). In instances, oscillator 141 may include a solenoid valve (not shown) configured to either allow or interrupt transmission of energy to catheter assembly 150. In other instances, oscillator 141 may include any applicable electrical, e.g., electrical solenoid, optical or mechanical switch, as such are known in the art. As described further below, the behavior of oscillator 141 may be controlled by controller 130 based on any desired feedback, such as, for example, feedback from system 100 or external signals, such as, for example, inputs from an operator.

In FIG. 1, controller 130 is shown connected to manifold assembly 140. In instances, oscillator 141 may be configured so that an oscillation frequency and/or duty cycle may be controlled by controller 130, e.g., such that controller 130 controls a position or other aspect of the behavior of a solenoid of oscillator 141.

In embodiments as illustrated schematically in FIG. 1, an output of oscillator 141 of manifold assembly 140 is operably connected to catheter assembly 150. In particular, an output of oscillator 141 is connected to an input of connector 151. Connector 151 is configured to transduce potential energy such as, for example, pneumatic pressure (i.e., a first pulse energy), generated by oscillator 141, to a second potential energy such as, for example, hydraulic pressure (i.e., a second pulse energy). System 100 shown in FIG. 1 includes multiple connectors 151 (connectors 1 through n), one connector 151 corresponding to each oscillator 141. Outputs of connectors 151 are operably connected to connector-to-catheter transition hub 153 configured to enable potential energy output of connectors 151 (i.e., second pulse energy) to be input into catheter 154, e.g., one or more fluidic channels (not shown) of catheter 154. In some cases, catheter assembly 150 comprises more than one catheter 154 or catheter 154 comprises more than one fluidic channel internal or external to catheter 154, i.e., such that different aspects of heart-tissue-conforming element 159 may be independently operated (e.g., such that different angioplasty balloons or radial members comprising heart-tissue-conforming element 159 may be independently pressurized or inflated and deflated).

In catheter assembly 150 of system 100, catheter 154 includes a channel dedicated for a guidewire so that catheter 154 and heart-tissue-conforming element 159 can be navigated to a cardiovascular tissue treatment site via a standard over-the-wire guidewire approach. Connector-to-catheter transition hub 153 is configured to include guidewire exit port 161 for a proximal region of a guidewire threaded through a guidewire channel of catheter 154. Guidewire exit port 161 is opposite guidewire entrance port 157 present at a relatively distal region of heart-tissue-conforming element 165. For illustrative purposes only, in order to highlight the location of guidewire exit port 161 relative to other components of system 100, guidewire exit port 161 is depicted as oriented away from the longitudinal axis of catheter 154. In embodiments, guidewire exit port 161 is oriented in a manner that is parallel to the longitudinal axis of catheter 154 (and therefore parallel to the longitudinal axis of the guidewire channel within catheter 154), in order to avoid any unnecessary bends in a guidewire present in system 100. As described above system 100 may be configured with respect to the guidewire so that system 100 is an over-the-wire, rapid exchange, monorail or the like system.

In embodiments as illustrated schematically in FIG. 1, heart-tissue-conforming element 165 is present at a distal region of catheter 154. In system 100, heart-tissue-conforming element 165 is configured to conform to cardiovascular tissue comprising an aortic heart valve, such that heart-tissue-conforming element 165 spans the leaflets of the aortic valve with a distal region of heart-tissue-conforming element 165 present on the ventricle side of the aortic valve and a proximal region present on the aortic side of the aortic valve. Heart-tissue-conforming element 165 includes a plurality of angioplasty balloons and other features (e.g., locating elements or nubs or the like) configured to engage aspects of the aortic heart valve. The angioplasty balloons of heart-tissue-conforming element 165 include one or more outer commissure balloons (e.g., winged balloons) 158 and one or more inner valvuloplasty balloons (e.g., mid-radius balloons) 159. Angioplasty balloons 158,159 of heart-tissue-conforming element 165 may be independently operably (i.e., capable of pressurization and depressurization independent of whether other balloons are pressurized or depressurized).

Outer commissure balloon 158 is present at a relatively outer radial distance from the longitudinal axis of heart-tissue-conforming element 165 sufficient so that outer commissure balloon 158 can engage with (i.e., apply pulsatile energy to) an aortic valve commissure, e.g., to disrupt calcium formations in an aortic valve commissure that inhibits proper valve function. Outer commissure balloon 158 may comprise non-compliant/compliant material, as described above. Heart-tissue-conforming element 165 may comprise multiple outer commissure balloons 158 configured so that outer commissure balloons 158 engage each valve commissure of the aortic valve, for example, comprising three outer commissure balloons 158 to engage each aortic valve commissure.

Outer commissure balloon (e.g., winged balloon) 158 may be configured to be independently operable, i.e., independently pressurized and depressurized, from a second outer commissure balloon and from inner valvuloplasty balloon 159, by, for example, being pressurized by hydraulic pressure transmitted via separate fluidic chambers of catheter 134. Alternatively, outer commissure balloons 158 may be configured so that they are pressurized and depressurized simultaneously, by, for example, being pressurized by hydraulic pressure transmitted via a single fluidic channel of catheter 134 that feeds multiple outer commissure balloons 158.

Inner valvuloplasty balloon (e.g., mid-radius balloon) 159 is present at a relatively inner radial distance from the longitudinal axis of heart-tissue-conforming element 165 sufficient so that inner valvuloplasty balloon 159 can engage with (i.e., apply pulsatile energy to) an aortic valve leaflet, e.g., to disrupt calcium formations in an aortic valve leaflet that inhibits proper valve function. Heart-tissue-conforming element 165 may comprise multiple inner valvuloplasty balloons 159 configured so that inner valvuloplasty balloons 159 engage each valve leaflet of the aortic valve. For example, heart-tissue-conforming element 165 may comprise three inner valvuloplasty balloons 159 to engage each aortic valve leaflet or a single inner valvuloplasty balloon 159 configured so that different surfaces of the single balloon engage each aortic valve leaflet.

Inner valvuloplasty balloon 159 may be configured to be independently operable, i.e., independently pressurized and depressurized, from a second inner valvuloplasty balloon and from outer commissure balloon 158, by, for example, being pressurized by hydraulic pressure transmitted via a separate fluidic channel of catheter 134. Alternatively, inner valvuloplasty balloons 158 may be configured so that they are pressurized and depressurized all together simultaneously, by, for example, being pressurized by hydraulic pressure transmitted via a single fluidic channel of catheter 134 configured to feed multiple balloons.

In embodiments as illustrated schematically in FIG. 1, catheter 154 and heart-tissue-conforming element 165 are configured to allow fluid, e.g., blood, to perfuse past heart-tissue-conforming element 165 even while applying pulsatile energy to cardiovascular tissue at a treatment site. For example, in system 100, catheter 154 and heart-tissue-conforming element 165 are configured to enable blood to be displaced, i.e., perfuse, from perfusion entrance zone 156 to perfusion exit zone 155 via one or more passageways (not shown), e.g., fluidically connecting perfusion entrance zone 156 with perfusion exit zone 155, permitting blood received from a distal region (i.e., ventricular region) to exit at a proximal region (i.e., aortic region) of heart-tissue-conforming element 165. Perfusion entrance zone 156 and perfusion exit zone 155 comprise perforations or ports (not shown) in catheter 154 or heart-tissue-conforming element 165 through which blood can flow. Such a configuration enables blood flow past heart-tissue-conforming element 165 enabling extended treatment times. Such a perfusion mechanism may further comprise a structure (e.g., valve or oscillating balloon mechanism) in catheter 154 or heart-tissue-conforming element 165 permitting fluid to flow in only one direction. Catheter 154 comprises proximal catheter section 163 and distal catheter perfusion section 162. Distal catheter perfusion section 162 comprises perfusion entrance zone 156, perfusion exit zone 155 as well as one or more passageways (not shown), e.g., fluidically connecting perfusion entrance zone 156 with perfusion exit zone 155. Proximal catheter section 163 and distal catheter perfusion section 162 may be connected by a connector (not shown).

Any convenient perfusion mechanism may be employed in the catheter 154 and heart-tissue-conforming element 165 to induce the flow of fluid, e.g., blood, through a perfusion zone, i.e., from perfusion entrance zone 156 to perfusion exit zone 155, past heart-tissue-conforming element 165, i.e., displaced from a distal region to a proximal region of heart-tissue-conforming element 165 such that heart-tissue-conforming element 165 does not completely obstruct the flow of fluid, e.g., blood, when present in, e.g., a vessel. As described above, a passive perfusion mechanism may be employed to induce fluid to flow from perfusion entrance zone 156 to perfusion exit zone 155 based on an existing pressure gradient, i.e., an existing pressure differential caused by, e.g., ventricle contraction, or an active perfusion mechanism comprising, for example, a syringe, such as a barrel syringe, configured to displace fluid through a perfusion zone from perfusion entrance zone 156 to perfusion exit zone 155 and past heart-tissue-conforming element 165.

In instances, aspects of system 100, such as, for example, console assembly 110 (e.g., controller 130), manifold assembly 140 (e.g., oscillator 141) as well as aspects of catheter assembly 150 (e.g., connectors 151 and connector-to-catheter transition hubs 153) may be configured to be reusable. In instances, aspects of system 100, such as, for example, catheter 154 or heart-tissue-conforming element 159 may be configured to be used a single time (e.g., such that it is disposable). The terms “reusable” and “disposable” as employed here and elsewhere throughout the description are used for convenience in describing an embodiment of the invention illustrated in FIG. 1. However, the invention is not so limited. As such, any part of the system may be configured for one time use or for use multiple times, as desired.

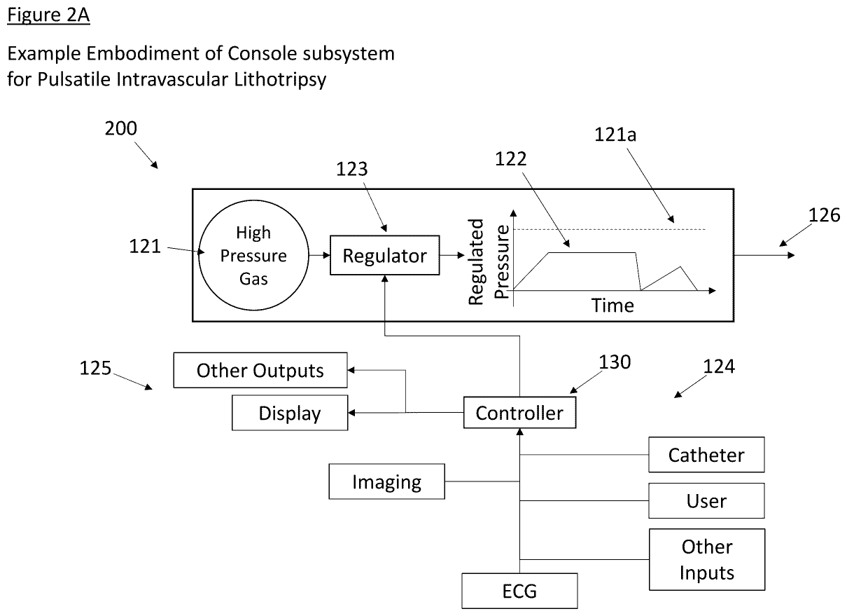

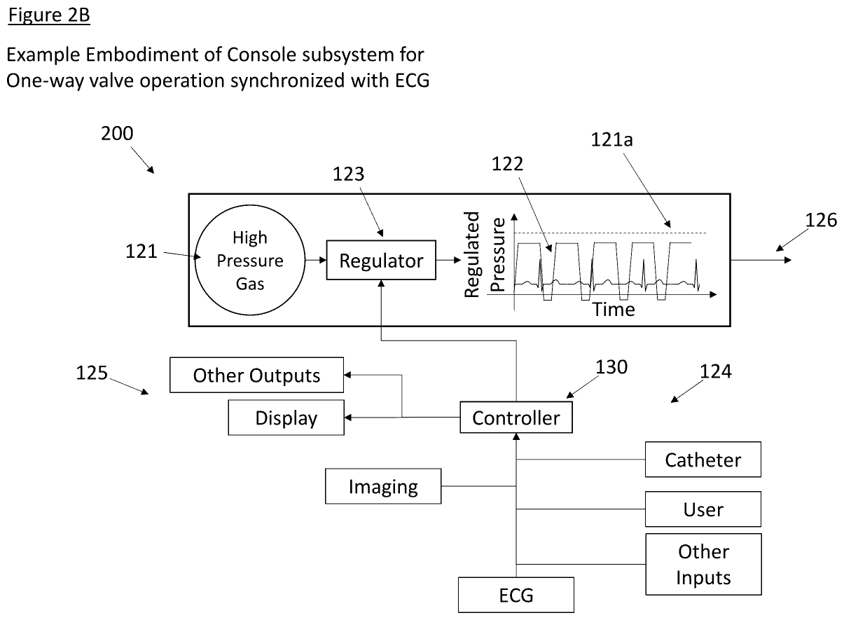

FIGS. 2A and 2B provide schematic views of a console assembly 200 according to an embodiment of the invention, e.g., that may be used in a system, such as system 100 schematically illustrated in FIG. 1. Console assembly 200 is configured to deliver regulated, high-pressure gas and controlled by a number of inputs. FIG. 2A provides a schematic view of console assembly 200 configured to provide treatment comprising, for example, pulsatile intravascular lithotripsy, and FIG. 2B provides a schematic view of console assembly 200 configured to provide treatment comprising, for example, a one-way valve operation synchronized with electrocardiogram (ECG). Console assembly 200 includes a potential source that is high pressure gas source 121. Potential energy output of high pressure gas source 121 is fed into regulator 123. Regulator 123 is configured to modify potential energy output 121a (dotted line) (i.e., a constant high pressure) of high pressure gas source 121 to regulated pressure output 122.

In FIG. 2A, a characteristic treatment profile for a lithotripsy procedure according to an embodiment of the invention is depicted by showing regulated output pressure 122 over time. As a function of time on the x-axis, regulated pressure 122 is initially slowly ramped up then held constant for a period of time. When a fracture or balloon volume change is detected, pressure is decreased before being slowly increased again, i.e., slowly re-ramped, for a final treatment segment.

In FIG. 2B, a treatment profile in which regulated output pressure 122 for applying pressure pulses the mid-radius balloon layer is timed with results of an electrocardiogram (ECG). Such a treatment profile enables system 200 to synchronize cycling regulated output pressure 122 with features of a cardiac cycle. In some embodiments, such a configuration requires vacuum to be applied (i.e., regulated output pressure 122 is depicted as periodically taking a negative pressure on the plot of regulated output pressure 122 over time) to facilitate rapid evacuation of a balloon of the heart-tissue-conforming element. Such vacuum may be applied in any convenient manner, as such are known in the art, including, for example, including a switch or solenoid or the like as part of regulator 123. Such vacuum pressure facilitates deflating and evacuating the balloon, i.e., in a rapid manner, allowing fluid, e.g., blood, to perfuse past the deflated balloon. In some embodiments, a composite compliant/non-compliant balloon material is used to ensure sufficiently rapid balloon evacuation. (In another embodiment of a treatment profile in which pressure pulses are applied to the mid-radius balloon layer in a manner that is timed with results of an electrocardiogram (ECG), regulated output pressure 122 is held constant and, instead of regulator 123, an oscillator of the manifold assembly is used to cycle balloon pressure based on results of an ECG. In such embodiments, regulated output pressure 122 (i.e., input pressure to the balloon) would cause the balloon to inflate, and an outlet of the oscillator could be attached to vacuum or to atmosphere to facilitate rapid depressurization and evacuation of the balloon.)

In FIGS. 2A and 2B, two console units 200, each with a high pressure gas source 121, are depicted in connection with the two different treatment profiles described above. In other embodiments, a single console unit 200, with one or more high pressure gas source 121, may be connected to a plurality of manifold assemblies, where each oscillator of the manifold assemblies is configured to generate distinct treatment profiles, such as the two treatment profiles described above in connection with FIGS. 2A and 2B.

High pressure gas potential energy output 121a is fed into regulator 123. Output of regulator 123, i.e., regulated pressure output 122, is transmitted to an input of a manifold assembly (not shown) via output port 126 such that regulated output pressure 122 is transmitted to the oscillator, which turns on and off to transmit pulsatile energy (with maximum pulse pressure equal to or less than regulated output pressure 122). Regulator 123 is an active regulator configured to be controlled by signals generated by controller 130. Control of regulator 123 by controller 130 comprises manipulating output pressure levels of regulator output 122, e.g., increasing or decreasing regulated pressure at specified times.

Controller 130 is an electronic component configured to receive inputs 124 including data inputs from one or more of a catheter assembly, user inputs, electrocardiogram inputs, imaging inputs, electrocardiogram inputs and other inputs, e.g., input from a manifold assembly. Inputs 124 comprising data received from a catheter assembly may include measurements of pressure applied to a heart-tissue-conforming element or volume measurements of aspects of, e.g., balloons comprising, a heart-tissue-conforming element. Inputs 124 comprising imaging data may include any relevant data capable of being imaged, such as, for example, fluoroscopy data related to a position of a heart-tissue-conforming element relative to cardiovascular tissue or a function of a heart valve or a degree of calcification of cardiovascular tissue. Such fluoroscopy data that comprises controller inputs 124 may be obtained using, for example, radiopaque markers.

Based on inputs 124 such as described above as well as a treatment plan or treatment mode, e.g., a pulsatile intravascular lithotripsy treatment mode or a one-way valve operation synchronized with electrocardiogram (ECG) mode, controller 130 generates signals comprising controller outputs 125. Controller outputs 125 include regulator control signal as well as display data, e.g., to control a user display for providing information about an ongoing treatment such as pressure measurements at various aspects of the system to a user, as well as other outputs.

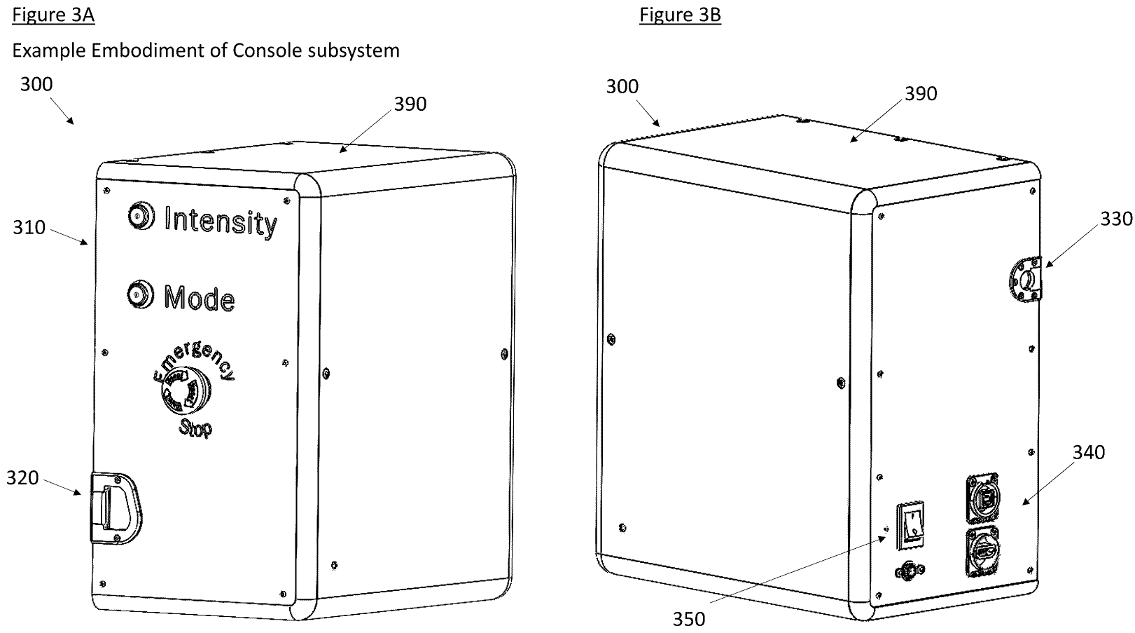

FIGS. 3A and 3B depict two views of an exemplary embodiment of a console assembly 300 configured to generate the required power and control for treatment of cardiovascular tissue using a system in accordance with embodiments of the invention. FIG. 3A provides a first perspective view of console assembly 300, and FIG. 3B provides a second perspective view of console assembly 300. Console assembly 300 includes console inputs 310 for input to a controller (not shown) for adjusting behavior of the system. Inputs 310 include buttons for selecting a treatment intensity and mode as well as an emergency shut-off button. Console assembly 300 includes console input/output ports 340, which in some cases may comprise an HDMI port or a USB port for transmitting data to and or from console assembly 300. Console assembly 300 includes on-off switch 350 for enabling and disabling the console assembly 300. Console assembly 300 also includes output port 320 for transmitting potential energy comprising a connector, e.g., a pneumatic connector or a hydraulic connector or an electrical connector or an optical connector, for connection to a manifold assembly. An external potential source, such as a connection to high pressure gas, for example, may be connected to console assembly via connector 330. Console assembly 300 also includes a console encasement 390 configured to form a housing e.g., configured to protect the console assembly components e.g., during an accidental fall or during packaging. Console encasement 390, when present, may be fabricated from a suitably rigid material, e.g., polymeric material, and may be transparent or opaque, as desired.

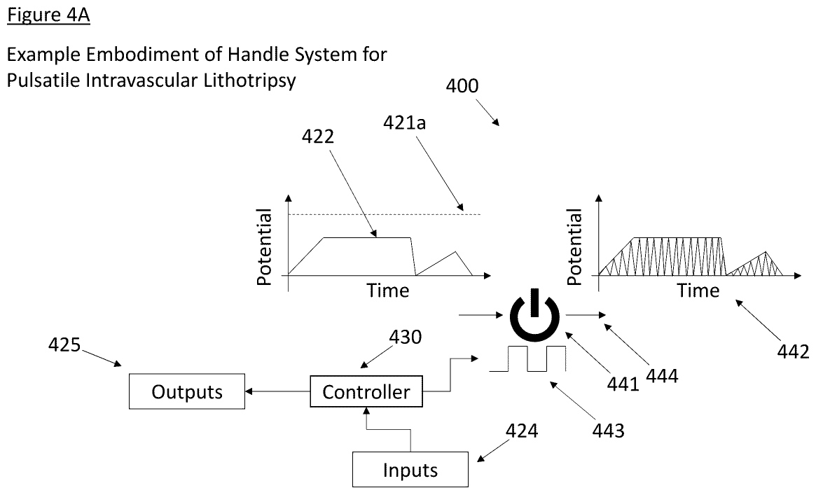

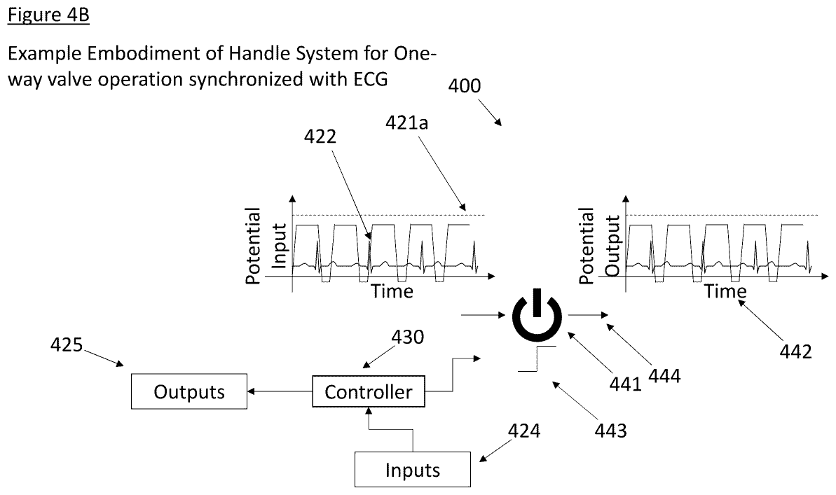

FIGS. 4A and 4B provide schematic views of a manifold assembly 400 according to an embodiment of the invention, e.g., that may be used in a system, such as system 100 schematically illustrated in FIG. 1. Manifold assembly 400 employs an oscillator 441 to convert regulated potential energy 422 received from potential source of the console assembly (not shown) to treatment energy 442 for use by the catheter assembly (not shown). Depicted in FIG. 4A is an application of manifold assembly 400 in connection with providing pulsatile intravascular lithotripsy, seen in potential output 442 where pressure changes over time correspond with a pulsatile intravascular lithotripsy treatment. The sawtooth-like shapes of potential output 442 represent oscillator output and pulsatile potential applied to cardiovascular tissue, e.g., via a balloon of the heart-tissue-conforming element. The maximum potential output 442 of oscillator corresponds to the solid line seen above potential output 442 (e.g., regulated output pressure 122 of FIGS. 2A and 2B). High pressure gas potential energy output 421a is depicted in the plot for reference. Depicted in FIG. 4B is an application of manifold assembly 400 in connection with providing a one-way valve operation synchronized with results of an electrocardiogram (ECG), seen in potential output 442 where pressure changes over time correspond with ECG results. Manifold assembly 400 includes receiving energy transmitted from a potential source (not shown) via potential source output 422. Such energy is exposed to oscillator 441, which is controlled via oscillatory control signal 443, i.e., for adjusting frequency, duration and/or duty cycle of oscillator 441, including, for example, turning off oscillator 441. FIG. 4A depicts an oscillatory control signal 443 configured to cause the oscillator to turn on and off, whereas FIG. 4B depicts an oscillatory control signal 443 configured to remain constantly on, i.e., open, such that the regulator varies potential source output 422 (i.e., regulated output pressure) based on results of an ECG, illustrating a difference in applying manifold assembly 400 in connection with pulsatile intravascular lithotripsy versus providing a one-way valve operation synchronized with results of an ECG. (In another embodiment of a manifold assembly 400 for providing a one-way valve operation synchronized with results of an ECG, potential source output 422 (i.e., regulated output pressure) is a constant pressure, and oscillator 441 is cycled to vary potential output 442 in a manner that is synchronized with results of an ECG). In some cases, synchronizing potential output with results of an ECG comprises controlling the potential applied to one or more balloons or other aspects of the heart-tissue-conforming element to collapse (i.e., collapse one or more balloons of the heart-tissue-conforming element) just before or just after the QRS spike of the ECG and then apply pressure in order to inflate one or more balloons or other aspects of the heart-tissue-conforming element in the diastolic phase with the collapse of one or more balloons or other aspects of the heart-tissue-conforming element starting before the P wave.

In any case, resulting pulse energy 442 generated by oscillator 441 of manifold assembly 400 is transmitted to catheter assembly (not shown) via oscillator output connector 444. Oscillatory control signal 443 is generated by controller 430 configured to receive inputs 424 and, based at least in part on inputs 424, generate outputs 425, including oscillatory control signal 443. In FIG. 4A, controller 430 is configured to receive input signals 424 corresponding to at least a pressure and/or volume of a connector of the catheter assembly (not shown) and/or a heart-tissue-conforming element of the catheter assembly (not shown), i.e., input signals internal to the system. In FIG. 4B, controller 430 is configured to receive input signals 424 corresponding to a pressure and/or volume of a connector of the catheter assembly (not shown) and/or a heart-tissue-conforming element of the catheter assembly (not shown), i.e., input signals that are internal to the system, as well as an input signal corresponding to results of an electrocardiogram (ECG), i.e., input signals that are external to the system. Controller 430 may be included as part of console assembly (not shown) (i.e., controller 130 of FIG. 1 or FIGS. 2A and 2B) or, in some cases, as part of manifold assembly 400.

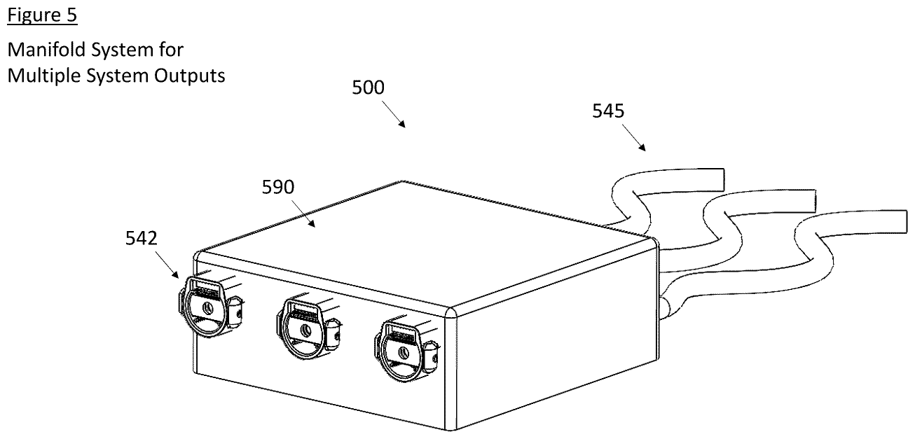

FIG. 5 provides a perspective view of manifold assembly 500 of a system in accordance with embodiments of the invention, such as manifold assembly 400 of FIGS. 4A and 4B. Manifold assembly 500 includes multiple inlet sources 545 comprising cables, such as, for example, optical or electrical cables (configured to transmit power or signals, such as data or control signals) or the like, or tubes, such as, for example, pneumatic or hydraulic tubes or the like, for receiving potential input into manifold assembly 500 as well as potential outputs 542 for transmitting energy out of manifold assembly 500. Manifold assembly 500 comprises multiple oscillators (not shown), one for each pair of inlet source 545 and potential output 542, for converting energy transmitted from the potential source into pulse energy.

Manifold assembly 500 also includes a manifold encasement 590 configured to form a housing e.g., configured to protect the manifold assembly components e.g., during an accidental fall or during packaging. The manifold encasement 590, if present, may be fabricated from a suitably rigid material, e.g., polymeric material, and may be transparent or opaque, as desired. In some cases, manifold assemblies can include controller connection points and a user feedback and/or control area (not shown).

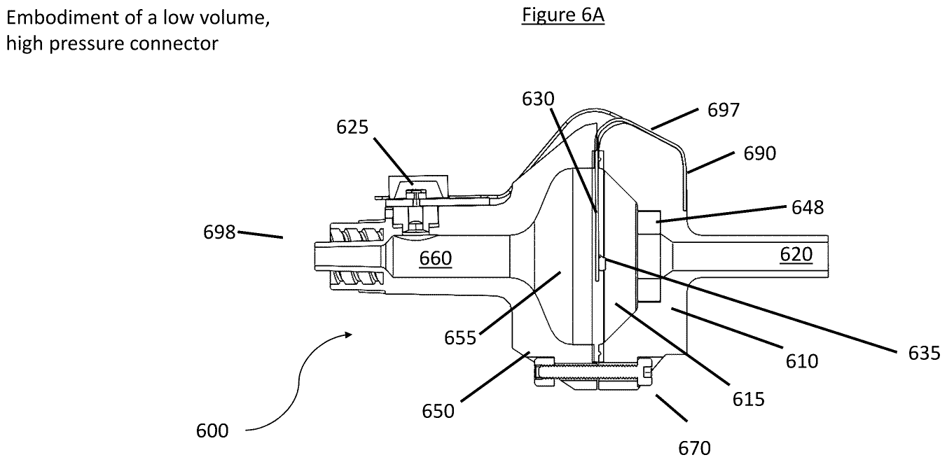

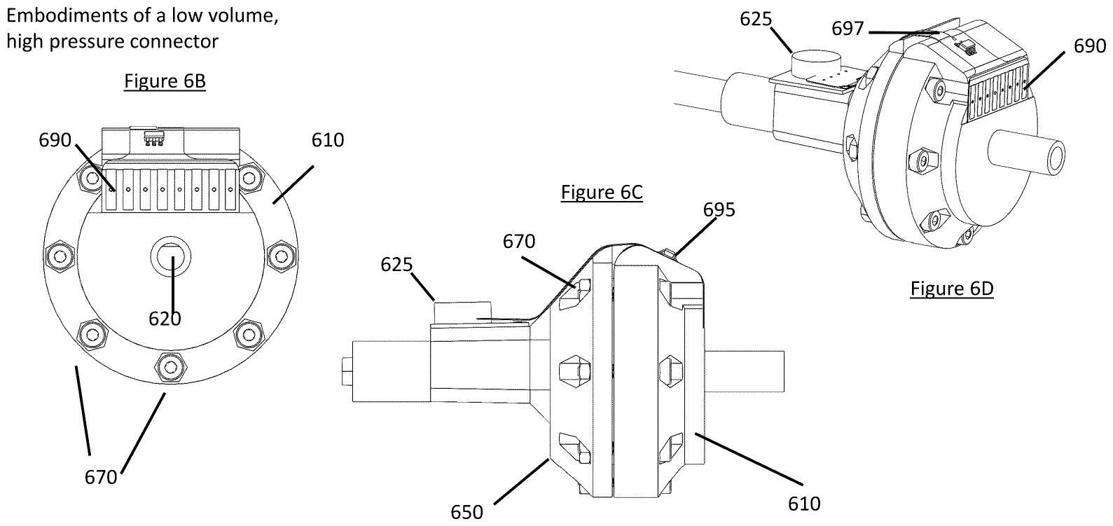

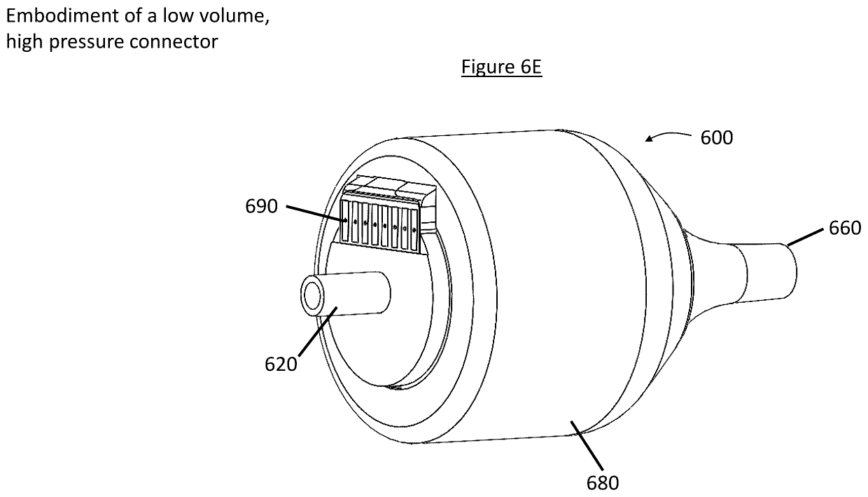

FIGS. 6A to 6E depict multiple views of an exemplary embodiment of a connector 600 configured to deliver a low volume, high-frequency, and high-pressure pulse in accordance with embodiments of the invention. FIG. 6A provides a cutaway side illustration of connector 600. Connector 600 includes proximal flange 610 and distal flange 650 separated by membrane 630. Proximal flange 610 defines proximal chamber 615 which is accessed by proximal port 620. Distal flange 650 defines distal chamber 655 which is accessed by distal port 660. Pressure transducer 625 is operably coupled to distal port 660 and distal chamber 655. The proximal and distal flanges 610,650 are held together by screws, as illustrated by screw 670. Alternatively, the flanges can be fixed via any other appropriate assembly method such as an adhesive, weld, or other means. In other instances, the flanges can be fabricated as a single component via a multi-stage injection molding or over-molding process around the flexible membrane and electronics. Also shown is Hall sensor 635, permanent magnet 648, electrical connector 690 and flexible printed circuit board 697. The threaded portion 698 at the distal end of the distal port serves as the interface between the proximal flexible tube and the distal flange.

FIG. 6B provides an end view of proximal flange 610 of connector 600. As seen in FIG. 6B, screws 670 are positioned circumferentially around the flange to provide connection to the distal flange (not shown). Also shown is proximal port 620 and electrical connector 690, which provides for an operable electrical connection to the console assembly (not shown) (i.e., to a controller)

FIG. 6C shows an outer side view of connector 600, showing proximal and distal flanges 610,650 joined together by screws 670. Also shown is memory 695, which is electrically coupled to flexible printed circuit board 697, which is electrically coupled to electrical connector 690. Similarly, pressure sensor 625 is electrically coupled to flexible printed circuit board 697. FIG. 6D shows a perspective view of connector 600.

FIG. 6E provides a view of connector 600 with over-mold 680 which is fabricated from a rigid, opaque material that serves to protect the various components, e.g., circuitry, sensors, etc., of the connector. The connector may have diameter and width variations to accommodate different types of heart-tissue-conforming elements while still being able to be connected to a common manifold assembly. For example, for peripheral or coronary heart-tissue-conforming elements, the connector may have dimensions to accommodate a volume change between 1-20 mL. For larger heart-tissue-conforming elements such as those comprising valvuloplasty balloons, the connector can be enlarged to accommodate a volume change up to 50 mL-100 mL.

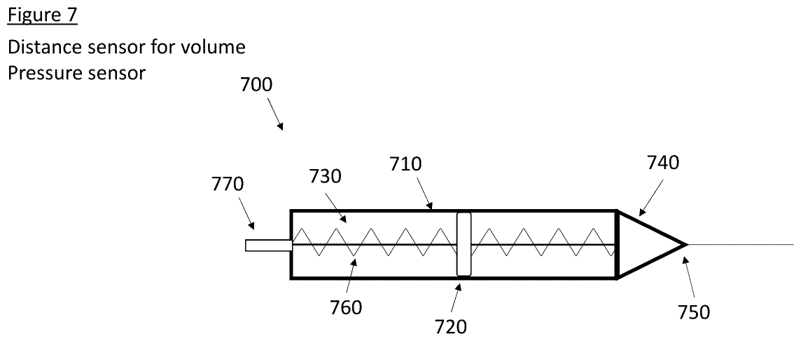

FIG. 7 provides a schematic of an alternative connector 700 configured to deliver a high-volume, low-frequency and low-pressure pulse, as described above. Connector 700 comprises barrel syringe 710 with plunger (i.e., piston) 720 separating pneumatic chamber 730 from fluid chamber 740. Pneumatic chamber 730 comprises pneumatic input port 770, configured to receive energy (i.e., a first pulse energy) from the manifold assembly (not shown), e.g., in the form of pneumatic pressure, and transmit such energy to pneumatic chamber 730. Plunger 720 is configured to translate in response to pressure applied to pneumatic chamber 730, in turn transmitting energy to fluid chamber 740. Fluid chamber 740 comprises fluid output port 750 operably connected to a catheter (not shown) and configured transmit energy to catheter (i.e., a fluidic chamber thereof) in response to movement of plunger 720 compressing fluid chamber 740. Connector 700 further comprises biasing spring 760 configured to urge plunger 720 to return to a starting position when plunger 720 is displaced, as described above.

In instances where a connector comprises an internal fluid, the connector may be configured to receive fluid through a fluidly coupled priming port (not shown). For example, such a fluid port may be connected to fluid chamber 740 in connector 700 of FIG. 7. Through such a port, a fluid such as radiopaque contrast, saline, CO₂ or the like may be injected to prime the fluid chamber. Additionally, a vacuum may be applied to such a port so that the connector as well as the system as a whole can be, for example, de-gassed prior to treatment. The priming port may be closed and sealed so that no fluid may exit the port during treatment.

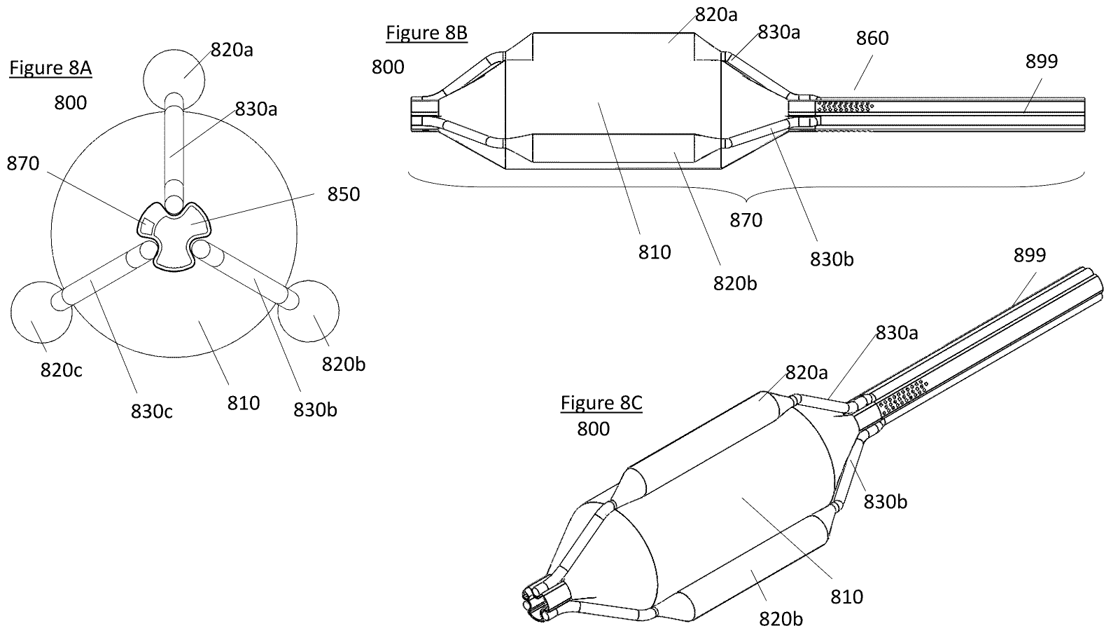

FIGS. 8A to 8C depict multiple views of an exemplary embodiment of a heart-tissue-conforming element 800 according to an embodiment of the invention, e.g., that may be used in a system, such as system 100 schematically illustrated in FIG. 1. FIG. 8A shows a front view of heart-tissue-conforming element 800 (i.e., a view along the longitudinal axis of catheter 899 from a distal region). FIG. 8B shows a side view (i.e., a view perpendicular to the longitudinal axis of catheter 899) of heart-tissue-conforming element 800. FIG. 8C shows an isometric view of heart-tissue-conforming element 800. In FIGS. 8A-C, heart-tissue-conforming element 800 comprises a single mid-radius balloon 810, and heart-tissue-conforming element 800 is attached to a distal catheter perfusion section, i.e., a region near the distal end, of catheter 899. Heart-tissue-conforming element 800 further comprises winged balloons 820a, 820b and 820c configured as cylinders evenly spaced around the circumference of mid radius balloon 810. Winged balloons 820a, 820b and 820c are fluidically coupled to small tubes 830a, 830b and 830c extending externally along catheter 899, from which pulsatile energy is transmitted and applied to winged balloons 820a, 820b and 820c via fluid injected into winged balloons 820a, 820b and 820c. Separate tubes 830a, 830b and 830c enable winged balloons 820a, 820b and 820c as well as mid-radius balloon 810 to be independently inflated, i.e., to different magnitudes and at different frequencies and duty cycles. Tubes, such as separate tubes 830a, 830b and 830c, included in embodiments of a system, such as system 100, may be configured to hold and transmit any type of fluid, such as, for example, saline solution or contrast solution or the like. Additionally, in embodiments, tubes, such as separate tubes 830a, 830b and 830c, may comprise optical or electrical cables configured to apply energy to cardiovascular tissue, such as, for example, to generate a cavitation bubble.

Heart-tissue-conforming element 800 is present on distal catheter perfusion section 870 of catheter 899 comprising perfusion entrance zone 850 and perfusion exit zone 860. Perfusion entrance zone 850 and perfusion exit zone 860 are configured to allow blood to perfuse past heart-tissue-conforming element 800. Perfusion entrance zone 850 and perfusion exit zone 860 comprise a plurality of ports configured for blood to flow into and out of via fluidic passages (not shown) connecting perfusion entrance zone 850 to perfusion exit zone 860. That is, heart-tissue-conforming element 800 and/or catheter 899 comprise one or more internal fluidic pathway (not shown) fluidically connecting perfusion entrance zone 850 and perfusion exit zone 860, through which blood flows during treatment using heart-tissue-conforming element 800.

Heart-tissue-conforming element 800 further comprises guidewire entrance port 870 configured to allow a standard guidewire to be threaded into heart-tissue-conforming element 800 through a guidewire channel (not shown) to a guidewire exit port (not shown) at a more proximal region of the catheter. Guidewire entrance port 870 as well as guidewire channel and exit port facilitate guiding heart-tissue-conforming element 800 through relevant anatomy via a standard, over-the-wire approach.

Heart-tissue-conforming element 800 may further comprise one or more markers (not shown), such as radiopaque markers, present on any convenient location of heart-tissue-conforming element 800 configured for use confirming a desired location and/or orientation or alignment of heart-tissue-conforming element 800 relative to, e.g., cardiovascular tissue, e.g., diseased cardiovascular tissue. In some cases, such markers may be configured to verify commissural alignment or alignment with other aspects of a heart valve.

In some cases, aspects of mid-radius balloon 810 and winged balloons 820a, 820b and 820c may be configured to facilitate heart-tissue-conforming element 800 aligning itself, i.e., self-centering, with respect to cardiovascular tissue, such as a heart valve. For example, in some cases, complaint balloons are used for winged balloons 820a, 820b and 820c, and a compliant balloon is used for mid-radius balloon 810; and in other cases, a compliant balloon may be used for only one of or neither of winged balloons 820a, 820b and 820c and mid-radius balloon 810. In such an embodiment, winged balloons 820a, 820b and 820c may be inflated first, followed by inflating the mid-radius balloon 810, which, as it inflates, auto centers the winged balloons 820a, 820b and 820c into heart valve commissures. In other cases, an order of balloon inflation and/or the use of compliant or non-compliant materials in such balloons may be changed depending on the need or nature of the diseased cardiovascular tissue. In still other cases, the balloons of heart tissue conforming element 800 may be inflated, pulsated and deflated/collapsed as follows. First, mid-radius balloon 810 may be inflated in diastole. Simultaneously, winged balloons 820a, 820b and 820c are also inflated and pulsated in diastole. After that, mid-radius balloon 810 as well as winged balloons 820a, 820b and 820c are deflated or collapsed in systole. Embodiments that utilize such an inflation, pulsation and deflation pattern, i.e., in which different balloons are inflated and/or pulsated and/or collapsed at different times, may comprise more than one potential sources, i.e., more than one console assemblies in order to facilitate such inflation patterns.

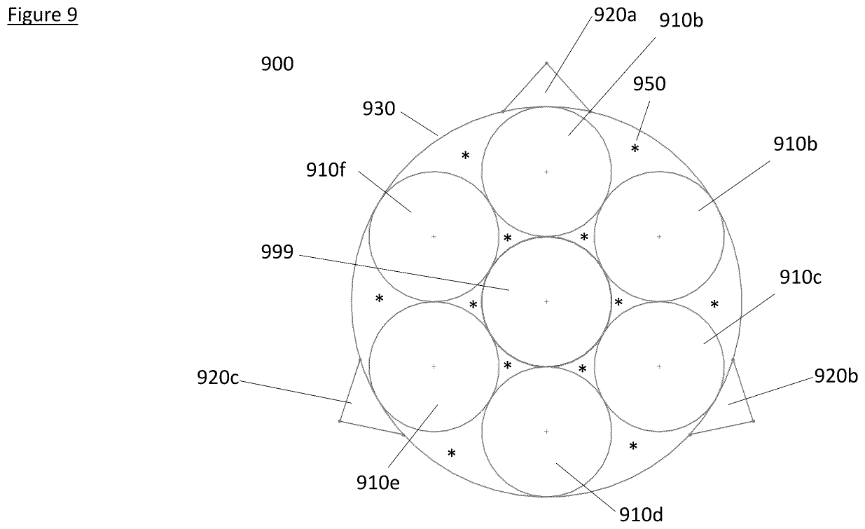

FIG. 9 depicts an exemplary embodiment of a heart-tissue-conforming element 900 according to an embodiment of the invention, e.g., that may be used in a system, such as system 100 schematically illustrated in FIG. 1. FIG. 9 shows a cross sectional view of heart-tissue-conforming element 900 (i.e., a view along the longitudinal axis of heart-tissue-conforming element 900 viewed from a distal region of catheter 999), similar to heart-tissue-conforming element 800 but with a different balloon configuration. In FIG. 9, heart-tissue-conforming element 900 comprises a plurality of mid-radius balloons 910a, 910b, 910c, 910d, 910e and 910f, circumferentially attached and surrounding catheter 999. That is, each of mid-radius balloons 910a, 910b, 910c, 910d, 910e and 910f is attached, i.e., bonded, to its neighbor as well as to catheter 999.

Heart-tissue-conforming element 900 further comprises winged balloons 920a, 920b and 920c present on an external surface. Winged balloons 920a, 920b and 920c are configured such that their cross-section is a triangular shape, evenly spaced around the circumference of heart-tissue-conforming element 900, i.e., such that winged balloons 920a, 920b and 920c are configured to engage heart valve commissures, such as aortic valve commissures while regions of mid-radius balloons 910a, 910b, 910c, 910d, 910e and 910f are configured to engage heart valve leaflets, such as aortic valve leaflets. The long side of the triangular cross section of winged balloons 920a, 920b and 920c are each attached to heart-tissue-conforming element 900. Heart-tissue-conforming element 900 further comprises outer sheath (i.e., outer constraint band) 930 configured to surround and envelope mid-radius balloons 910a, 910b, 910c, 910d, 910e and 910f and to maintain a substantially constant configuration of heart-tissue-conforming element 900. Winged balloons 920a, 920b and 920c are configured such that they are attached to an outer surface of outer sheath 930. Heart-tissue-conforming element 900 comprises a distal catheter perfusion zone of which only perfusion entrance zone comprising a plurality of input ports such as perfusion zone entrance port 950 is depicted in FIG. 9. Perfusion entrance zone, including input ports, such as input port 950, is configured such that blood can enter and traverse past heart-tissue-conforming element 900, ultimately exiting through a perfusion exit zone (not shown) on a relatively proximal region of the catheter assembly, i.e., in order to perfuse distal vessels.

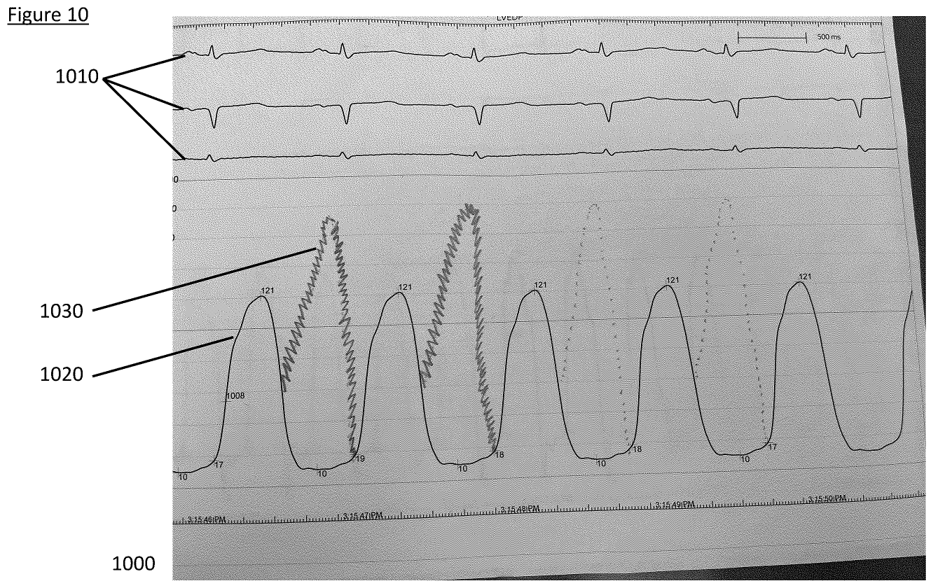

FIG. 10 depicts balloon pressurization (i.e., inflation/deflation) sequences of an exemplary heart-tissue-conforming element that are based on, i.e., coordinated with, electrocardiogram (ECG) readings from a subject receiving treatment using a device according to the present invention. Plot 1000 includes ECG signals 1010 measured from a subject undergoing treatment using a device according to the present invention. Plot 1000 also includes pressure readings 1020, 1030 representing pressure applied to balloons of the heart-tissue-conforming element of the exemplary device of the present invention, showing how pressure is applied at different amplitudes at different times with respect to ECG signals 1010. In the example depicted in FIG. 10, pressures 1020, 1030, in order to conduct a desired treatment using a device of the present invention, are controlled based on the readings of ECG signals 1010, as shown in FIG. 10.

Pressure signal 1020 shows pressure applied to the one or more mid-radius balloons of the heart-tissue-conforming element, such as mid-radius balloon 810 of heart-tissue-conforming element 800 depicted in FIG. 8 or mid-radius balloons 910a, 910b, 910c, 910d, 910e and 910f of heart-tissue-conforming element 900 depicted in FIG. 9.

Pressure signal 1030 shows pressure applied to the one or more winged balloons of the heart-tissue-conforming element, such as winged balloons 820a, 820b and 820c of heart-tissue-conforming element 800 depicted in FIG. 8 or winged balloons 920a, 920b and 920c of heart-tissue-conforming element 900 depicted in FIG. 9.

Pressure signal 1030 is depicted as a squiggly line as well as a dotted line. The squiggly line is used to depict relatively small oscillations (i.e., relatively higher frequency oscillations) over time, i.e., relatively smaller oscillations within the relatively longer oscillations (i.e., relatively lower frequency oscillations) in which pressure 1030 cycles between zero and a maximum pressure. While the shorter time period oscillations (i.e., relatively higher frequency oscillations) seen in the squiggly line of pressure 1030 do not depict large changes in pressure, any convenient pressure changes may be applied in embodiments. For example, the relatively higher frequency pressure changes (i.e., represented by the squiggly line section of pressure 1030) may cycle between zero and a maximum pressure (where such maximum pressure increases and decreases according the relatively longer oscillations (i.e., relatively lower frequency oscillations) of pressure 1030, or any range in between zero and such maximum pressure.

With respect to ECG signals 1010 and pressure signals 1020, 1030, in each case, the x-axis represents time. With respect to ECG signals 1010, the y-axis represents changes in electrical potential. With respect to pressure signals 1020, 1030, the y-axis represents changes in pressure.

As described above, FIG. 10 shows two phases to pressure cycles 1020, 1030. As described above, pressure 1020 corresponds to the main inflation of the one or more mid-radius balloons and goes up and down in diastole (starts as the heart is relaxing and then the one or more mid-radius balloons collapse before the heart contracts again). As described above, pressure 1030 has jagged edges (i.e., the squiggly line) representing the faster ongoing oscillatory waves generated by the secondary structures (e.g., winged balloons) that are attached to the one or more mid-radius balloons. Applying pressures 1020, 1030 substantially as shown in FIG. 10 will have the effect of causing the heart-tissue-conforming element to better engage the space between the heart valve leaflets and, in some cases, will affect treatment time.

While FIG. 10 shows pressures 1020, 1030 applied during specific times associated with specific aspects or features of ECG signals 1010, any convenient coordination between pressures 1020, 1030 and ECG signals 1010 may be applied and such may vary, for example, depending on the desired treatment to be applied to a subject or characteristics of the subject or any other relevant consideration.

FIG. 10 shows pressures 1020, 1030 being relatively out of phase with each other for purposes of better illustrating the two pressure curves 1020, 1030. However, any convenient phase (i.e., distance in time between relative maximums of pressure curves 1020, 1030) may be applied. For example, pressures 1020 and 1030 may achieve relative maximum pressures, respectively, at the same time in some cases. In other cases, pressure 1020 may achieve relative maximum pressure at the same time that pressure 1030 is at a minimum pressure, e.g., zero pressure. In still other cases, pressure 1030 may achieve relative maximum pressure at the same time pressure 1020 is at a minimum pressure, e.g., zero pressure. Similarly, pressures 1020, 1030 may exhibit the same frequencies (i.e., with respect to the lower frequency of pressure 1030 shown in FIG. 10) or different frequencies, as desired, and may be separately and independently controlled using the same or different potential sources and/or oscillators, as desired, in order to achieve the desired relative characteristics of pressures 1020, 1030.

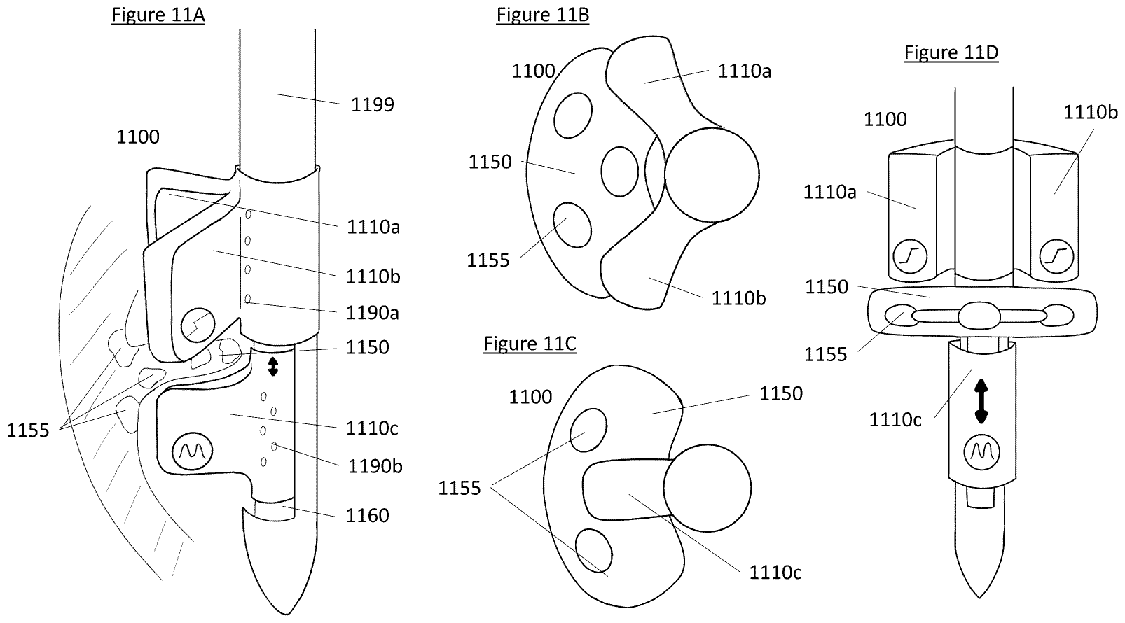

FIGS. 11A to 11D depict multiple views of an alternative radial-member-based embodiment of a heart-tissue-conforming element 1100. FIG. 11A shows an isometric view of heart-tissue-conforming element 1100. FIG. 11B shows a top view (i.e., a view along the longitudinal axis of catheter 1199 from a proximal region) of heart-tissue-conforming element 1100. FIG. 11C shows a bottom view (i.e., a view along the longitudinal axis of catheter 1199 from a distal region) of heart-tissue-conforming element 1100. FIG. 11D shows a side view of heart-tissue-conforming element 1100. In FIGS. 11A-D, heart-tissue-conforming element comprises radial members 1110a, 1110b and 1110c, and heart-tissue-conforming element 1100 is attached to a distal region, near the distal end, of catheter 1199. In FIGS. 11A-D, heart-tissue-conforming element 1100 is shown engaging with cardiovascular tissue, i.e., heart valve leaflet 1150 with calcifications, i.e., calcium deposits 1155.

Heart-tissue-conforming element 1100 depicted in FIGS. 11A-D comprises radial members 1110a and 1110b that are eccentric balloons located at a relatively proximal location on catheter 1199, i.e., at substantially the same distance from the distal end of catheter 1199. While heart-tissue-conforming element 1100 shown in FIGS. 11A-D comprises two such radial members 1110a and 1110b, other embodiments may comprise one or greater than two such radial members. Upon inflation of the balloons that comprise radial members 1110a and 1110b, the distal ends of radial members 1110a and 1110b span a side of valve leaflet 1150, i.e., the aortic side of valve leaflet 1150. Radial members 1110a and 1110b, comprising balloons, are configured to be inflated such that they provide a significant resistive force to the top (i.e., proximal side) of valve leaflet 1150.

Heart-tissue-conforming element 1100 depicted in FIGS. 11A-D further comprises radial member 1110c that is an eccentric balloon attached at a relatively distal location of catheter 1199. While heart-tissue-conforming element 1100 shown in FIGS. 11A-D comprises one such radial members 1110c, other embodiments may comprise one or greater than one such radial members. When inflated, the proximal end of radial member 1110c comprising a balloon will engage, for example, the left ventricular side of heart valve leaflet 1150. FIGS. 11B and 11C depict how radial member 1110c is located in a position that substantially bisects the angle formed between radial members 1110a and 1110b. Porting holes 1190a comprise fluidic connections between fluidic passages of catheter 1199 and the balloons of radial members 1110a and 1110b such that fluid can be displaced into balloons of radial members 1110a and 1110b, causing radial members 1110a and 1110b to expand, including to apply pulsatile energy to cardiovascular tissue. Porting holes 1190b comprise a fluidic connection between a fluidic passage of catheter 1199 and the balloon of radial member 1110c such that fluid can be displaced into balloon of radial member 1110c, causing radial member 1110c to expand, including to apply pulsatile energy to cardiovascular tissue.

When the balloons of radial members 1110a, 1110b and 1110c are inflated, such balloons act to create a three-point flexure condition in which the heart valve leaflet 1150 and the embedded calcium 1155 are flexed, compressed, or tensioned. That is, leaflet 1150 is urged in a proximal direction by expansion of radial member 1110c and simultaneously urged in a distal direction by expansion of radial members 1110a and 1110b. Sustained and/or pulsatile pressure may be applied to the balloons of radial members 1110a, 1110b and 1110c to controllably fracture internal calcium 1155 of leaflet 1150.

In certain instances, radial member 1110c comprising a distal eccentric balloon may be attached to an inner shaft 1160 of catheter 1199 that is configured to translate axially (i.e., along the long access of catheter 1199). When radial member 1110c is translated in a proximal direction, the distal eccentric balloon of radial member 1110c can further engulf or encompass or engage with valve leaflet 1150 (i.e., the distal side of leaflet 1150) such that a secure connection may be made between the proximal and distal eccentric balloons, i.e., between radial members 1110a, 1110b and radial member 1110c.

In some instances, a portion of a radial member comprising a balloon, such as radial member 1110a, 1110b or 1110c, for example, that is in contact with cardiovascular tissue, e.g., a heart valve, such as heart valve leaflet 1150, may be compliant, or semi-compliant, such that applying increased pressure to the balloon causes a distension of the balloon into the valve leaflet. This additional distension increases the force applied to the heart valve leaflet, e.g., in a bending configuration, which force increases the ability to fracture internal calcifications, such as calcifications 1155, of the cardiovascular tissue, e.g., a heart valve, such as heart valve leaflet 1150. In other instances, a portion of a radial member comprising a balloon, such as radial member 1110a, 1110b or 1110c, for example, that is in contact with cardiovascular tissue, e.g., a heart valve, such as heart valve leaflet 1150, may be configured to have ridges or cutting or scoring elements such that, when in contact with cardiovascular tissue, such as heart valve leaflet 1150, the edges of these features generate stress concentrations in the calcium of the cardiovascular tissue, e.g., heart valve leaflet 1150. Such stress concentrations may work to generate increased calcium fracturing while at the same time reducing the required pressure needed to apply to the balloon of a radial member. Similarly, the portion of the balloon of a radial member in contact with cardiovascular tissue, such as a proximal or distal side of radial members 1110a, 1110b and 1110c in contact with heart valve leaflet 1150, may be configured to elute an active agent, such as a drug, such that, when in contact with the cardiovascular tissue, the active agent is eluted onto the heart valve leaflet to minimize restenosis and increase the longevity of valve treatment.

In other embodiments, various numbers of radial members may be located around the axis of the catheter, e.g., catheter 1199, such that the heart-tissue-conforming element is configured to treat one or more heart valve leaflets at the same time. It may be appreciated that in the former instance, i.e., when the heart-valve-conforming element is configured to treat one heart valve leaflet at a time, blood may still be able to perfuse through the heart valve on via another side of the catheter, e.g., a side of the catheter opposite of the radial members, thereby extending treatment time. In the latter instance, i.e., when the heart-valve-conforming element is configured to treat more than one heart valve leaflet at a time, such configuration may cause blood to be restricted to a greater degree than when the heart-valve-conforming element is configured to treat one heart valve leaflet at a time.

In some embodiments, the catheter, e.g., catheter 1199, may comprise a port (not shown) configured to apply vacuum to a nearby region of cardiovascular tissue such that the port engages with the cardiovascular tissue and holds the leaflet in a fixed location during treatment.

In some instances, the faces of the radial members or balloons thereof that contact cardiovascular tissue, such as a heart valve leaflet, are shaped according to, for example, a profile of the heart valve leaflet. For example, the faces of the radial members or balloons thereof that contact cardiovascular tissue may be shaped such that they are convex on the aortic side and concave on the ventricular side. For example, distal faces of radial members 1110a and 1110b (i.e., the surface in contact with leaflet 1150) may have a substantially concave shape, whereas distal face of radial member 1110c (i.e., the surface in contact with leaflet 1150) may have a substantially convex shape.

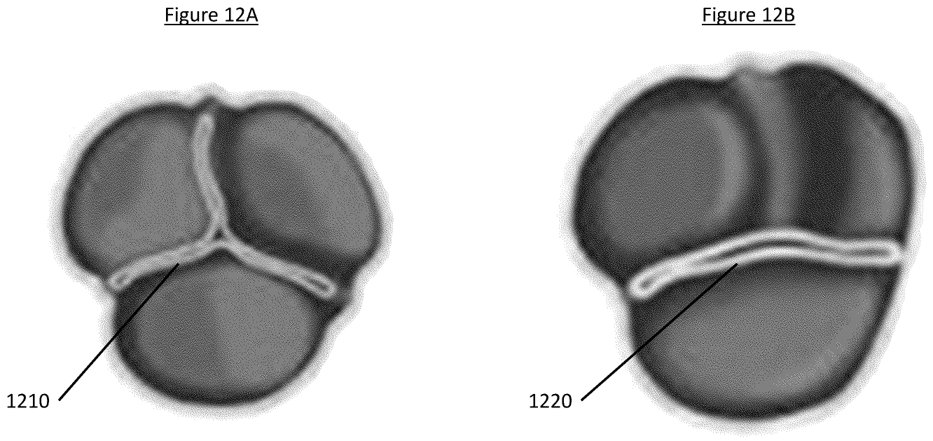

FIGS. 12A and 12B depict cross sectional views of heart valves, such as heart valves that may receive treatment using embodiments of the present invention. FIGS. 12A-B depict trileaflet valve 1210 as well as a bicuspid valve or bileaflet valve 1220. The mitral valve is an example of a bicuspid valve or bileaflet valve. As described herein, some embodiments of heart-tissue-conforming elements according to the present invention may be tailored for treatment of trileaflet valves, such as trileaflet valve 1210, whereas other embodiments of heart-tissue-conforming elements according to the present invention may be tailored for treatment of bileaflet valves, such as bileaflet valve 1210. For example, in some cases, better outcomes are achieved with bicuspid valves or bileaflet valves if such valves are treated with heart-tissue-conforming elements (or one or more mid-radius balloons thereof) that is wider in one dimension compared to a circular cross section of a heart-tissue-conforming element: that is, a heart-tissue-conforming element that has an oblong cross section or a heart-tissue-conforming element that has an oval-shaped cross section.

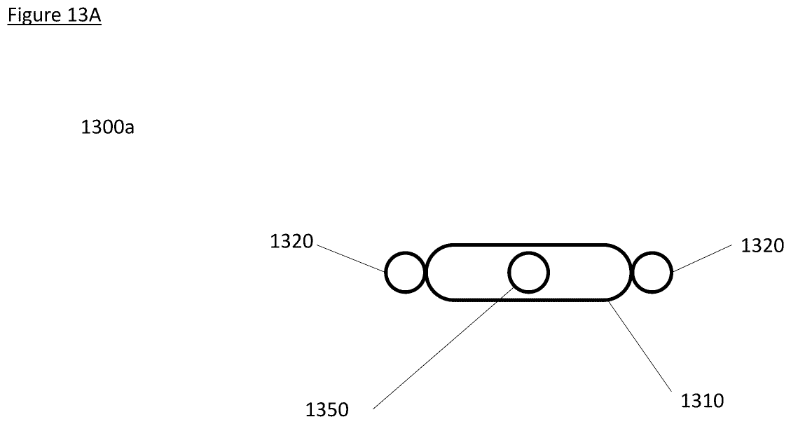

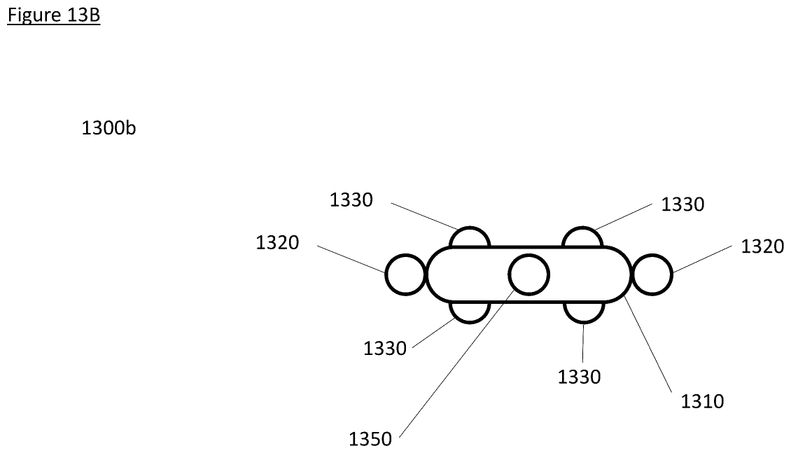

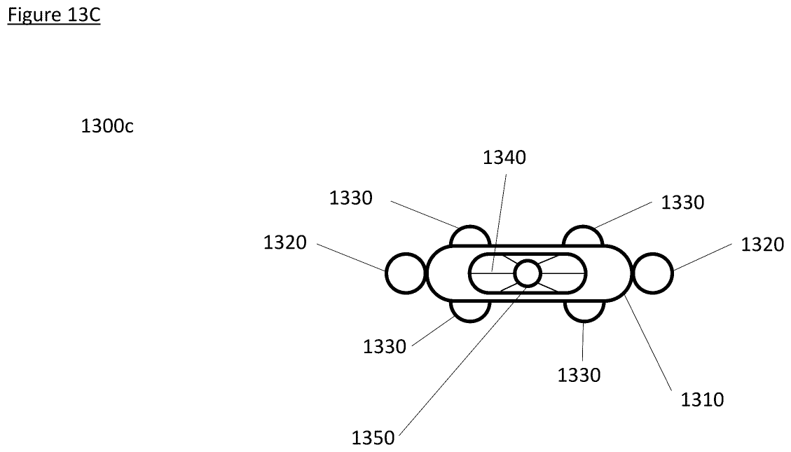

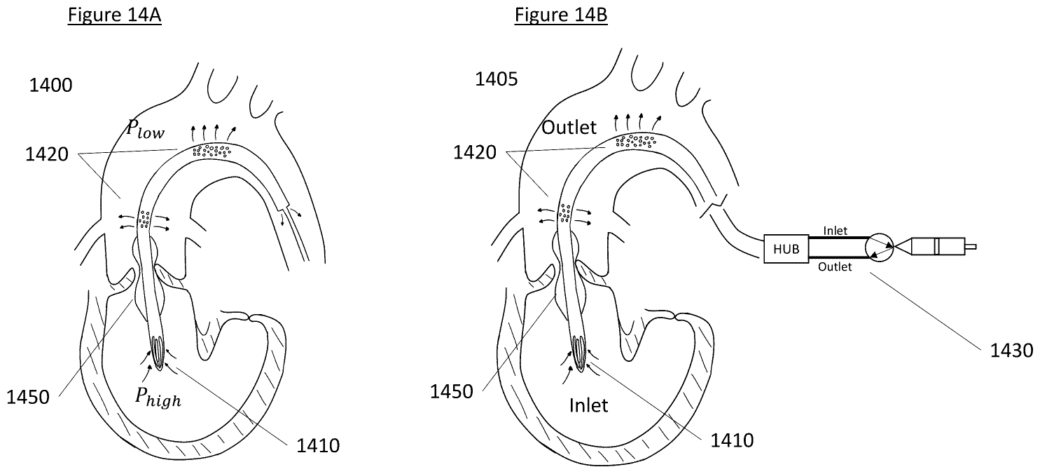

FIGS. 13A-C depict embodiments of heart-tissue-conforming elements that have a cross sectional shape that is wider in one dimension compared to a circular cross sectional shape of a heart-tissue-conforming element. Heart-tissue-conforming element 1300a in FIG. 13A is shown in cross section. Heart-tissue-conforming element 1300a comprises mid-radius balloon 1310 that is substantially oblong or oval shaped with winged balloons 1320 attached to opposite ends of mid-radius balloon 1310. Mid-radius balloon 1310 has an outward expansion and may be inflated and deflated, i.e., pulsated, at a certain frequency (i.e., mid-radius balloon 1310 may have pulsatility of a certain frequency), and winged balloons 1320 on the side of heart-tissue-conforming element may be inflated and deflated, i.e., pulsated, at a different or same frequency. Heart-tissue-conforming element 1300a comprises a cross-sectional shape such that it will preferentially expand along the natural commissure of the valve (i.e., bicuspid or bileaflet valve, such as bileaflet valve 1210 depicted in FIG. 12B) and expand along the natural fusion line compared with embodiments of heart-tissue-conforming elements (or balloons thereof) that expand in all directions equally. The center of heart-tissue-conforming element 1300a comprises wire channel 1350, e.g., for use with a guidewire.

FIG. 13B presents another embodiment of a heart-tissue-conforming element 1300b with cross-sectional shape configured for use with a bicuspid or bileaflet valve, such as bileaflet valve 1210 depicted in FIG. 12B. Aspects of heart-tissue-conforming element 1300b that are identical of heart-tissue-conforming element 1300a are not repeated here. Heart-tissue-conforming element 1300b comprises additional winged balloons 1330 present on the oblong, or relatively elongated, sides of heart-tissue-conforming element 1300b. Additional winged balloons 1330 may be pulsated at the same or different frequencies as mid-radius balloon 1310 and winged balloons 1320.

FIG. 13C presents another embodiment of a heart-tissue-conforming element 1300c with cross-sectional shape configured for use with a bicuspid or bileaflet valve, such as bileaflet valve 1210 depicted in FIG. 12B. Aspects of heart-tissue-conforming element 1300c that are identical of heart-tissue-conforming elements 1300a and 1300b are not repeated here. Heart-tissue-conforming element 1300c comprises perfusion zone 1340 (or perfusion path or central channel), part of a perfusion mechanism of heart-tissue-conforming element 1300c, with wire cage that maintains the shape of perfusion zone 1340 (i.e., keeps perfusion zone 1340 open) during treatment, even as mid-radius balloon 1310 is pressurized and expanded. Perfusion zone 1340 is used to allow fluid, i.e., blood, to perfuse past heart-tissue-conforming element 1300c during treatment, and the cage of perfusion zone 1340 maintains structural stability of the opening during treatment, preventing perfusion zone 1340 to collapse even when mid-radius balloon 1310 is pressurized. Perfusion zone 1340 is shown in cross section in FIG. 13C such that entrance and exits paths are not depicted in the figure, but such are fluidically interconnected via the cross-sectional path shown in FIG. 13C.

FIG. 14A illustrates a passive perfusion mechanism 1400 past heart-tissue-conforming element 1450, where perfusion is enabled by a prevailing relatively high pressure ventricular region (P_{high}) and a prevailing relatively low pressure aortic region (P_{low}). The relatively high pressure ventricular region urges blood into perfusion entrance zone 1410 comprising a plurality of ports and out perfusion exit zone 1420 comprising a plurality of ports via a fluidic channel (not shown) past heart-tissue-conforming element 1450.

FIG. 14B illustrates an active perfusion mechanism 1405 past heart-tissue-conforming element 1450, where perfusion is facilitated by perfusion pump 1430 configured to pull blood from perfusion entrance zone 1410 comprising a plurality of ports and push blood to perfusion exit zone 1420 comprising a plurality of ports. Perfusion pump 1430 utilizes a continuous reciprocating motion of a barrel-like syringe connector to pull fluid, i.e., blood, from perfusion entrance zone 1410 and push fluid out perfusion exit port 1420.

FIG. 15 depicts a control loop schematic for controlling the procedure for treating diseased cardiovascular tissue, such as a diseased valve, using embodiments of the current invention. In this embodiment, the controller includes a treatment plan that is fed into the controller subsystem as input. The controller subsystem sends signals such as in the form of voltage or data to the potential source (regulated or unregulated) to control the amount of potential output by the potential source to the oscillator. The controller subsystem also sends signals to the oscillator, such as frequency (f), i.e., oscillation frequency, and duty cycle (tₒₙ and t_{off}), to control the cyclical energy transmitted by the oscillator. Feedforward models of the individual subsystems (e.g., console assembly, manifold assembly, catheter assembly) or the system as a whole, may be injected into the controller signal (i.e., the controller signal may be augmented with such data regarding a feedforward model) to improve the convergence of the controller (i.e., a control algorithm) to the correctly supplied energy.

Therapy energy (i.e., potential energy) is transmitted to the oscillator, but sensors may be used to track the magnitude of potential energy transmitted to the oscillator. The sensor signals may be fed back into the controller such that they may be used to converge the output of system (i.e., pulsatile energy applied to cardiovascular tissue) to the desired output. The oscillator is turned on and off at an appropriate frequency and duty cycle such that energy is transmitted in an appropriate fashion to the distal location (i.e., to the heart-tissue-conforming element and ultimately to cardiovascular tissue), accounting for any attenuation, heat transfer, bubble formation, tissue relaxation, and the like. Sensors measure system attributes, as well as characteristics of the cardiovascular tissue, such as pressure, volume, temperature, flow and the like. Sensor data from the catheter assembly are fed back into the controller so that such measurements may be used to converge the output of the system (i.e., pulsatile energy applied to cardiovascular tissue) to the desired output. The catheter assembly sensors may be located at any convenient location of the catheter assembly where they can be configured to produce an appropriate measurement that can be used for controlling the system. For instance, the sensors may be pressure or volume sensors located on a connector. In other instances, such sensors may be sensors located in the catheter or heart-tissue-conforming element (e.g., in a balloon of the heart-tissue-conforming element) and are connected to wires configured to pass through the catheter. In other cases, such sensors may comprise an X-ray image of the heart-tissue-conforming element, which is used to determine position or expansion of one or more balloons of the heart-tissue-conforming element.

The catheter assembly may comprise one or more sensors configured to measure information at one location as a means for determining the conditions elsewhere. For instance, a pressure and flow transducer may be configured to measure the pressure and flow rate at the connector, but, for example, combined with a fluid model of the system and X-ray or CT imaging, the sensor data may be used to gauge the pressure and/or volume of a balloon of the heart-tissue-conforming element. In addition, sensor data may be provided to a clinician feedback transfer function, where such sensor feedback may be combined with feedback generated from the potential transfer plant or imaging data, for processing to a feedback mechanism plant for providing feedback on system behavior to a provider, i.e., operator of the system.

In other instances, a pressure and flow rate sensor may be used to measure the compliance or change in compliance of a balloon of the heart-tissue-conforming element. Given that diseased tissue provides a resistance to balloon expansion, the amount of volume forced into the balloon at a certain pressure is an indirect indication of the compliance of the surrounding tissue. In this way, a measure of compliance or change in compliance may be used as a treatment metric or goal.

FIG. 16 depicts a schematic of a system configuration 1600 for a robotic method for delivering therapy energy to a diseased valve from a control room. As described above, the embodiment of the system may be partitioned into physically separate control room 1610 and treatment location 1620, which may be separated, e.g., in the next room or in different cities or states or countries.

Control room 1610 may comprise a console display, an imaging display, a treatment controller, a device position controller, patient information as well as a connection to a procedural database. Console displays may be used to provide important information about the state of the system relevant to an ongoing treatment, such as, for example, pressure or volume measurements or other information about a heart-tissue-conforming element, such as the type of heart-tissue-conforming element employed. Information provided on the Console displays may be updated periodically during treatment, e.g., substantially in real time. Imaging displays may be used to provide imaging information, such as fluoroscopy imaging results, e.g., showing the position of the heart-tissue-conforming element relative to cardiovascular tissue, e.g., a heart valve. Fluoroscopy imaging results may also provide information about the state of aspects of the heart-tissue-conforming element, such as, e.g., whether a balloon is inflated and to what extent. Treatment controller may be used to adjust an aspect of how the system is applied to provide treatment, e.g., apply pulsatile energy to the cardiovascular tissue, such as, for example, adjusting a pressure or frequency or duty cycle setting. Device position controller may be used to adjust the position of the device relative to cardiovascular tissue undergoing treatment, e.g., make adjustments to a catheter position to move the heart-tissue-conforming more proximally or distally. Patient information may comprise a display of any important patient data relevant to a treatment, such as, for example, information about a treatment site, a patient age, disease state, vitals measurements, such as blood pressure or pulse oximeter readings. Procedural database comprises relevant information regarding past treatment plans (i.e., information related to system configurations for treating cardiovascular tissue, such as oscillation frequencies, duty cycles and/or amplitudes and corresponding details regarding the underlying cardiovascular tissue, such as a degree of calcification), which may be accessed to identify a potential treatment plan in connection with a new treatment.

Treatment location 1620 may comprise a system according to the present invention, i.e., an embodiment of a system comprising a console assembly with a potential source, a manifold assembly and a catheter assembly with a connector, a connector-to-catheter transition hub, a catheter and a heart-tissue-conforming element. Treatment room 1620 may be, for example, a procedure room or operating room. Any convenient operably connection capable of transmitting data and control signals between treatment room 1620 and control room 1610 may be employed, such as a wired or wireless connection.

### Measurements of Compliance:

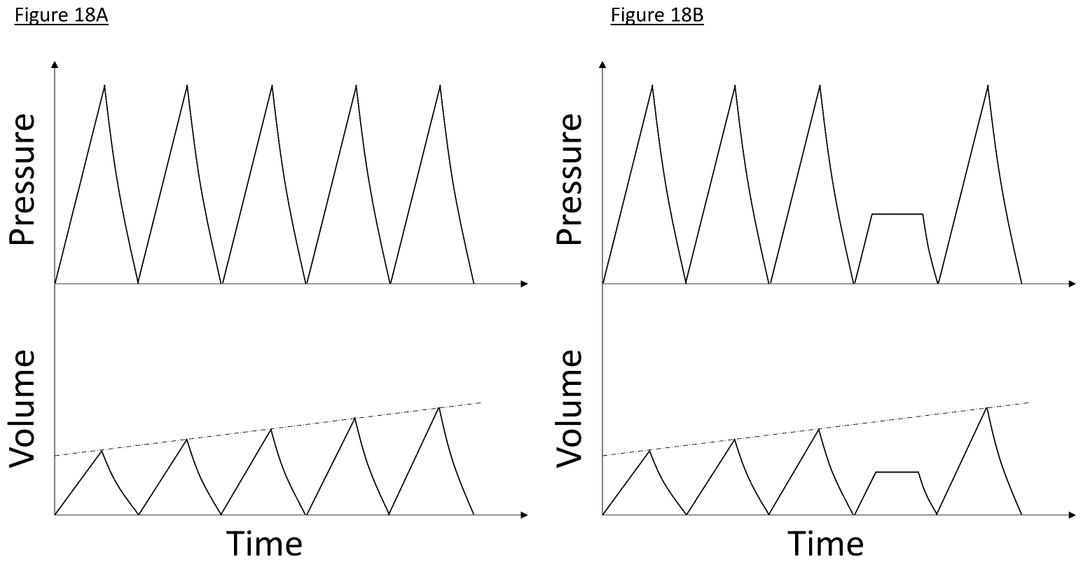

As described in detail below, tissue, e.g., cardiovascular tissue or vessel, compliance is a measurable characteristic of blood vessels or cardiovascular tissue, such as cardiovascular tissue comprising a heart valve, calculated based on a ratio of the change in tissue volume for a given change in pressure. Vessel compliance is an important characteristic for observation because improving vessel compliance is a prerequisite to definitive treatment of certain underlying disease conditions, such as atherosclerosis or the presence of calcifications in cardiovascular tissue. Changes in vessel compliance are seen in the different pressure-volume curves depicted in FIG. 17. In FIG. 17, volume is plotted on the x axis and pressure is plotted on the y axis. The pressure-volume, i.e., vessel compliance, characteristic of an unrestrained balloon (i.e., a balloon that is not present in cardiovascular tissue or otherwise restrained from expanding its volume upon increase in balloon pressure) versus untreated (i.e., pre-treatment) cardiovascular tissue, i.e., an untreated vessel, versus treated (i.e., post-treatment) cardiovascular tissue, i.e., a treated vessel.

Shown in FIG. 17, upon treatment, e.g., application of pulsatile energy to cardiovascular tissue using an embodiment of a system of the present invention, the pressure-volume curve shifts to the right, closer to the curve of the unrestrained balloon. That is, upon treatment, an identical change in tissue volume corresponds to a reduced tissue pressure; i.e., it takes less pressure on the tissue to expand tissue volume a similar amount.

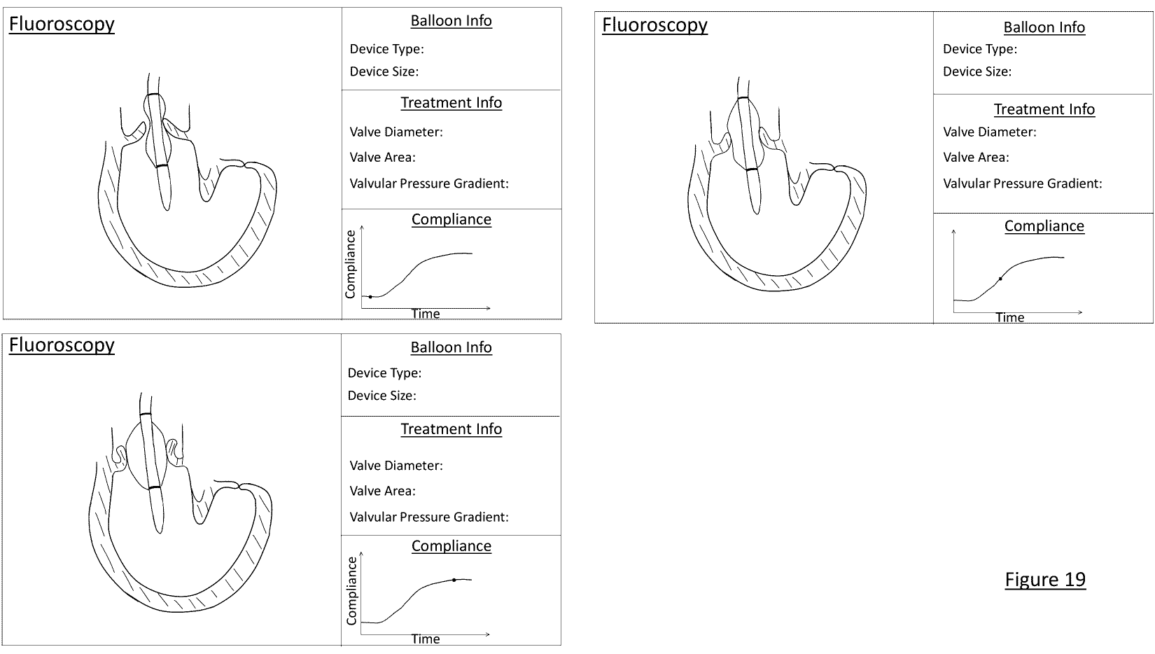

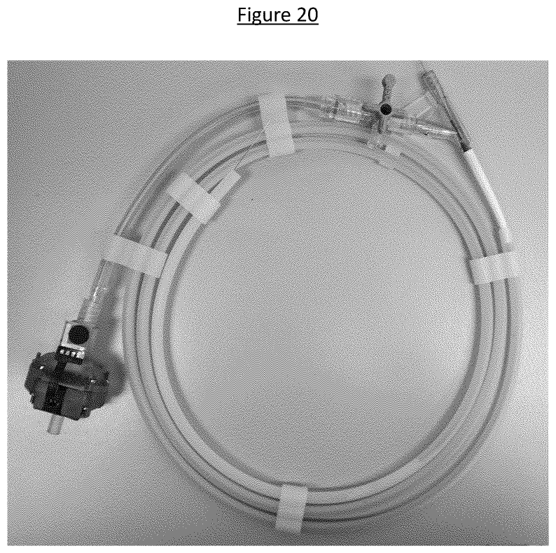

Systems according to the present invention may be configured to assess tissue, e.g., vessel, compliance by obtaining measurements in-vivo of changes in volume at different pressures (or different changes in pressure) applied to cardiovascular tissue such as vessels. FIGS. 18A and 18B provide an example of measurements of changes in tissue compliance obtained during treatment using a system according to the present invention, i.e., during pulsatile lithotripsy within a heart valve. FIG. 18A demonstrates obtaining measurements of tissue compliance, i.e., pressure applied to tissue and corresponding changes in volume over time, using dynamic changes in pressure. FIG. 18A shows peak volume changes trend upward while peak applied pressure remains constant. That is, as treatment progresses, tissue volume increases to a greater extent while the same changes in pressure are applied, an indication of improved tissue compliance.

FIG. 18B demonstrates obtaining measurements of tissue compliance, i.e., pressure applied to tissue and corresponding changes in volume over time, using dynamic changes in pressure as well as static pressure (i.e., on the third pressure oscillation the pressure applied to tissue remains for approximately half the cycle). FIG. 18B shows peak volume changes trend upward while peak applied pressure remains constant. That is, as treatment progresses, tissue volume increases to a greater extent while the same changes in pressure are applied, an indication of improved tissue compliance.

Embodiments of the present invention enable measurement of relative compliance change of cardiovascular tissue (e.g., a vessel) in real time during application of a system of the present invention to provide pulsatile energy to cardiovascular tissue. Systems of the present invention may be configured to measure, and update, treatment parameters based on compliance change of the cardiovascular tissue. For instance, after calcium cracking (i.e., breaking up of calcified plaque tissue), the cardiovascular tissue and heart-tissue-conforming element or a balloon thereof may expand significantly, contributing to a large gain in compliance, as measured by the system. However, after the vessel has fully expanded, changes in compliance, as measured by the system, may subside. Identification of such conditions (i.e., different degrees of changes in compliance) may indicate that treatment may be halted because no further appreciable gain is occurring.

Systems may be configured to measure pressure in any convenient manner. In some instances, embodiments of systems according to the present invention may comprise a pressure gauge as described herein for measuring pressure in, for example, fluid passages and/or a balloon of the heart-tissue-conforming element or a catheter of the catheter assembly. In some instances, a pressure gauge may be installed such that it is configured to measure pressures of a distal chamber of a proximal connector, as seen, for example, in pressure transducer 625 in FIG. 6A.

Systems may be configured to measure changes in volume of cardiovascular tissue, e.g., a vessel, in any convenient manner. In some instances, embodiments of systems according to the present invention may be configured such that changes in the position of a membrane (such as membrane 630 in FIG. 6A) separating proximal and distal chambers of a connector (such as connector 600 in FIG. 6A) reflect changes in volume of an aspect, e.g., a balloon, of a heart-tissue-conforming element. Changes in the volume of a balloon reflect changes in the cross-sectional area of cardiovascular tissue, such as a vessel, and therefore changes in volume of the cardiovascular tissue. Such embodiments may further comprise a Hall sensor and a permanent magnet for measuring changes in the position of such a membrane. A Hall sensor refers to a sensor configured to sense the presence of, or changes in, a magnetic field, i.e., by use of the Hall Effect. A permanent magnet may be comprised of any convenient magnetic material, or an electromagnet, as desired, such that relative changes in position of the Hall sensor with respect to the permanent magnet are detected by the Hall sensor. Sensors such as the Hall sensor and permanent magnet described above may be used to measure changes in volume of the heart-tissue-conforming element, or one or more balloons thereof, as well as the rate at which aspects of the heart-tissue-conforming element, e.g., one or more balloons thereof, inflates, i.e., the rate of cardiovascular tissue volume change and corresponding changes in compliance.

### Methods

Methods of imparting pulsatile energy to cardiovascular tissue are also provided and similarly find benefit in the applications described above. Methods according to the present invention comprise deploying a system so that a heart-tissue-conforming element of the system is adjacent to cardiovascular tissue. Such system comprises: a console assembly comprising a potential source; a manifold assembly operably connected to an output of the console assembly, wherein the manifold assembly comprises an oscillator configured to generate pulse energy from energy transmitted from the potential source; and a catheter assembly operably connected to an output of the manifold assembly, wherein the catheter assembly comprises: a connector operably connecting the catheter assembly to the manifold assembly and configured to transduce a first pulse energy generated by the manifold assembly to a second pulse energy; (ii) a catheter comprising a fluidic passage operably connected to the output of the connector and configured to transmit the second pulse energy; and the heart-tissue-conforming element configured to receive the second pulse energy transmitted through the fluidic passage of the catheter to apply pulsatile energy to the cardiovascular tissue. Such components of systems are described in detail above. Methods according to the present invention further comprise engaging the system in a manner that the heart-tissue-conforming element imparts energy to the cardiovascular tissue. By “imparts energy to the cardiovascular tissue,” it is meant that the heart-tissue-conforming element applies pulsatile energy to the cardiovascular tissue, i.e., sufficient to disrupt calcium formations. For example, the heart-tissue-conforming element may be configured to engage a desired feature of cardiovascular tissue and aspects of the heart-tissue-conforming element, e.g., balloons that comprise the heart-tissue-conforming element, are repeatedly inflated and deflated as described in detail above in connection with embodiments of systems of the invention. Systems may be configured so that different types and degrees of energy are applied to different aspects of cardiovascular tissue, e.g., as between heart valve leaflets and heart valve commissures. Any suitable frequency of inflating and deflating may be used and may vary. In embodiments where the heart-tissue-conforming element comprises a plurality of balloons (or other moving members for imparting pulsatile energy to cardiovascular tissue), the frequency, as well as the amplitude and duty cycle, of inflating and deflating may differ among such balloons or other moving members.

In embodiments, engaging the system in a manner that the heart-tissue-conforming element imparts energy to cardiovascular tissue comprises imparting energy to heart valve tissue, such as, for example, a heart valve leaflet, a heart valve commissure, a heart valve leaflet nodule or a heart valve annulus. For example, in embodiments, engaging the system in a manner that the heart-tissue-conforming element imparts energy to the cardiovascular tissue comprises imparting energy to an aortic valve, such as, for example, an aortic valve leaflet, an aortic valve commissure or an aortic annulus. In other embodiments, engaging the system in a manner that the heart-tissue-conforming element imparts energy to the cardiovascular tissue comprises imparting energy to cardiovascular tissue comprising tissue supporting a heart valve. In still other embodiments, engaging the system in a manner that the heart-tissue-conforming element imparts energy to the cardiovascular tissue comprises imparting energy to an interatrial septum or to an interventricular septum. In such embodiments, the heart-tissue-conforming element may be configured such that upon the heart-tissue-conforming element receiving the second pulse energy transmitted through a fluidic passage of the catheter, features of the heart-tissue-conforming element apply pulsatile energy primarily to specific regions of cardiovascular tissue. For example, an embodiment of a heart-tissue-conforming element may be configured such that a balloon engages with a heart valve commissure so that energy applied to the heart-tissue-conforming element is transmitted to the heart valve commissure.

The system may be configured so that the heart-tissue-conforming element applies different forms or degrees or types of energy to different aspects of cardiovascular tissue. For example, the heart-tissue-conforming element may be configured so that valve commissures receive pulsatile energy at a first frequency, magnitude, duty cycle and/or duration and valve leaflets receive pulsatile energy at a second frequency, magnitude, duty cycle and/or duration. That is, applying a system of the invention to treat cardiovascular tissue may comprise treating different aspects of cardiovascular tissue differently.

In embodiments, engaging the system in a manner that the heart-tissue-conforming element imparts energy to the cardiovascular tissue comprises engaging the system in a manner to allows fluid to flow past a distal region of the system. By “fluid flowing past a distal region of the system,” it is meant fluid can be displaced from a relatively distal region of the system, e.g., distal to the heart-tissue-conforming element, to a relatively proximal region of the system, e.g., proximal to the heart-tissue-conforming element. In such embodiments, engaging the system causes the heart-tissue-conforming element to impart energy to the cardiovascular tissue while simultaneously allowing fluid, e.g., blood, to perfuse past the heart-tissue-conforming element.

In some embodiments, engaging the system in a manner that the heart-tissue-conforming element imparts energy to the cardiovascular tissue comprises engaging the system in a manner that mimics heart valve function. That is, in embodiments, engaging the system may cause an area of cardiovascular tissue to be substantially sealed or closed or blocked in the same way that a functioning heart valve would cause that area of cardiovascular tissue to be substantially sealed or closed or blocked with respect to the flow of fluid, e.g., blood. In some embodiments, engaging the system causes the heart-tissue-conforming element to substantially behave as a heart valve, for example, while the system is used to apply energy to cardiovascular tissue, such as a heart valve. In some cases, engaging the system in a manner that the heart-tissue-conforming element imparts energy to the cardiovascular tissue comprises engaging the system in a manner that is synchronized with results of an electrocardiogram. For example, in such cases, the heart-tissue-conforming element is configured to behave substantially as a heart valve by sealing or closing or blocking an area of cardiovascular tissue at a rate determined by results of an electrocardiogram.

In other embodiments, engaging the system in a manner that the heart-tissue-conforming element imparts energy to the cardiovascular tissue comprises adjusting a configuration of the system based on procedure-based feedback. Any desired procedure-based feedback may be applied, such as, for example, results of sensors deployed on the system, including, for example, a pressure sensor or a volume sensor for measuring fluid pressure applied to a balloon or fluid volume change of fluid used to inflate a balloon. In some embodiments, “adjusting a configuration of the system,” means adjusting one or more of a fluid pressure or volume or a frequency or a duty cycle of energy applied to the cardiovascular tissue, i.e., a frequency or a duty cycle of the oscillator. In an embodiment, a method of imparting pulsatile energy to cardiovascular tissue is a method for treating cardiovascular tissue, such as a diseased valve, which relies on feedback received during the treatment procedure. Such a method may rely on capturing data from internal pressure and volume sensors during treatment such that valve compliance may be measured before, during and after treatment. In addition to internal sensors, external sensors such as pressure transducers, imaging, temperature sensors, and the like may be utilized. Further, sensors may be provided such that they may be integrated together to operate and provide information at varying frequencies. In addition to feedback sensors, feedforward information such as models of the physics of an embodiment of a system of the invention may be fed into the feedforward system. Such a feedforward system allows further control of the system through enhanced modeling of the system in combination with the feedback sensors.

During treatment according to a method of the present invention, information from various sensors internal and/or external to the system may be read, stored and/or analyzed by the system or such data may be transmitted external to the system to be analyzed at another location such as a cloud cluster. In other instances, the system may be configured to transmit such data, e.g., sensor data, through a learning algorithm to compare it to other instances where a similar and/or equivalent treatment scenario was encountered.

In embodiments, deploying the system so that a heart-tissue-conforming element of the system is adjacent to the cardiovascular tissue comprises aligning the heart-tissue-conforming element with a feature of the cardiovascular tissue. In some cases, the heart-tissue-conforming element is configured to align with specific features of cardiovascular tissue, i.e., by including passive or dynamic elements configured to align with or engage with or hold a fixed position relative to features of cardiovascular tissue. In some cases, the order in which balloons of the heart-tissue-conforming element are inflated facilitates aligning the heart-tissue-conforming element as desired, e.g., such that outer balloons align with commissures of a heart valve. For example, one or more outer, e.g., winged, balloons may be inflated prior to inflating a mid-radius balloon in order to align the heart-tissue conforming element as desired, e.g., with respect to valve commissures. In some cases, the heart-tissue-conforming element comprises lobes for engaging a feature of the cardiovascular tissue, and deploying the system comprises aligning the lobes with the feature of the cardiovascular tissue. In other embodiments, deploying the system so that the heart-tissue-conforming element is adjacent to the cardiovascular tissue comprises aligning the heart-tissue-conforming element with a heart valve commissure or a heart valve leaflet. Utilizing active alignment features may comprise pulsing balloons on the external surface of the heart-tissue-conforming element, e.g., winged balloons, in a manner that encourages the winged balloons to seat themselves in valve commissures, thereby aligning the heart-tissue-conforming element with respect to the heart valve.

In some cases, a method according to the present invention is a method of preparing the cardiovascular tissue for a heart valve replacement procedure or for placement of a prosthetic heart valve. That is, preparing tissue for a replacement heart valve may comprise disrupting calcium deposits in cardiovascular tissue or influencing tissue compliance, prior to implanting a heart valve. In other cases, the method is a method of delivering an active agent to the cardiovascular tissue or surrounding tissue. For example, an active agent may be present on an external surface of the heart-tissue-conforming element such that pulsing balloons of the heart-tissue-conforming element causes the active agent to dissolve into the cardiovascular tissue.

In some instances, methods of imparting pulsatile energy to cardiovascular tissue further comprise using an imaging technique to align the catheter assembly, e.g., the heart-tissue-conforming element, with cardiovascular tissue, e.g., a heart valve or diseased cardiovascular tissue or another a treatment site. Any convenient imaging technique capable of visualizing aspects of a catheter present in a lumen, such as a system according to the invention, may be applied. Imaging techniques may comprise, for example, ultrasound imaging, such as intravascular ultrasound technique, a light-based imaging technique, an angioplasty-based imaging technique or an optical coherence tomography-based technique or the like. In some cases, diagnostic X-ray imaging may be applied.

As described above, methods for imparting pulsatile energy to cardiovascular tissue of the invention may comprise a method for treating cardiovascular disease. In some cases, the method is a method for treating arteriosclerosis, meaning the thickening and stiffening of arterial walls. In some cases, methods of imparting pulsatile energy to cardiovascular tissue of the invention include a method for imparting pulsatile energy to cardiovascular tissue of a subject. The methods may be used for imparting pulsatile energy to cardiovascular tissue of any number of different subjects. In some instances, the subjects are “mammals” or “mammalian,” where these terms are used broadly to describe organisms which are within the class mammalia, including the orders carnivore (e.g., dogs and cats), rodentia (e.g., mice, guinea pigs and rats), and primates (e.g., humans, chimpanzees and monkeys). In some instances, the subjects are humans. For example, in some cases, a method of the present invention is a method of treating arteriosclerosis of a subject that is human.

FIG. 19 shows a graphical user interface (GUI) that an operator of a system of the present invention may interface with during a treatment procedure comprising imparting pulsatile energy to a heart valve when performing an embodiment of a method according to the invention. In this embodiment, the GUI may have several information zones such as a Fluoroscopy zone, Balloon Info zone, a Treatment Info zone and a Compliance zone. The Fluoroscopy zone presents imaging results of the heart-tissue-conforming element and the relevant cardiovascular tissue, i.e., the heart valve. Such imaging shows the interaction, i.e., engagement, between the heart-tissue-conforming element and the heart valve, e.g., whether the heart-tissue-conforming element is aligned as desired with the heart valve, as well as whether and to what extent a balloon of the heart-tissue-conforming element is inflated and how the corresponding cardiovascular tissue is responding. Balloon Info zone indicates information about the heart-tissue-conforming element or one or more balloons thereof, attached to the catheter. Such information includes, for example, a device type and size referring to the heart-tissue-conforming element or components thereof. Additional information that may be displayed includes heart-tissue-conforming element (or balloon thereof) diameter, length, nominal pressure, rated pressure as well as other characteristics, such as drug-coated or stent covered balloons or other aspects of a heart-tissue-conforming element. Additionally, heart-tissue-conforming element catheter connectivity information (i.e., whether a heart-tissue-conforming element has been connected or not) can be included in this section. Treatment Info zone indicates the important procedural characteristics that the operator employs during the procedure. Such characteristics include heart valve diameter and area as well as a valvular pressure gradient, i.e., a difference in pressure on either side of a heart valve. Other characteristics may be related to measurements indicating the degree of blood that is able to perfuse past the heart-tissue-conforming element to distal vessels. Some or all of this information may be updated or changed by the system and/or by the user, as the case may be. In the Compliance zone, various procedural plots may be displayed including a compliance plot and treatment plot. The compliance plot may include a nominal pressure-volume curve that may be provided with the heart-tissue-conforming element (or a balloon thereof). Additionally, a pressure-volume curve measured in-situ during the procedure may be plotted and updated throughout the procedure. The operator may use this plot to determine the effectiveness of treatment as a measure of compliance or efficacy. The pressure plot may include a display of pressure versus time. Other information that may be included (not shown) on the GUI include treatment status and intensity, ON/OFF switches, indicator LEDs, and the like.

### Kits

Also provided are kits that include systems, or one or more components thereof, e.g., as described above. As such, kits may include, in some instances, one or more of, a catheter assembly, a manifold assembly, a console assembly, with or without a potential energy source, e.g., a pressure source, or components thereof. The kit components may be present in packaging, which packaging may be sterile, as desired. Components of the kit may be disposable or reusable, as desired. In some cases, kits may comprise a plurality of components including multiple versions of the same component in different sizes, such as, for example, multiple catheters of varying diameters or heart-tissue-conforming elements configured for different aspects of cardiovascular tissue, e.g., for a heart valve.

Also present in the kit may be instructions for using the kit components. The instructions may be recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g., portable flash drive, DVD- or CD-ROM, etc. The instructions may take any form, including complete instructions for how to use the device or as a website address with which instructions posted on the world wide web may be accessed.

The following example(s) is/are offered by way of illustration and not by way of limitation.

### EXAMPLES

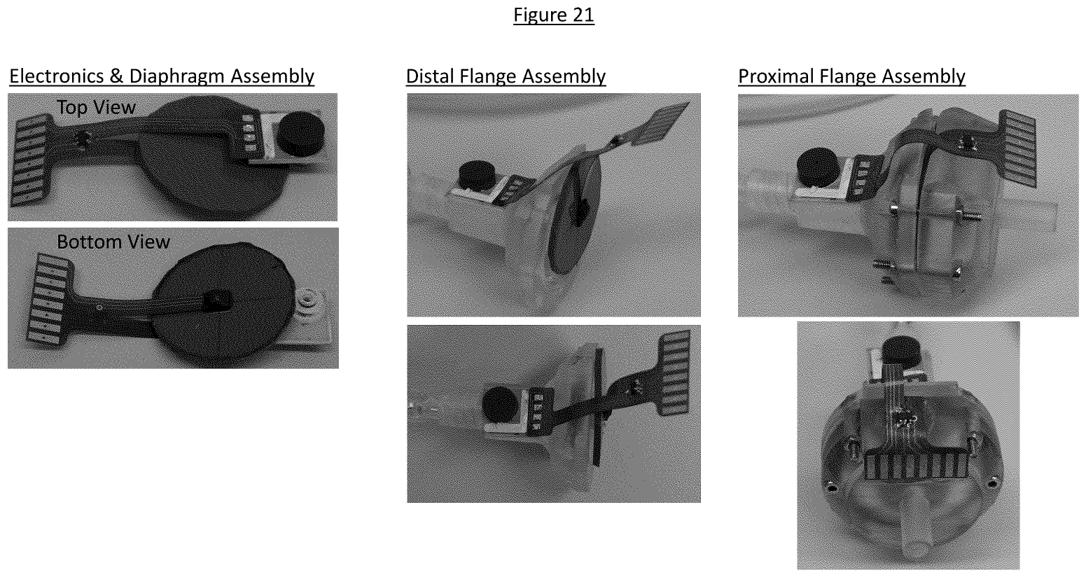

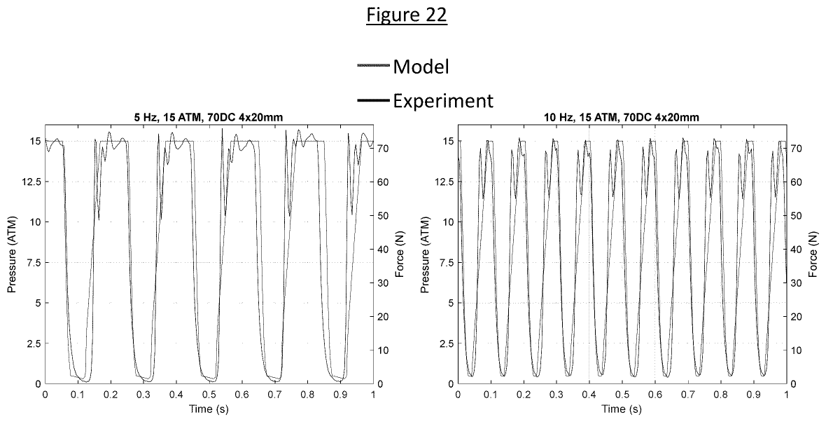

FIG. 20 provides a picture of aspects of a catheter assembly of a system according to an embodiment of the invention. FIG. 21 shows the assembly process for the connector of the catheter assembly shown in FIG. 20. The first step of the assembly process is to fix the electronic flexible printed circuit board assembly to the diaphragm and pressure sensor. For example, epoxy and solder, respectively, may be used. The pressure sensor and diaphragm may be fixed to the distal flange. Using an appropriate fixation technique (e.g., fasteners, welding, etc.), the proximal flange may be fixed to the distal flange. The electronic connector may be fixed to the front face of the proximal flange with epoxy, for example, so as to provide a reliable connection to the hand-held actuator. FIG. 22 represents testing performed on a single balloon of the described embodiment and physical assembly. The pressure is shown to increase while the force at the balloon increases as well. The force produced during oscillation matches the force produced during static inflation, indicating minimal attenuation by the system during pulsation.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112(6) is not invoked.

1. A system for imparting pulsatile energy to cardiovascular tissue, the system comprising:
(a) a console assembly comprising a potential source;
(b) a manifold assembly operably connected to an output of the console assembly, wherein the manifold assembly comprises an oscillator configured to generate pulse energy from energy transmitted from the potential source; and
(c) a catheter assembly operably connected to an output of the manifold assembly, wherein the catheter assembly comprises:
(i) a connector operably connecting the catheter assembly to the manifold assembly and configured to transduce a first pulse energy generated by the manifold assembly to a second pulse energy;
(ii) a catheter comprising a fluidic passage operably connected to the output of the connector and configured to transmit the second pulse energy; and
(iii) a heart-tissue-conforming element configured to receive the second pulse energy transmitted through the fluidic passage of the catheter to apply pulsatile energy to cardiovascular tissue.

2. The system according to claim 1, wherein the heart-tissue-conforming element is configured to engage heart valve tissue.

3-15. (canceled)

16. The system according to claim 1, wherein the heart-tissue-conforming element is located at a distal region of the catheter.

17. The system according to claim 1, wherein the heart-tissue-conforming element comprises a plurality of distal balloons arranged circumferentially around a rigid distal region of the catheter.

18. The system according to claim 17, wherein the distal balloons are configured to independently receive pulse energy generated by the manifold assembly.

19. The system according to claim 18, wherein
the fluidic passage of the catheter is a first fluidic passage, and
the catheter assembly comprises a plurality of fluidic passages, wherein each fluidic passage is operably connected to a corresponding distal balloon.

20. The system according to claim 19, wherein the plurality of fluidic passages comprises fluidic passages internal and external to the catheter.

21. The system according to claim 19, wherein
the connector is a first connector, and
the catheter assembly comprises a plurality of connectors, wherein each connector is operably connected to a corresponding fluidic passage of the catheter.

22. The system according to claim 21, wherein outputs of the connectors are connected to the catheter by a transition hub.

23. The system according to claim 22, wherein the transition hub couples outputs of the connectors to the fluidic passage of the catheter.

24. The system according to claim 22, wherein the transition hub couples outputs of the connectors to corresponding fluidic passages.

25. The system according to claim 17, wherein in an inflated state the distal balloons are configured to provide structural rigidity.

26-31. (canceled)

32. The system according to claim 17, wherein the distal balloons are coated with an active agent.

33. The system according to claim 17, wherein in an inflated state the distal balloons are arranged to leave space for fluid to pass between the distal balloons and the catheter.

34. The system according to claim 17, further comprising a membrane present at the rigid distal region of the catheter configured to cover the distal balloons.

35. The system according to claim 34, wherein the membrane is coated with an active agent.

36. The system according to claim 17, further comprising a plurality of lobes present on the distal balloons and extending radially beyond the distal balloons.

37-44. (canceled)

45. The system according to claim 1, wherein the catheter comprises a guidewire channel.

46. The system according to claim 1, wherein the catheter comprises a pressure sensor.

47. The system according to claim 46, wherein the pressure sensor is located at a distal region of the catheter.

48-235. (canceled)
